SEQUENCE LISTING (45 sequences)

SEQ ID NO: 1 - DNA, 99 bp, Homo sapiens
  misc_feature 1..99: note = human RHO promoter fragment
  source 1..99: mol_type = unassigned DNA
agaagccaattaggccctcagtttctgcagcggggattaatatgattatgaacacccccaatctcccagatgctgattcagccaggagcttaggagggg

SEQ ID NO: 2 - DNA, 350 bp, Homo sapiens
  misc_feature 1..350: note = human RHO promoter fragment
  source 1..350: mol_type = unassigned DNA
aaaccagaaagtctctagctgtccagaggacatagcacagaggcccatggtccctatttcaaacccaggccaccagactgagctgggaccttgggacagacaagtcatgcagaagttaggggaccttctcctcccttttcctggatcctgagtacctctcctccctgacctcaggcttcctcctagtgtcaccttggcccctcttagaagccaattaggccctcagtttctgcagcggggattaatatgattatgaacacccccaatctcccagatgctgattcagccaggagcttaggagggggaggtcactttataagggtctgggggggtcagaacccagagtcatc

SEQ ID NO: 3 - protein, 1245 aa, Homo sapiens
  REGION 1..1245: note = MISC_FEATURE - human CNGB1 protein
MLGWVQRVLPQPPGTPRKTKMQEEEEVEPEPEMEAEVEPEPNPEEAETESESMPPEESFKEEEVAVADPSPQETKEAALTSTISLRAQGAEISEMNSPSHRVLTWLMKGVEKVIPQPVHSITEDPAQILGHGSTGDTGCTDEPNEALEAQDTRPGLRLLLWLEQNLERVLPQPPKSSEVWRDEPAVATAPPGRPQEMGPKLQARETPSLPTPIPLQPKEEPKEAPAPEPQPGSQAQTSSLPPTRDPARLVAWVLHRLEMALPQPVLHGKIGEQEPDSPGICDVQTISILPGGQVEPDLVLEEVEPPWEDAHQDVSTSPQGTEVVPAYEEENKAVEKMPRELSRIEEEKEDEEEEEEEEEEEEEEEVTEVLLDSCVVSQVGVGQSEEDGTRPQSTSDQKLWEEVGEEAKKEAEEKAKEEAEEVAEEEAEKEPQDWAETKEEPEAEAEAASSGVPATKQHPEVQVEDTDADSCPLMAEENPPSTVLPPPSPAKSDTLIVPSSASGTHRKKLPSEDDEAEELKALSPAESPVVAWSDPTTPKDTDGQDRAASTASTNSAIINDRLQELVKLFKERTEKVKEKLIDPDVTSDEESPKPSPAKKAPEPAPDTKPAEAEPVEEEHYCDMLCCKFKHRPWKKYQFPQSIDPLTNLMYVLWLFFVVMAWNWNCWLIPVRWAFPYQTPDNIHHWLLMDYLCDLIYFLDITVFQTRLQFVRGGDIITDKKDMRNNYLKSRRFKMDLLSLLPLDFLYLKVGVNPLLRLPRCLKYMAFFEFNSRLESILSKAYVYRVIRTTAYLLYSLHLNSCLYYWASAYQGLGSTHWVYDGVGNSYIRCYYFAVKTLITIGGLPDPKTLFEIVFQLLNYFTGVFAFSVMIGQMRDVVGAATAGQTYYRSCMDSTVKYMNFYKIPKSVQNRVKTWYEYTWHSQGMLDESELMVQLPDKMRLDLAIDVNYNIVSKVALFQGCDRQMIFDMLKRLRSVVYLPNDYVCKKGEIGREMYIIQAGQVQVLGGPDGKSVLVTLKAGSVFGEISLLAVGGGNRRTANVVAHGFTNLFILDKKDLNEILVHYPESQKLLRKKARRMLRSNNKPKEEKSVLILPPRAGTPKLFNAALAMTGKMGGKGAKGGKLAHLRARLKELAALEAAAKQQELVEQAKSSQDVKGEEGSAAPDQHTHPKEAATDPPAPRTPPEPPGSPPSSPPPASLGRPEGEEEGPAEPEEHSVRICMSPGPEPGEQILSVKMPEEREEKAE

SEQ ID NO: 4 - DNA, 227 bp, synthetic construct
  misc_feature 1..227: note = SV40 polyadenylation signal
  source 1..227: mol_type = other DNA
ggccgcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 5 - DNA, 130 bp, synthetic construct
  misc_feature 1..130: note = L-ITR
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 6 - DNA, 131 bp, synthetic construct
  misc_feature 1..131: note = R-ITR
  source 1..131: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagc

SEQ ID NO: 7 - DNA, 10243 bp, synthetic construct
  misc_feature 1..10243: note = pGL2.0-hRHO194-hCNGB1a-SV40 vector molecule
  source 1..10243: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagcctctcctccctgacctcaggcttcctcctagtgtcaccttggcccctcttagaagccaattaggccctcagtttctgcagcggggattaatatgattatgaacacccccaatctcccagatgctgattcagccaggagcttaggagggggaggtcactttataagggtctgggggggtcagaacccagagtcatcactagtaacggccgccagtgtgctggaattcgcccttctccaccgccatgttgggctgggtccagagggtgctgcctcagcccccagggacccctcggaagaccaagatgcaggaggaagaggaagtggaaccagagccagagatggaggcggaggtggaaccagaaccgaatcctgaggaggccgagacagagtccgagtccatgccccccgaagagtcattcaaggaggaggaagtggctgtggcagacccaagccctcaggagaccaaggaggctgcccttacttccaccatatccctccgggcccagggcgctgagatttctgaaatgaatagtcccagccacagggtactgacctggctcatgaagggtgtagagaaggtgatcccgcagcctgttcacagcatcacggaggacccggctcagatcctggggcatggcagcactggggacacagggtgcacagatgaacccaatgaggcccttgaggcccaagacactaggcctgggctgcggctgcttctgtggctggagcagaatctggaaagagtgcttcctcagccccccaaatcctctgaggtctggagagatgagcctgcagttgctacagcgcctccaggacgcccccaggaaatggggcccaagctgcaggcccgggagaccccctccctgcccacacccatccccctgcagcccaaggaggaacccaaggaggcaccagctccagagccccagcccggctcccaggcccagacctcctccctgccaccaaccagggaccctgccaggctggtggcatgggtcctgcacaggctggagatggccttgccgcagccagtgctacatgggaaaataggggaacaggagcctgactcccctgggatatgtgatgtgcagaccatcagcatccttcctggaggacaagtggagcctgaccttgtcctagaggaggttgaaccgccctgggaggatgcccaccaggatgtcagtaccagcccacagggtacagaggtggttccagcttatgaagaagagaacaaagctgtggagaagatgcccagagagctgtcccggattgaagaggagaaagaagatgaggaggaggaagaggaagaggaggaggaggaggaagaggaggaggtgactgaggtgctgctggatagctgtgtggtgtcgcaggtgggcgtgggccagagtgaagaagacgggacccggccccagagcacttcagatcagaagctgtgggaggaagttggggaggaggccaagaaggaggctgaagagaaggccaaggaggaggccgaggaggtggctgaagaggaggctgaaaaggagccccaggactgggcggagaccaaggaggagcctgaggctgaggccgaggctgccagttcaggagtgcctgccacgaaacagcacccagaagtgcaggtggaagatactgatgctgatagctgccccctcatggcagaagagaatccaccctcaaccgtgttgccgccaccatctcctgccaaatcagacacccttatagtcccaagctcagcctcggggacacacaggaagaagctgccctctgaggatgatgaggctgaagagctcaaggcgttgtcaccagcagagtccccagtggttgcctggtctgaccccaccaccccgaaggacactgatggccaggaccgtgcggcctccacggccagcacaaatagcgccatcatcaacgaccggctccaggagctggtgaagctcttcaaggagcggacagagaaagtgaaggagaaactcattgaccctgacgtcacctctgatgaggagagccccaagccctccccagccaagaaagccccagagccagctccagacacaaagcccgctgaagccgagccagtggaagaggagcactattgcgacatgctctgctgcaagttcaaacaccgcccctggaagaagtaccagtttccccagagcattgacccgctgaccaacctgatgtatgtcctatggctgttcttcgtggtgatggcctggaattggaactgttggctgattcccgtgcgctgggccttcccctaccagaccccggacaacatccaccactggctgctgatggattacctatgcgacctcatctacttcctggacatcaccgtgttccagacacgcctgcagtttgtcagaggcggggacatcattacggacaaaaaggacatgcgaaataactacctgaagtctcgccgcttcaagatggacctgctcagcctcctgcccttggattttctctatttgaaagtcggtgtgaaccccctcctccgcctgccccgctgtttaaagtacatggccttcttcgagtttaacagccgcctggaatccatcctcagcaaagcctacgtgtacagggtcatcaggaccacagcctaccttctctacagcctgcatttgaattcctgtctttattactgggcatcggcctatcagggcctcggctccactcactgggtttacgatggcgtgggaaacagttatattcgctgttactactttgctgtgaagaccctcatcaccatcggggggctgcctgaccccaagacactctttgaaattgtcttccagctgctgaattatttcacgggcgtctttgctttctctgtgatgatcggacagatgagagatgtggtaggggccgccaccgcgggacagacctactaccgcagctgcatggacagcacggtgaagtacatgaatttctacaagatccccaagtccgtgcagaaccgcgtcaagacctggtacgagtacacctggcactcgcaaggcatgctggatgagtcagagctgatggtgcagcttccagacaagatgcggctggacctcgccatcgacgtgaactacaacatcgttagcaaagtcgcactctttcagggctgtgaccggcagatgatctttgacatgctgaagaggcttcgctctgttgtctacctgcccaacgactatgtgtgcaagaagggggagatcggccgtgagatgtacatcatccaggcagggcaagtgcaggtcttgggcggccctgatgggaaatctgtgctggtgacgctgaaagctggatctgtgtttggagaaataagcttgctggctgttgggggcgggaaccggcgcacggccaacgtggtggcgcacgggtttaccaacctcttcatcctggataagaaggacctgaatgagattttggtgcattatcctgagtctcagaagttactccggaagaaagccaggcgcatgctgagaagcaacaataagcccaaggaggagaagagcgtgctgatccttccaccccgggcgggcaccccaaagctcttcaacgctgccctcgctatgacaggaaagatgggtggcaagggggcaaaaggcggcaaacttgctcacctccgggcccggctcaaagaactggccgcgctggaggcggctgcaaagcagcaagagttggtggaacaggccaagagctcgcaagacgtcaagggagaggaaggctccgccgccccagaccagcacacgcacccaaaggaggccgccaccgacccacccgcgccccggacgccccccgagcccccggggtctccaccgagctctccaccgcctgcctcccttgggaggccggagggagaggaggaggggccggccgagcccgaagagcactcggtgaggatctgcatgagcccgggcccggagccgggagagcagatcctgtcggtgaagatgccggaggaaagggaggagaaggcggagtaaggtggggtgaggcggatccatggccgcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtcgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgactgtggaatgtgtgtcagttaggcgacataggtgatctatgtagaagcctagtggaacaggttagtttgagtagctttagaatgtaaattctgggatcatagtgtagtaatctctaattaacggtgacggtttgtaagacaggtcttcgcaaaatcaagcggcaggtgatttcaacagattcttgctgatggtttaggcgtacaatgccctgaagaataagtaagagaatagcactcctcgtcgcctagaattacctaccggcgtccaccataccttcgattatcgcgcccactctcccattagtcggcacaggtggatgtgttgcgatagcccgctaagatattctaaggcgtaacgcagatgaatattctacagagttgccataggcgttgaacgcttcacggacgataggaatgttgcgtatagagcgtgagtcatcgaagtggtgtatacactcgtacttaacatctagcccggctctatcagtacaccagtgccttgaatgacatactcatcattaaactttctcaacagtcaaacgaccaagtgcatttccaaggagtgcgaaggagattcattctctcgccagcactgtaataggcactaaaagagtgaagataagctagagtgccgtgctaagacggtgtcggaacaaagcggtcttacggtcagtcgtatttcctgtcgagtcccgtccagttgagcgtatcactcccagtgtactagcaagccgagaaggctgtgcttggagtcaatcggatgtaggatggtctccagacaccgggccaccactcttcacgcctagaagcatagaacgtcgagcagacatcaaagtcttagtaccggacgtgccgtttcactgcgaatattacctgaagctgtaccgttattgcggagcaaagtgacagtgctgctcttatcatatttgtattgacgacagccgccttcgcggtttcctcagactctagatcgaatacaggcttattgtaggcagaggcacgcccttgttagtggctgcggcaatatcttccgatccccttgtctaaccatgaatcaattctctcatttgaagaccctaatatgtcatcattagtgtttcaaatgccaccaaataccgcctagaaatgtctatgatgtgtgtccactagaagttgattcacaaacgactgctagaatcgcgtgatagggcatcttgaagtttacattgttgtatcgcaaggtactccgatcttaatggatgcgaagtggtacggatgcaatcaagcgcgtgagagcggtacattagagcgttcacctacgctacgctaacgggcgattctgataagaatgcacattgcgtcgattcataagatgtctcgaccgcatgcgcaacttgtgaagtgtctactatccctaagcgcatatctcgcacagtaaccgaatatgtcggcatctgatgttaccgttgagttagtgttcagctcacggaacttattgtatgagtagagatttgtaagagctgttagttagctcgctcagctaatagttgcccacacaacgtcaaattagagaacggtcgtaacattatcggtggttctctaactactatcagtacccacgactcgactctgccgcagctaggtatcgcctgaaagccagtcagcgttaaggagtgctctgaccaggacaacaggcgtagtgagagttacttgttcgttgctcttccgactcggacctgagttcgccaacgacccacttgaggtctgagccggtgaagagaagtaagcatctcgttcgcagcttgccagcactttcagaacatgacccctatttgtttatttttctaaatacattcaaatatgtatccgctcatgagacaataaccctgataaatgcttcaataatattgaaaaaggaagagtggccgcctcggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggtaccatgatgcgtgcatggtagaatgactcttgataacggacttcgactaggcaatatcccttgtcaacttgtcgaggagaaaagtattgactgaagcgctcccggcacaacggccaaagaagtctcagcaatgttcttatttccgaatgacatgcgtctccttgcgggtaaatcgccgaccgcaaaacttaggagccaggatacagataggtctaacttaggttaagggagtaaatcctgggatcgttcagttgtaaccatatacttacgctggggcttctccggcggatgttactgtcaccaaccacgagatttgaagtaaacgcatgattgagcacatagccgcgctatccgacaatctccaaattgataacataccgttccatgaaggccagaattacttaccggccctttccatgcgtgcgccataccgcactctgcgcttatccgtccgaggggagagtgtgcgatcctccgttaagatattctcacgtatgacgtagctatgtattgtgcagaggtagcgaaggcgttgaacacttcacagatggtggggattcgggcaaagggcgtgataacttggggactaacataggcgtaaactacgatggcaccaactcaatcgcagctcgtgcgccctgaatcaacgtactcatctcaactgattctcggcaatctacggagcgacttgattatcaacacctgtctagcagttctaatcttctgccaacatcgtacatagcctccaagagattatcatacctatcggcacagaagtgacacgacgccgaagggtagcggacttctggtcaaccacaattccccaggggacaggtcctgcggtgcgcatcactttgtaagtgcaagcaacccaagtgagcccagcctggactgagctggttcctgtgtcaggtcgaggctggggatgacagctcttgtaaacatagtgatcaagcgtggcgtcgaacggtcgagaaactcatagtacctcgggtagcaacttactcaggttattgcttgaagctgtactatttcaggagcgctgaaggtctcttcttctgtagactgaactcgcaagggtcgtgaagtcggttccttcaatgcttaacaagaacaaaggcttactgtgcagactggaacgcccatctagcggctcgcgtcttgaatgctcggtcccctttgtcattgcggatacaatccatttccctcattcaccagcttgcgaagtctacattgagtagacgaatgcgacctagaagaggtgcgcttcagaacttgtgaggagtggttgatgctctatactccatttggtgtttcgtgcatcaccgcgataggctgacaagaggtcttgaacattgaatagcaaggcacttccggtctcatagaagagagcacgggataaggtacgcgcgtggtacgggaggatcaaggggctacacgatagaaaggcttctccctcactcgctaggaggcaaatgcagaacgctggttactactacgatacgtgaaacttgtccaacggttgcccaaagtgttaagtgtctatcaccctagtgccgtttcccggagaaaacgccaggttgaatccgcatttgaagctacgatggtgaagtctgggtcgagcgcgccgcatgttgattgcgtgagtaggctcgaccaagaaccgctagtagcgtcgctgtagaaatagttctcgacagaccgtcgagtttagaaaatggtagcagcattgttcgcatctcaatcaagtatggattacggtgtttacactgtcctgcggctacccatcgcctgaaatccagctcgtgtcaagccattgcctctccgggacgccgcatgaagtaactacatataccttgcacgggttgactgcggtccgttcagactcgaccaaggacacaatccagcgatcggtgcgggcctcttcgctattacgc

SEQ ID NO: 8 - DNA, 3738 bp, Homo sapiens
  misc_feature 1..3738: note = human CNGB1 gerne
  source 1..3738: mol_type = unassigned DNA
atgttgggctgggtccagagggtgctgcctcagcccccagggacccctcggaagaccaagatgcaggaggaagaggaagtggaaccagagccagagatggaggcggaggtggaaccagaaccgaatcctgaggaggccgagacagagtccgagtccatgccccccgaagagtcattcaaggaggaggaagtggctgtggcagacccaagccctcaggagaccaaggaggctgcccttacttccaccatatccctccgggcccagggcgctgagatttctgaaatgaatagtcccagccacagggtactgacctggctcatgaagggtgtagagaaggtgatcccgcagcctgttcacagcatcacggaggacccggctcagatcctggggcatggcagcactggggacacagggtgcacagatgaacccaatgaggcccttgaggcccaagacactaggcctgggctgcggctgcttctgtggctggagcagaatctggaaagagtgcttcctcagccccccaaatcctctgaggtctggagagatgagcctgcagttgctacagcgcctccaggacgcccccaggaaatggggcccaagctgcaggcccgggagaccccctccctgcccacacccatccccctgcagcccaaggaggaacccaaggaggcaccagctccagagccccagcccggctcccaggcccagacctcctccctgccaccaaccagggaccctgccaggctggtggcatgggtcctgcacaggctggagatggccttgccgcagccagtgctacatgggaaaataggggaacaggagcctgactcccctgggatatgtgatgtgcagaccatcagcatccttcctggaggacaagtggagcctgaccttgtcctagaggaggttgaaccgccctgggaggatgcccaccaggatgtcagtaccagcccacagggtacagaggtggttccagcttatgaagaagagaacaaagctgtggagaagatgcccagagagctgtcccggattgaagaggagaaagaagatgaggaggaggaagaggaagaggaggaggaggaggaagaggaggaggtgactgaggtgctgctggatagctgtgtggtgtcgcaggtgggcgtgggccagagtgaagaagacgggacccggccccagagcacttcagatcagaagctgtgggaggaagttggggaggaggccaagaaggaggctgaagagaaggccaaggaggaggccgaggaggtggctgaagaggaggctgaaaaggagccccaggactgggcggagaccaaggaggagcctgaggctgaggccgaggctgccagttcaggagtgcctgccacgaaacagcacccagaagtgcaggtggaagatactgatgctgatagctgccccctcatggcagaagagaatccaccctcaaccgtgttgccgccaccatctcctgccaaatcagacacccttatagtcccaagctcagcctcggggacacacaggaagaagctgccctctgaggatgatgaggctgaagagctcaaggcgttgtcaccagcagagtccccagtggttgcctggtctgaccccaccaccccgaaggacactgatggccaggaccgtgcggcctccacggccagcacaaatagcgccatcatcaacgaccggctccaggagctggtgaagctcttcaaggagcggacagagaaagtgaaggagaaactcattgaccctgacgtcacctctgatgaggagagccccaagccctccccagccaagaaagccccagagccagctccagacacaaagcccgctgaagccgagccagtggaagaggagcactattgcgacatgctctgctgcaagttcaaacaccgcccctggaagaagtaccagtttccccagagcattgacccgctgaccaacctgatgtatgtcctatggctgttcttcgtggtgatggcctggaattggaactgttggctgattcccgtgcgctgggccttcccctaccagaccccggacaacatccaccactggctgctgatggattacctatgcgacctcatctacttcctggacatcaccgtgttccagacacgcctgcagtttgtcagaggcggggacatcattacggacaaaaaggacatgcgaaataactacctgaagtctcgccgcttcaagatggacctgctcagcctcctgcccttggattttctctatttgaaagtcggtgtgaaccccctcctccgcctgccccgctgtttaaagtacatggccttcttcgagtttaacagccgcctggaatccatcctcagcaaagcctacgtgtacagggtcatcaggaccacagcctaccttctctacagcctgcatttgaattcctgtctttattactgggcatcggcctatcagggcctcggctccactcactgggtttacgatggcgtgggaaacagttatattcgctgttactactttgctgtgaagaccctcatcaccatcggggggctgcctgaccccaagacactctttgaaattgtcttccagctgctgaattatttcacgggcgtctttgctttctctgtgatgatcggacagatgagagatgtggtaggggccgccaccgcgggacagacctactaccgcagctgcatggacagcacggtgaagtacatgaatttctacaagatccccaagtccgtgcagaaccgcgtcaagacctggtacgagtacacctggcactcgcaaggcatgctggatgagtcagagctgatggtgcagcttccagacaagatgcggctggacctcgccatcgacgtgaactacaacatcgttagcaaagtcgcactctttcagggctgtgaccggcagatgatctttgacatgctgaagaggcttcgctctgttgtctacctgcccaacgactatgtgtgcaagaagggggagatcggccgtgagatgtacatcatccaggcagggcaagtgcaggtcttgggcggccctgatgggaaatctgtgctggtgacgctgaaagctggatctgtgtttggagaaataagcttgctggctgttgggggcgggaaccggcgcacggccaacgtggtggcgcacgggtttaccaacctcttcatcctggataagaaggacctgaatgagattttggtgcattatcctgagtctcagaagttactccggaagaaagccaggcgcatgctgagaagcaacaataagcccaaggaggagaagagcgtgctgatccttccaccccgggcgggcaccccaaagctcttcaacgctgccctcgctatgacaggaaagatgggtggcaagggggcaaaaggcggcaaacttgctcacctccgggcccggctcaaagaactggccgcgctggaggcggctgcaaagcagcaagagttggtggaacaggccaagagctcgcaagacgtcaagggagaggaaggctccgccgccccagaccagcacacgcacccaaaggaggccgccaccgacccacccgcgccccggacgccccccgagcccccggggtctccaccgagctctccaccgcctgcctcccttgggaggccggagggagaggaggaggggccggccgagcccgaagagcactcggtgaggatctgcatgagcccgggcccggagccgggagagcagatcctgtcggtgaagatgccggaggaaagggaggagaaggcggagtaa

SEQ ID NO: 9 - DNA, 194 bp, Homo sapiens
  misc_feature 1..194: note = human RHO promoter fragment
  source 1..194: mol_type = unassigned DNA
tctcctccctgacctcaggcttcctcctagtgtcaccttggcccctcttagaagccaattaggccctcagtttctgcagcggggattaatatgattatgaacacccccaatctcccagatgctgattcagccaggagcttaggagggggaggtcactttataagggtctgggggggtcagaacccagagtcatc

SEQ ID NO: 10 - protein, 1065 aa, Homo sapiens [sequence omitted]

SEQ ID NO: 11 - protein, 384 aa, Homo sapiens
  REGION 1..384: note = MISC_FEATURE - AIPL1 (Aryl-hydrocarbon-interacting protein-like 1)
MDAALLLNVEGVKKTILHGGTGELPNFITGSRVIFHFRTMKCDEERTVIDDSRQVGQPMHIIIGNMFKLEVWEILLTSMRVHEVAEFWCDTIHTGVYPILSRSLRQMAQGKDPTEWHVHTCGLANMFAYHTLGYEDLDELQKEPQPLVFVIELLQVDAPSDYQRETWNLSNHEKMKAVPVLHGEGNRLFKLGRYEEASSKYQEAIICLRNLQTKEKPWEVQWLKLEKMINTLILNYCQCLLKKEEYYEVLEHTSDILRHHPGIVKAYYVRARAHAEVWNEAEAKADLQKVLELEPSMQKAVRRELRLLENRMAEKQEEERLRCRNMLSQGATQPPAEPPTEPPAQSSTEPPAEPPTAPSAELSAGPPAEPATEPPPSPGHSLQH

SEQ ID NO: 12 - protein, 585 aa, Homo sapiens
  REGION 1..585: note = MISC_FEATURE - BEST1 (Bestrophin-1), isoform 1
MTITYTSQVANARLGSFSRLLLCWRGSIYKLLYGEFLIFLLCYYIIRFIYRLALTEEQQLMFEKLTLYCDSYIQLIPISFVLGFYVTLVVTRWWNQYENLPWPDRLMSLVSGFVEGKDEQGRLLRRTLIRYANLGNVLILRSVSTAVYKRFPSAQHLVQAGFMTPAEHKQLEKLSLPHNMFWVPWVWFANLSMKAWLGGRIRDPILLQSLLNEMNTLRTQCGHLYAYDWISIPLVYTQVVTVAVYSFFLTCLVGRQFLNPAKAYPGHELDLVVPVFTFLQFFFYVGWLKVAEQLINPFGEDDDDFETNWIVDRNLQVSLLAVDEMHQDLPRMEPDMYWNKPEPQPPYTAASAQFRRASFMGSTFNISLNKEEMEFQPNQEDEEDAHAGIIGRFLGLQSHDHHPPRANSRTKLLWPKRESLLHEGLPKNHKAAKQNVRGQEDNKAWKLKAVDAFKSAPLYQRPGYYSAPQTPLSPTPMFFPLEPSAPSKLHSVTGIDTKDKSLKTVSSGAKKSFELLSESDGALMEHPEVSQVRRKTVEFNLTDMPEIPENHLKEPLEQSPTNIHTTLKDHMDPYWALENRDEAHS

SEQ ID NO: 13 - protein, 1977 aa, Homo sapiens
  REGION 1..1977: note = MISC_FEATURE - CACNA1F (Voltage-dependent L-type calcium channel subunit alpha-1F), isoform 1
MSESEGGKDTTPEPSPANGAGPGPEWGLCPGPPAVEGESSGASGLGTPKRRNQHSKHKTVAVASAQRSPRALFCLTLANPLRRSCISIVEWKPFDILILLTIFANCVALGVYIPFPEDDSNTANHNLEQVEYVFLVIFTVETVLKIVAYGLVLHPSAYIRNGWNLLDFIIVVVGLFSVLLEQGPGRPGDAPHTGGKPGGFDVKALRAFRVLRPLRLVSGVPSLHIVLNSIMKALVPLLHIALLVLFVIIIYAIIGLELFLGRMHKTCYFLGSDMEAEEDPSPCASSGSGRACTLNQTECRGRWPGPNGGITNFDNFFFAMLTVFQCVTMEGWTDVLYWMQDAMGYELPWVYFVSLVIFGSFFVLNLVLGVLSGEFSKEREKAKARGDFQKQREKQQMEEDLRGYLDWITQAEELDMEDPSADDNLGSMAEEGRAGHRPQLAELTNRRRGRLRWFSHSTRSTHSTSSHASLPASDTGSMTETQGDEDEEEGALASCTRCLNKIMKTRVCRRLRRANRVLRARCRRAVKSNACYWAVLLLVFLNTLTIASEHHGQPVWLTQIQEYANKVLLCLFTVEMLLKLYGLGPSAYVSSFFNRFDCFVVCGGILETTLVEVGAMQPLGISVLRCVRLLRIFKVTRHWASLSNLVASLLNSMKSIASLLLLLFLFIIIFSLLGMQLFGGKFNFDQTHTKRSTFDTFPQALLTVFQILTGEDWNVVMYDGIMAYGGPFFPGMLVCIYFIILFICGNYILLNVFLAIAVDNLASGDAGTAKDKGGEKSNEKDLPQENEGLVPGVEKEEEEGARREGADMEEEEEEEEEEEEEEEEEGAGGVELLQEVVPKEKVVPIPEGSAFFCLSQTNPLRKGCHTLIHHHVFTNLILVFIILSSVSLAAEDPIRAHSFRNHILGYFDYAFTSIFTVEILLKMTVFGAFLHRGSFCRSWFNMLDLLVVSVSLISFGIHSSAISVVKILRVLRVLRPLRAINRAKGLKHVVQCVFVAIRTIGNIMIVTTLLQFMFACIGVQLFKGKFYTCTDEAKHTPQECKGSFLVYPDGDVSRPLVRERLWVNSDFNFDNVLSAMMALFTVSTFEGWPALLYKAIDAYAEDHGPIYNYRVEISVFFIVYIIIIAFFMMNIFVGFVIITFRAQGEQEYQNCELDKNQRQCVEYALKAQPLRRYIPKNPHQYRVWATVNSAAFEYLMFLLILLNTVALAMQHYEQTAPFNYAMDILNMVFTGLFTIEMVLKIIAFKPKHYFTDAWNTFDALIVVGSIVDIAVTEVNNGGHLGESSEDSSRISITFFRLFRVMRLVKLLSKGEGIRTLLWTFIKSFQALPYVALLIAMIFFIYAVIGMQMFGKVALQDGTQINRNNNFQTFPQAVLLLFRCATGEAWQEIMLASLPGNRCDPESDFGPGEEFTCGSNFAIAYFISFFMLCAFLIINLFVAVIMDNFDYLTRDWSILGPHHLDEFKRIWSEYDPGAKGRIKHLDVVALLRRIQPPLGFGKLCPHRVACKRLVAMNMPLNSDGTVTFNATLFALVRTSLKIKTEGNLEQANQELRIVIKKIWKRMKQKLLDEVIPPPDEEEVTVGKFYATFLIQDYFRKFRRRKEKGLLGNDAAPSTSSALQAGLRSLQDLGPEMRQALTCDTEEEEEEGQEGVEEEDEKDLETNKATMVSQPSARRGSGISVSLPVGDRLPDSLSFGPSDDDRGTPTSSQPSVPQAGSNTHRRGSGALIFTIPEEGNSQPKGTKGQNKQDEDEEVPDRLSYLDEQAGTPPCSVLLPPHRAQRYMDGHLVPRRRLLPPTPAGRKPSFTIQCLQRQGSCEDLPIPGTYHRGRNSGPNRAQGSWATPPQRGRLLYAPLLLVEEGAAGEGYLGRSSGPLRTFTCLHVPGTHSDPSHGKRGSADSLVEAVLISEGLGLFARDPRFVALAKQEIADACRLTLDEMDNAASDLLAQGTSSLYSDEESILSRFDEEDLGDEMACVHAL

SEQ ID NO: 14 - protein, 438 aa, Homo sapiens
  REGION 1..438: note = MISC_FEATURE - CLN3 (Battenin), isoform 1
MGGCAGSRRRFSDSEGEETVPEPRLPLLDHQGAHWKNAVGFWLLGLCNNFSYVVMLSAAHDILSHKRTSGNQSHVDPGPTPIPHNSSSRFDCNSVSTAAVLLADILPTLVIKLLAPLGLHLLPYSPRVLVSGICAAGSFVLVAFSHSVGTSLCGVVFASISSGLGEVTFLSLTAFYPRAVISWWSSGTGGAGLLGALSYLGLTQAGLSPQQTLLSMLGIPALLLASYFLLLTSPEAQDPGGEEEAESAARQPLIRTEAPESKPGSSSSLSLRERWTVFKGLLWYIVPLVVVYFAEYFINQGLFELLFFWNTSLSHAQQYRWYQMLYQAGVFASRSSLRCCRIRFTWALALLQCLNLVFLLADVWFGFLPSIYLVFLIILYEGLLGGAAYVNTFHNIALETSDEHREFAMAATCISDTLGISLSGLLALPLHDFLCQLS

SEQ ID NO: 15 - protein, 232 aa, Homo sapiens
  REGION 1..232: note = MISC_FEATURE - CLRN1 (Clarin-1)
MPSQQKKIIFCMAGVFSFACALGVVTALGTPLWIKATVLCKTGALLVNASGQELDKFMGEMQYGLFHGEGVRQCGLGARPFRFSFFPDLLKAIPVSIHVNVILFSAILIVLTMVGTAFFMYNAFGKPFETLHGPLGLYLLSFISGSCGCLVMILFASEVKIHHLSEKIANYKEGTYVYKTQSEKYTTSFWVIFFCFFVHFLNGLLIRLAGFQFPFAKSKDAETTNVAADLMY

SEQ ID NO: 16 - protein, 690 aa, Homo sapiens
  REGION 1..690: note = MISC_FEATURE - CNGA1 (cGMP-gated cation channel alpha-1), isoform 1
MKLSMKNNIINTQQSFVTMPNVIVPDIEKEIRRMENGACSSFSEDDDSASTSEESENENPHARGSFSYKSLRKGGPSQREQYLPGAIALFNVNNSSNKDQEPEEKKKKKKEKKSKSDDKNENKNDPEKKKKKKDKEKKKKEEKSKDKKEEEKKEVVVIDPSGNTYYNWLFCITLPVMYNWTMVIARACFDELQSDYLEYWLILDYVSDIVYLIDMFVRTRTGYLEQGLLVKEELKLINKYKSNLQFKLDVLSLIPTDLLYFKLGWNYPEIRLNRLLRFSRMFEFFQRTETRTNYPNIFRISNLVMYIVIIIHWNACVFYSISKAIGFGNDTWVYPDINDPEFGRLARKYVYSLYWSTLTLTTIGETPPPVRDSEYVFVVVDFLIGVLIFATIVGNIGSMISNMNAARAEFQARIDAIKQYMHFRNVSKDMEKRVIKWFDYLWTNKKTVDEKEVLKYLPDKLRAEIAINVHLDTLKKVRIFADCEAGLLVELVLKLQPQVYSPGDYICKKGDIGREMYIIKEGKLAVVADDGVTQFVVLSDGSYFGEISILNIKGSKAGNRRTANIKSIGYSDLFCLSKDDLMEALTEYPDAKTMLEEKGKQILMKDGLLDLNIANAGSDPKDLEEKVTRMEGSVDLLQTRFARILAEYESMQQKLKQRLTKVEKFLKPLIDTEFSSIEGPGAESGPIDST

SEQ ID NO: 17 - protein, 2479 aa, Homo sapiens
  REGION 1..2479: note = MISC_FEATURE - CEP290 (centrosomal protein of 290 kDa), isoform 1
MPPNINWKEIMKVDPDDLPRQEELADNLLISLSKVEVNELKSEKQENVIHLFRITQSLMKMKAQEVELALEEVEKAGEEQAKFENQLKTKVMKLENELEMAQQSAGGRDTRFLRNEICQLEKQLEQKDRELEDMEKELEKEKKVNEQLALRNEEAENENSKLRRENKRLKKKNEQLCQDIIDYQKQIDSQKETLLSRRGEDSDYRSQLSKKNYELIQYLDEIQTLTEANEKIEVQNQEMRKNLEESVQEMEKMTDEYNRMKAIVHQTDNVIDQLKKENDHYQLQVQELTDLLKSKNEEDDPIMVAVNAKVEEWKLILSSKDDEIIEYQQMLHNLREKLKNAQLDADKSNVMALQQGIQERDSQIKMLTEQVEQYTKEMEKNTCIIEDLKNELQRNKGASTLSQQTHMKIQSTLDILKEKTKEAERTAELAEADAREKDKELVEALKRLKDYESGVYGLEDAVVEIKNCKNQIKIRDREIEILTKEINKLELKISDFLDENEALRERVGLEPKTMIDLTEFRNSKHLKQQQYRAENQILLKEIESLEEERLDLKKKIRQMAQERGKRSATSGLTTEDLNLTENISQGDRISERKLDLLSLKNMSEAQSKNEFLSRELIEKERDLERSRTVIAKFQNKLKELVEENKQLEEGMKEILQAIKEMQKDPDVKGGETSLIIPSLERLVNAIESKNAEGIFDASLHLKAQVDQLTGRNEELRQELRESRKEAINYSQQLAKANLKIDHLEKETSLLRQSEGSNVVFKGIDLPDGIAPSSASIINSQNEYLIHLLQELENKEKKLKNLEDSLEDYNRKFAVIRHQQSLLYKEYLSEKETWKTESKTIKEEKRKLEDQVQQDAIKVKEYNNLLNALQMDSDEMKKILAENSRKITVLQVNEKSLIRQYTTLVELERQLRKENEKQKNELLSMEAEVCEKIGCLQRFKEMAIFKIAALQKVVDNSVSLSELELANKQYNELTAKYRDILQKDNMLVQRTSNLEHLECENISLKEQVESINKELEITKEKLHTIEQAWEQETKLGNESSMDKAKKSITNSDIVSISKKITMLEMKELNERQRAEHCQKMYEHLRTSLKQMEERNFELETKFAELTKINLDAQKVEQMLRDELADSVSKAVSDADRQRILELEKNEMELKVEVSKLREISDIARRQVEILNAQQQSRDKEVESLRMQLLDYQAQSDEKSLIAKLHQHNVSLQLSEATALGKLESITSKLQKMEAYNLRLEQKLDEKEQALYYARLEGRNRAKHLRQTIQSLRRQFSGALPLAQQEKFSKTMIQLQNDKLKIMQEMKNSQQEHRNMENKTLEMELKLKGLEELISTLKDTKGAQKVINWHMKIEELRLQELKLNRELVKDKEEIKYLNNIISEYERTISSLEEEIVQQNKFHEERQMAWDQREVDLERQLDIFDRQQNEILNAAQKFEEATGSIPDPSLPLPNQLEIALRKIKENIRIILETRATCKSLEEKLKEKESALRLAEQNILSRDKVINELRLRLPATAEREKLIAELGRKEMEPKSHHTLKIAHQTIANMQARLNQKEEVLKKYQRLLEKAREEQREIVKKHEEDLHILHHRLELQADSSLNKFKQTAWDLMKQSPTPVPTNKHFIRLAEMEQTVAEQDDSLSSLLVKLKKVSQDLERQREITELKVKEFENIKLQLQENHEDEVKKVKAEVEDLKYLLDQSQKESQCLKSELQAQKEANSRAPTTTMRNLVERLKSQLALKEKQQKALSRALLELRAEMTAAAEERIISATSQKEAHLNVQQIVDRHTRELKTQVEDLNENLLKLKEALKTSKNRENSLTDNLNDLNNELQKKQKAYNKILREKEEIDQENDELKRQIKRLTSGLQGKPLTDNKQSLIEELQRKVKKLENQLEGKVEEVDLKPMKEKNAKEELIRWEEGKKWQAKIEGIRNKLKEKEGEVFTLTKQLNTLKDLFAKADKEKLTLQRKLKTTGMTVDQVLGIRALESEKELEELKKRNLDLENDILYMRAHQALPRDSVVEDLHLQNRYLQEKLHALEKQFSKDTYSKPSISGIESDDHCQREQELQKENLKLSSENIELKFQLEQANKDLPRLKNQVRDLKEMCEFLKKEKAEVQRKLGHVRGSGRSGKTIPELEKTIGLMKKVVEKVQRENEQLKKASGILTSEKMANIEQENEKLKAELEKLKAHLGHQLSMHYESKTKGTEKIIAENERLRKELKKETDAAEKLRIAKNNLEILNEKMTVQLEETGKRLQFAESRGPQLEGADSKSWKSIVVTRMYETKLKELETDIAKKNQSITDLKQLVKEATEREQKVNKYNEDLEQQIKILKHVPEGAETEQGLKRELQVLRLANHQLDKEKAELIHQIEANKDQSGAESTIPDADQLKEKIKDLETQLKMSDLEKQHLKEEIKKLKKELENFDPSFFEEIEDLKYNYKEEVKKNILLEEKVKKLSEQLGVELTSPVAASEEFEDEEESPVNFPIY

SEQ ID NO: 18 - protein, 1406 aa, Homo sapiens
  REGION 1..1406: note = MISC_FEATURE - CRB1 (protein crumbs homolog 1), isoform 1
MALKNINYLLIFYLSFSLLIYIKNSFCNKNNTRCLSNSCQNNSTCKDFSKDNDCSCSDTANNLDKDCDNMKDPCFSNPCQGSATCVNTPGERSFLCKCPPGYSGTICETTIGSCGKNSCQHGGICHQDPIYPVCICPAGYAGRFCEIDHDECASSPCQNGAVCQDGIDGYSCFCVPGYQGRHCDLEVDECASDPCKNEATCLNEIGRYTCICPHNYSGVNCELEIDECWSQPCLNGATCQDALGAYFCDCAPGFLGDHCELNTDECASQPCLHGGLCVDGENRYSCNCTGSGFTGTHCETLMPLCWSKPCHNNATCEDSVDNYTCHCWPGYTGAQCEIDLNECNSNPCQSNGECVELSSEKQYGRITGLPSSFSYHEASGYVCICQPGFTGIHCEEDVNECSSNPCQNGGTCENLPGNYTCHCPFDNLSRTFYGGRDCSDILLGCTHQQCLNNGTCIPHFQDGQHGFSCLCPSGYTGSLCEIATTLSFEGDGFLWVKSGSVTTKGSVCNIALRFQTVQPMALLLFRSNRDVFVKLELLSGYIHLSIQVNNQSKVLLFISHNTSDGEWHFVEVIFAEAVTLTLIDDSCKEKCIAKAPTPLESDQSICAFQNSFLGGLPVGMTSNGVALLNFYNMPSTPSFVGCLQDIKIDWNHITLENISSGSSLNVKAGCVRKDWCESQPCQSRGRCINLWLSYQCDCHRPYEGPNCLREYVAGRFGQDDSTGYVIFTLDESYGDTISLSMFVRTLQPSGLLLALENSTYQYIRVWLERGRLAMLTPNSPKLVVKFVLNDGNVHLISLKIKPYKIELYQSSQNLGFISASTWKIEKGDVIYIGGLPDKQETELNGGFFKGCIQDVRLNNQNLEFFPNPTNNASLNPVLVNVTQGCAGDNSCKSNPCHNGGVCHSRWDDFSCSCPALTSGKACEEVQWCGFSPCPHGAQCQPVLQGFECIANAVFNGQSGQILFRSNGNITRELTNITFGFRTRDANVIILHAEKEPEFLNISIQDSRLFFQLQSGNSFYMLSLTSLQSVNDGTWHEVTLSMTDPLSQTSRWQMEVDNETPFVTSTIATGSLNFLKDNTDIYVGDRAIDNIKGLQGCLSTIEIGGIYLSYFENVHGFINKPQEEQFLKISTNSVVTGCLQLNVCNSNPCLHGGNCEDIYSSYHCSCPLGWSGKHCELNIDECFSNPCIHGNCSDRVAAYHCTCEPGYTGVNCEVDIDNCQSHQCANGATCISHTNGYSCLCFGNFTGKFCRQSRLPSTVCGNEKTNLTCYNGGNCTEFQTELKCMCRPGFTGEWCEKDIDECASDPCVNGGLCQDLLNKFQCLCDVAFAGERCEVDLADDLISDIFTTIGSVTVALLLILLLAIVASVVTSNKRATQGTYSPSRQEKEGSRVEMWNLMPPPAMERLI

SEQ ID NO: 19 - protein, 1285 aa, Homo sapiens
  REGION 1..1285: note = MISC_FEATURE - CRB2 (protein crumbs homolog 2)
MALARPGTPDPQALASVLLLLLWAPALSLLAGTVPSEPPSACASDPCAPGTECQATESGGYTCGPMEPRGCATQPCHHGALCVPQGPDPTGFRCYCVPGFQGPRCELDIDECASRPCHHGATCRNLADRYECHCPLGYAGVTCEMEVDECASAPCLHGGSCLDGVGSFRCVCAPGYGGTRCQLDLDECQSQPCAHGGTCHDLVNGFRCDCAGTGYEGTHCEREVLECASAPCEHNASCLEGLGSFRCLCWPGYSGELCEVDEDECASSPCQHGGRCLQRSDPALYGGVQAAFPGAFSFRHAAGFLCHCPPGFEGADCGVEVDECASRPCLNGGHCQDLPNGFQCHCPDGYAGPTCEEDVDECLSDPCLHGGTCSDTVAGYICRCPETWGGRDCSVQLTGCQGHTCPLAATCIPIFESGVHSYVCHCPPGTHGPFCGQNTTFSVMAGSPIQASVPAGGPLGLALRFRTTLPAGTLATRNDTKESLELALVAATLQATLWSYSTTVLVLRLPDLALNDGHWHQVEVVLHLATLELRLWHEGCPARLCVASGPVALASTASATPLPAGISSAQLGDATFAGCLQDVRVDGHLLLPEDLGENVLLGCERREQCRPLPCVHGGSCVDLWTHFRCDCARPHRGPTCADEIPAATFGLGGAPSSASFLLQELPGPNLTVSFLLRTRESAGLLLQFANDSAAGLTVFLSEGRIRAEVPGSPAVVLPGRWDDGLRHLVMLSFGPDQLQDLGQHVHVGGRLLAADSQPWGGPFRGCLQDLRLDGCHLPFFPLPLDNSSQPSELGGRQSWNLTAGCVSEDMCSPDPCFNGGTCLVTWNDFHCTCPANFTGPTCAQQLWCPGQPCLPPATCEEVPDGFVCVAEATFREGPPAAFSGHNASSGRLLGGLSLAFRTRDSEAWLLRAAAGALEGVWLAVRNGSLAGGVRGGHGLPGAVLPIPGPRVADGAWHRVRLAMERPAATTSRWLLWLDGAATPVALRGLASDLGFLQGPGAVRILLAENFTGCLGRVALGGLPLPLARPRPGAAPGAREHFASWPGTPAPILGCRGAPVCAPSPCLHDGACRDLFDAFACACGPGWEGPRCEAHVDPCHSAPCARGRCHTHPDGRFECRCPPGFGGPRCRLPVPSKECSLNVTCLDGSPCEGGSPAANCSCLEGLAGQRCQVPTLPCEANPCLNGGTCRAAGGVSECICNARFSGQFCEVAKGLPLPLPFPLLEVAVPAACACLLLLLLGLLSGILAARKRRQSEGTYSPSQQEVAGARLEMDSVLKVPPEERLI

SEQ ID NO: 20 - protein, 299 aa, Homo sapiens [sequence omitted]

SEQ ID NO: 21 - protein, 6306 aa, Homo sapiens
  REGION 1..6306: note = MISC_FEATURE - GPR98 (G-protein coupled receptor 98)
MSVFLGPGMPSASLLVNLLSALLILFVFGETEIRFTGQTEFVVNETSTTVIRLIIERIGEPANVTAIVSLYGEDAGDFFDTYAAAFIPAGETNRTVYIAVCDDDLPEPDETFIFHLTLQKPSANVKLGWPRTVTVTILSNDNAFGIISFNMLPSIAVSEPKGRNESMPLTLIREKGTYGMVMVTFEVEGGPNPPDEDLSPVKGNITFPPGRATVIYNLTVLDDEVPENDEIFLIQLKSVEGGAEINTSRNSIEIIIKKNDSPVRFLQSIYLVPEEDHILIIPVVRGKDNNGNLIGSDEYEVSISYAVTTGNSTAHAQQNLDFIDLQPNTTVVFPPFIHESHLKFQIVDDTIPEIAESFHIMLLKDTLQGDAVLISPSVVQVTIKPNDKPYGVLSFNSVLFERTVIIDEDRISRYEEITVVRNGGTHGNVSANWVLTRNSTDPSPVTADIRPSSGVLHFAQGQMLATIPLTVVDDDLPEEAEAYLLQILPHTIRGGAEVSEPAELLFYIQDSDDVYGLITFFPMENQKIESSPGERYLSLSFTRLGGTKGDVRLLYSVLYIPAGAVDPLQAKEGILNISRRNDLIFPEQKTQVTTKLPIRNDAFLQNGAHFLVQLETVELLNIIPLIPPISPRFGEICNISLLVTPAIANGEIGFLSNLPIILHEPEDFAAEVVYIPLHRDGTDGQATVYWSLKPSGFNSKAVTPDDIGPFNGSVLFLSGQSDTTINITIKGDDIPEMNETVTLSLDRVNVENQVLKSGYTSRDLIILENDDPGGVFEFSPASRGPYVIKEGESVELHIIRSRGSLVKQFLHYRVEPRDSNEFYGNTGVLEFKPGEREIVITLLARLDGIPELDEHYWVVLSSHGERESKLGSATIVNITILKNDDPHGIIEFVSDGLIVMINESKGDAIYSAVYDVVRNRGNFGDVSVSWVVSPDFTQDVFPVQGTVVFGDQEFSKNITIYSLPDEIPEEMEEFTVILLNGTGGAKVGNRTTATLRIRRNDDPIYFAEPRVVRVQEGETANFTVLRNGSVDVTCMVQYATKDGKATARERDFIPVEKGETLIFEVGSRQQSISIFVNEDGIPETDEPFYIILLNSTGDTVVYQYGVATVIIEANDDPNGIFSLEPIDKAVEEGKTNAFWILRHRGYFGSVSVSWQLFQNDSALQPGQEFYETSGTVNFMDGEEAKPIILHAFPDKIPEFNEFYFLKLVNISGGSPGPGGQLAETNLQVTVMVPFNDDPFGVFILDPECLEREVAEDVLSEDDMSYITNFTILRQQGVFGDVQLGWEILSSEFPAGLPPMIDFLLVGIFPTTVHLQQHMRRHHSGTDALYFTGLEGAFGTVNPKYHPSRNNTIANFTFSAWVMPNANTNGFIIAKDDGNGSIYYGVKIQTNESHVTLSLHYKTLGSNATYIAKTTVMKYLEESVWLHLLIILEDGIIEFYLDGNAMPRGIKSLKGEAITDGPGILRIGAGINGNDRFTGLMQDVRSYERKLTLEEIYELHAMPAKSDLHPISGYLEFRQGETNKSFIISARDDNDEEGEELFILKLVSVYGGARISEENTTARLTIQKSDNANGLFGFTGACIPEIAEEGSTISCVVERTRGALDYVHVFYTISQIETDGINYLVDDFANASGTITFLPWQRSEVLNIYVLDDDIPELNEYFRVTLVSAIPGDGKLGSTPTSGASIDPEKETTDITIKASDHPYGLLQFSTGLPPQPKDAMTLPASSVPHITVEEEDGEIRLLVIRAQGLLGRVTAEFRTVSLTAFSPEDYQNVAGTLEFQPGERYKYIFINITDNSIPELEKSFKVELLNLEGGVAELFRVDGSGSGDGDMEFFLPTIHKRASLGVASQILVTIAASDHAHGVFEFSPESLFVSGTEPEDGYSTVTLNVIRHHGTLSPVTLHWNIDSDPDGDLAFTSGNITFEIGQTSANITVEILPDEDPELDKAFSVSVLSVSSGSLGAHINATLTVLASDDPYGIFIFSEKNRPVKVEEATQNITLSIIRLKGLMGKVLVSYATLDDMEKPPYFPPNLARATQGRDYIPASGFALFGANQSEATIAISILDDDEPERSESVFIELLNSTLVAKVQSRSIPNSPRLGPKVETIAQLIIIANDDAFGTLQLSAPIVRVAENHVGPIINVTRTGGAFADVSVKFKAVPITAIAGEDYSIASSDVVLLEGETSKAVPIYVINDIYPELEESFLVQLMNETTGGARLGALTEAVIIIEASDDPYGLFGFQITKLIVEEPEFNSVKVNLPIIRNSGTLGNVTVQWVATINGQLATGDLRVVSGNVTFAPGETIQTLLLEVLADDVPEIEEVIQVQLTDASGGGTIGLDRIANIIIPANDDPYGTVAFAQMVYRVQEPLERSSCANITVRRSGGHFGRLLLFYSTSDIDVVALAMEEGQDLLSYYESPIQGVPDPLWRTWMNVSAVGEPLYTCATLCLKEQACSAFSFFSASEGPQCFWMTSWISPAVNNSDFWTYRKNMTRVASLFSGQAVAGSDYEPVTRQWAIMQEGDEFANLTVSILPDDFPEMDESFLISLLEVHLMNISASLKNQPTIGQPNISTVVIALNGDAFGVFVIYNISPNTSEDGLFVEVQEQPQTLVELMIHRTGGSLGQVAVEWRVVGGTATEGLDFIGAGEILTFAEGETKKTVILTILDDSEPEDDESIIVSLVYTEGGSRILPSSDTVRVNILANDNVAGIVSFQTASRSVIGHEGEILQFHVIRTFPGRGNVTVNWKIIGQNLELNFANFSGQLFFPEGSLNTTLFVHLLDDNIPEEKEVYQVILYDVRTQGVPPAGIALLDAQGYAAVLTVEASDEPHGVLNFALSSRFVLLQEANITIQLFINREFGSLGAINVTYTTVPGMLSLKNQTVGNLAEPEVDFVPIIGFLILEEGETAAAINITILEDDVPELEEYFLVNLTYVGLTMAASTSFPPRLDSEGLTAQVIIDANDGARGVIEWQQSRFEVNETHGSLTLVAQRSREPLGHVSLFVYAQNLEAQVGLDYIFTPMILHFADGERYKNVNIMILDDDIPEGDEKFQLILTNPSPGLELGKNTIALIIVLANDDGPGVLSFNNSEHFFLREPTALYVQESVAVLYIVREPAQGLFGTVTVQFIVTEVNSSNESKDLTPSKGYIVLEEGVRFKALQISAILDTEPEMDEYFVCTLFNPTGGARLGVHVQTLITVLQNQAPLGLFSISAVENRATSIDIEEANRTVYLNVSRTNGIDLAVSVQWETVSETAFGMRGMDVVFSVFQSFLDESASGWCFFTLENLIYGIMLRKSSVTVYRWQGIFIPVEDLNIENPKTCEAFNIGFSPYFVITHEERNEEKPSLNSVFTFTSGFKLFLVQTIIILESSQVRYFTSDSQDYLIIASQRDDSELTQVFRWNGGSFVLHQKLPVRGVLTVALFNKGGSVFLAISQANARLNSLLFRWSGSGFINFQEVPVSGTTEVEALSSANDIYLIFAENVFLGDQNSIDIFIWEMGQSSFRYFQSVDFAAVNRIHSFTPASGIAHILLIGQDMSALYCWNSERNQFSFVLEVPSAYDVASVTVKSLNSSKNLIALVGAHSHIYELAYISSHSDFIPSSGELIFEPGEREATIAVNILDDTVPEKEESFKVQLKNPKGGAEIGINDSVTITILSNDDAYGIVAFAQNSLYKQVEEMEQDSLVTLNVERLKGTYGRITIAWEADGSISDIFPTSGVILFTEGQVLSTITLTILADNIPELSEVVIVTLTRITTEGVEDSYKGATIDQDRSKSVITTLPNDSPFGLVGWRAASVFIRVAEPKENTTTLQLQIARDKGLLGDIAIHLRAQPNFLLHVDNQATENEDYVLQETIIIMKENIKEAHAEVSILPDDLPELEEGFIVTITEVNLVNSDFSTGQPSVRRPGMEIAEIMIEENDDPRGIFMFHVTRGAGEVITAYEVPPPLNVLQVPVVRLAGSFGAVNVYWKASPDSAGLEDFKPSHGILEFADKQVTAMIEITIIDDAEFELTETFNISLISVAGGGRLGDDVVVTVVIPQNDSPFGVFGFEEKTVMIDESLSSDDPDSYVTLTVVRSPGGKGTVRLEWTIDEKAKHNLSPLNGTLHFDETESQKTIVLHTLQDTVLEEDRRFTIQLISIDEVEISPVKGSASIIIRGDKRASGEVGIAPSSRHILIGEPSAKYNGTAIISLVRGPGILGEVTVFWRIFPPSVGEFAETSGKLTMRDEQSAVIVVIQALNDDIPEEKSFYEFQLTAVSEGGVLSESSSTANITVVASDSPYGRFAFSHEQLRVSEAQRVNITIIRSSGDFGHVRLWYKTMSGTAEAGLDFVPAAGELLFEAGEMRKSLHVEILDDDYPEGPEEFSLTITKVELQGRGYDFTIQENGLQIDQPPEIGNISIVRIIIMKNDNAEGIIEFDPKYTAFEVEEDVGLIMIPVVRLHGTYGYVTADFISQSSSASPGGVDYILHGSTVTFQHGQNLSFINISIIDDNESEFEEPIEILLTGATGGAVLGRHLVSRIIIAKSDSPFGVIRFLNQSKISIANPNSTMILSLVLERTGGLLGEIQVNWETVGPNSQEALLPQNRDIADPVSGLFYFGEGEGGVRTIILTIYPHEEIEVEETFIIKLHLVKGEAKLDSRAKDVTLTIQEFGDPNGVVQFAPETLSKKTYSEPLALEGPLLITFFVRRVKGTFGEIMVYWELSSEFDITEDFLSTSGFFTIADGESEASFDVHLLPDEVPEIEEDYVIQLVSVEGGAELDLEKSITWFSVYANDDPHGVFALYSDRQSILIGQNLIRSIQINITRLAGTFGDVAVGLRISSDHKEQPIVTENAERQLVVKDGATYKVDVVPIKNQVFLSLGSNFTLQLVTVMLVGGRFYGMPTILQEAKSAVLPVSEKAANSQVGFESTAFQLMNITAGTSHVMISRRGTYGALSVAWTTGYAPGLEIPEFIVVGNMTPTLGSLSFSHGEQRKGVFLWTFPSPGWPEAFVLHLSGVQSSAPGGAQLRSGFIVAEIEPMGVFQFSTSSRNIIVSEDTQMIRLHVQRLFGFHSDLIKVSYQTTAGSAKPLEDFEPVQNGELFFQKFQTEVDFEITIINDQLSEIEEFFYINLTSVEIRGLQKFDVNWSPRLNLDFSVAVITILDNDDLAGMDISFPETTVAVAVDTTLIPVETESTTYLSTSKTTTILQPTNVVAIVTEATGVSAIPEKLVTLHGTPAVSEKPDVATVTANVSIHGTFSLGPSIVYIEEEMKNGTFNTAEVLIRRTGGFTGNVSITVKTFGERCAQMEPNALPFRGIYGISNLTWAVEEEDFEEQTLTLIFLDGERERKVSVQILDDDEPEGQEFFYVFLTNPQGGAQIVEEKDDTGFAAFAMVIITGSDLHNGIIGFSEESQSGLELREGAVMRRLHLIVTRQPNRAFEDVKVFWRVTLNKTVVVLQKDGVNLVEELQSVSGTTTCTMGQTKCFISIELKPEKVPQVEVYFFVELYEATAGAAINNSARFAQIKILESDESQSLVYFSVGSRLAVAHKKATLISLQVARDSGTGLMMSVNFSTQELRSAETIGRTIISPAISGKDFVITEGTLVFEPGQRSTVLDVILTPETGSLNSFPKRFQIVLFDPKGGARIDKVYGTANITLVSDADSQAIWGLADQLHQPVNDDILNRVLHTISMKVATENTDEQLSAMMHLIEKITTEGKIQAFSVASRTLFYEILCSLINPKRKDTRGFSHFAEVTENFAFSLLTNVTCGSPGEKSKTILDSCPYLSILALHWYPQQINGHKFEGKEGDYIRIPERLLDVQDAEIMAGKSTCKLVQFTEYSSQQWFISGNNLPTLKNKVLSLSVKGQSSQLLTNDNEVLYRIYAAEPRIIPQTSLCLLWNQAAASWLSDSQFCKVVEETADYVECACSHMSVYAVYARTDNLSSYNEAFFTSGFICISGLCLAVLSHIFCARYSMFAAKLLTHMMAASLGTQILFLASAYASPQLAEESCSAMAAVTHYLYLCQFSWMLIQSVNFWYVLVMNDEHTERRYLLFFLLSWGLPAFVVILLIVILKGIYHQSMSQIYGLIHGDLCFIPNVYAALFTAALVPLTCLVVVFVVFIHAYQVKPQWKAYDDVFRGRTNAAEIPLILYLFALISVTWLWGGLHMAYRHFWMLVLFVIFNSLQGLYVFMVYFILHNQMCCPMKASYTVEMNGHPGPSTAFFTPGSGMPPAGGEISKSTQNLIGAMEEVPPDWERASFQQGSQASPDLKPSPQNGATFPSSGGYGQGSLIADEESQEFDDLIFALKTGAGLSVSDNESGQGSQEGGTLTDSQIVELRRIPIADTHL

SEQ ID NO: 22 - protein, 201 aa, Homo sapiens
  REGION 1..201: note = MISC_FEATURE - GUCA1A (guanylyl cyclase-activating protein 1)
MGNVMEGKSVEELSSTECHQWYKKFMTECPSGQLTLYEFRQFFGLKNLSPSASQYVEQMFETFDFNKDGYIDFMEYVAALSLVLKGKVEQKLRWYFKLYDVDGNGCIDRDELLTIIQAIRAINPCSDTTMTAEEFTDTVFSKIDVNGDGELSLEEFIEGVQKDQMLLDTLTRSLDLTRIVRRLQNGEQDEEGADEAAEAAG

SEQ ID NO: 23 - protein, 200 aa, Homo sapiens
  REGION 1..200: note = MISC_FEATURE - GUCA1B (guanylyl cyclase-activating protein 2)
MGQEFSWEEAEAAGEIDVAELQEWYKKFVMECPSGTLFMHEFKRFFKVTDDEEASQYVEGMFRAFDKNGDNTIDFLEYVAALNLVLRGTLEHKLKWTFKIYDKDGNGCIDRLELLNIVEGIYQLKKACRRELQTEQGQLLTPEEVVDRIFLLVDENGDGQLSLNEFVEGARRDKWVMKMLQMDMNPSSWLAQQRRKSAMF

SEQ ID NO: 24 - protein, 2213 aa, Homo sapiens
  REGION 1..2213: note = MISC_FEATURE - MYO7A (unconventional myosin-VIIa), isoform 1
MVILQQGDHVWMDLRLGQEFDVPIGAVVKLCDSGQVQVVDDEDNEHWISPQNATHIKPMHPTSVHGVEDMIRLGDLNEAGILRNLLIRYRDHLIYTYTGSILVAVNPYQLLSIYSPEHIRQYTNKKIGEMPPHIFAIADNCYFNMKRNSRDQCCIISGESGAGKTESTKLILQFLAAISGQHSWIEQQVLEATPILEAFGNAKTIRNDNSSRFGKYIDIHFNKRGAIEGAKIEQYLLEKSRVCRQALDERNYHVFYCMLEGMSEDQKKKLGLGQASDYNYLAMGNCITCEGRVDSQEYANIRSAMKVLMFTDTENWEISKLLAAILHLGNLQYEARTFENLDACEVLFSPSLATAASLLEVNPPDLMSCLTSRTLITRGETVSTPLSREQALDVRDAFVKGIYGRLFVWIVDKINAAIYKPPSQDVKNSRRSIGLLDIFGFENFAVNSFEQLCINFANEHLQQFFVRHVFKLEQEEYDLESIDWLHIEFTDNQDALDMIANKPMNIISLIDEESKFPKGTDTTMLHKLNSQHKLNANYIPPKNNHETQFGINHFAGIVYYETQGFLEKNRDTLHGDIIQLVHSSRNKFIKQIFQADVAMGAETRKRSPTLSSQFKRSLELLMRTLGACQPFFVRCIKPNEFKKPMLFDRHLCVRQLRYSGMMETIRIRRAGYPIRYSFVEFVERYRVLLPGVKPAYKQGDLRGTCQRMAEAVLGTHDDWQIGKTKIFLKDHHDMLLEVERDKAITDRVILLQKVIRGFKDRSNFLKLKNAATLIQRHWRGHNCRKNYGLMRLGFLRLQALHRSRKLHQQYRLARQRIIQFQARCRAYLVRKAFRHRLWAVLTVQAYARGMIARRLHQRLRAEYLWRLEAEKMRLAEEEKLRKEMSAKKAKEEAERKHQERLAQLAREDAERELKEKEAARRKKELLEQMERARHEPVNHSDMVDKMFGFLGTSGGLPGQEGQAPSGFEDLERGRREMVEEDLDAALPLPDEDEEDLSEYKFAKFAATYFQGTTTHSYTRRPLKQPLLYHDDEGDQLAALAVWITILRFMGDLPEPKYHTAMSDGSEKIPVMTKIYETLGKKTYKRELQALQGEGEAQLPEGQKKSSVRHKLVHLTLKKKSKLTEEVTKRLHDGESTVQGNSMLEDRPTSNLEKLHFIIGNGILRPALRDEIYCQISKQLTHNPSKSSYARGWILVSLCVGCFAPSEKFVKYLRNFIHGGPPGYAPYCEERLRRTFVNGTRTQPPSWLELQATKSKKPIMLPVTFMDGTTKTLLTDSATTAKELCNALADKISLKDRFGFSLYIALFDKVSSLGSGSDHVMDAISQCEQYAKEQGAQERNAPWRLFFRKEVFTPWHSPSEDNVATNLIYQQVVRGVKFGEYRCEKEDDLAELASQQYFVDYGSEMILERLLNLVPTYIPDREITPLKTLEKWAQLAIAAHKKGIYAQRRTDAQKVKEDVVSYARFKWPLLFSRFYEAYKFSGPSLPKNDVIVAVNWTGVYFVDEQEQVLLELSFPEIMAVSSSRECRVWLSLGCSDLGCAAPHSGWAGLTPAGPCSPCWSCRGAKTTAPSFTLATIKGDEYTFTSSNAEDIRDLVVTFLEGLRKRSKYVVALQDNPNPAGEESGFLSFAKGDLIILDHDTGEQVMNSGWANGINERTKQRGDFPTDSVYVMPTVTMPPREIVALVTMTPDQRQDVVRLLQLRTAEPEVRAKPYTLEEFSYDYFRPPPKHTLSRVMVSKARGKDRLWSHTREPLKQALLKKLLGSEELSQEACLAFIAVLKYMGDYPSKRTRSVNELTDQIFEGPLKAEPLKDEAYVQILKQLTDNHIRYSEERGWELLWLCTGLFPPSNILLPHVQRFLQSRKHCPLAIDCLQRLQKALRNGSRKYPPHLVEVEAIQHKTTQIFHKVYFPDDTDEAFEVESSTKAKDFCQNIATRLLLKSSEGFSLFVKIADKVLSVPENDFFFDFVRHLTDWIKKARPIKDGIVPSLTYQVFFMKKLWTTTVPGKDPMADSIFHYYQELPKYLRGYHKCTREEVLQLGALIYRVKFEEDKSYFPSIPKLLRELVPQDLIRQVSPDDWKRSIVAYFNKHAGKSKEEAKLAFLKLIFKWPTFGSAFFEVKQTTEPNFPEILLIAINKYGVSLIDPKTKDILTTHPFTKISNWSSGNTYFHITIGNLVRGSKLLCETSLGYKMDDLLTSYISQMLTAMSKQRGSRS

SEQ ID NO: 25 - protein, 236 aa, Homo sapiens
  REGION 1..236: note = MISC_FEATURE - NRL (neural retina-specific leucine zipper protein), isoform 1
ALPPSPLAMEYVNDFDLMKFEVKREPSEGRPGPPTASLGSTPYSSVPPSPTFSEPGMVGATEGTRPGLEELYWLATLQQQLGAGEALGLSPEEAMELLQGQGPVPVDGPHGYYPGSPEETGAQHVQLAERFSDAALVSMSVRELNRQLRGCGRDEALRLKQRRRTLKNRGYAQACRSKRLQQRRGLEAERARLAAQLDALRAEVARLARERDLYKARCDRLTSSGPGSGDPSHLFL

SEQ ID NO: 26 - protein, 860 aa, Homo sapiens
  REGION 1..860: note = MISC_FEATURE - PDE6A (rod cGMP-specific 3',5'-cyclic phosphodiesterase subunit alpha)
MGEVTAEEVEKFLDSNIGFAKQYYNLHYRAKLISDLLGAKEAAVDFSNYHSPSSMEESEIIFDLLRDFQENLQTEKCIFNVMKKLCFLLQADRMSLFMYRTRNGIAELATRLFNVHKDAVLEDCLVMPDQEIVFPLDMGIVGHVAHSKKIANVPNTEEDEHFCDFVDILTEYKTKNILASPIMNGKDVVAIIMAVNKVDGSHFTKRDEEILLKYLNFANLIMKVYHLSYLHNCETRRGQILLWSGSKVFEELTDIERQFHKALYTVRAFLNCDRYSVGLLDMTKQKEFFDVWPVLMGEVPPYSGPRTPDGREINFYKVIDYILHGKEDIKVIPNPPPDHWALVSGLPAYVAQNGLICNIMNAPAEDFFAFQKEPLDESGWMIKNVLSMPIVNKKEEIVGVATFYNRKDGKPFDEMDETLMESLTQFLGWSVLNPDTYESMNKLENRKDIFQDIVKYHVKCDNEEIQKILKTREVYGKEPWECEEEELAEILQAELPDADKYEINKFHFSDLPLTELELVKCGIQMYYELKVVDKFHIPQEALVRFMYSLSKGYRKITYHNWRHGFNVGQTMFSLLVTGKLKRYFTDLEALAMVTAAFCHDIDHRGTNNLYQMKSQNPLAKLHGSSILERHHLEFGKTLLRDESLNIFQNLNRRQHEHAIHMMDIAIIATDLALYFKKRTMFQKIVDQSKTYESEQEWTQYMMLEQTRKEIVMAMMMTACDLSAITKPWEVQSQVALLVAAEFWEQGDLERTVLQQNPIPMMDRNKADELPKLQVGFIDFVCTFVYKEFSRFHEEITPMLDGITNNRKEWKALADEYDAKMKVQEEKKQKQQSAKSAAAGNQPGGNPSPGGATTSKSCCIQ

SEQ ID NO: 27 - protein, 854 aa, Homo sapiens
  REGION 1..854: note = MISC_FEATURE - PDE6B (rod cGMP-specific 3',5'-cyclic phosphodiesterase subunit beta), isoform 1
MSLSEEQARSFLDQNPDFARQYFGKKLSPENVAAACEDGCPPDCDSLRDLCQVEESTALLELVQDMQESINMERVVFKVLRRLCTLLQADRCSLFMYRQRNGVAELATRLFSVQPDSVLEDCLVPPDSEIVFPLDIGVVGHVAQTKKMVNVEDVAECPHFSSFADELTDYKTKNMLATPIMNGKDVVAVIMAVNKLNGPFFTSEDEDVFLKYLNFATLYLKIYHLSYLHNCETRRGQVLLWSANKVFEELTDIERQFHKAFYTVRAYLNCERYSVGLLDMTKEKEFFDVWSVLMGESQPYSGPRTPDGREIVFYKVIDYVLHGKEEIKVIPTPSADHWALASGLPSYVAESGFICNIMNASADEMFKFQEGALDDSGWLIKNVLSMPIVNKKEEIVGVATFYNRKDGKPFDEQDEVLMESLTQFLGWSVMNTDTYDKMNKLENRKDIAQDMVLYHVKCDRDEIQLILPTRARLGKEPADCDEDELGEILKEELPGPTTFDIYEFHFSDLECTELDLVKCGIQMYYELGVVRKFQIPQEVLVRFLFSISKGYRRITYHNWRHGFNVAQTMFTLLMTGKLKSYYTDLEAFAMVTAGLCHDIDHRGTNNLYQMKSQNPLAKLHGSSILERHHLEFGKFLLSEETLNIYQNLNRRQHEHVIHLMDIAIIATDLALYFKKRAMFQKIVDESKNYQDKKSWVEYLSLETTRKEIVMAMMMTACDLSAITKPWEVQSKVALLVAAEFWEQGDLERTVLDQQPIPMMDRNKAAELPKLQVGFIDFVCTFVYKEFSRFHEEILPMFDRLQNNRKEWKALADEYEAKVKALEEKEEEERVAAKKVGTEICNGGPAPKSSTCCIL

SEQ ID NO: 28 - protein, 346 aa, Homo sapiens
  REGION 1..346: note = MISC_FEATURE - PRPH2 (peripherin-2)
MALLKVKFDQKKRVKLAQGLWLMNWFSVLAGIIIFSLGLFLKIELRKRSDVMNNSESHFVPNSLIGMGVLSCVFNSLAGKICYDALDPAKYARWKPWLKPYLAICVLFNIILFLVALCCFLLRGSLENTLGQGLKNGMKYYRDTDTPGRCFMKKTIDMLQIEFKCCGNNGFRDWFEIQWISNRYLDFSSKEVKDRIKSNVDGRYLVDGVPFSCCNPSSPRPCIQYQITNNSAHYSYDHQTEELNLWVRGCRAALLSYYSSLMNSMGVVTLLIWLFEVTITIGLRYLQTSLDGVSNPEESESESEGWLLEKSVPETWKAFLESVKKLGKGNQVEAEGAGAGQAPEAG

SEQ ID NO: 29 - protein, 865 aa, Homo sapiens
  REGION 1..865: note = MISC_FEATURE - PROM1 (prominin-1), isoform 1
MALVLGSLLLLGLCGNSFSGGQPSSTDAPKAWNYELPATNYETQDSHKAGPIGILFELVHIFLYVVQPRDFPEDTLRKFLQKAYESKIDYDKPETVILGLKIVYYEAGIILCCVLGLLFIILMPLVGYFFCMCRCCNKCGGEMHQRQKENGPFLRKCFAISLLVICIIISIGIFYGFVANHQVRTRIKRSRKLADSNFKDLRTLLNETPEQIKYILAQYNTTKDKAFTDLNSINSVLGGGILDRLRPNIIPVLDEIKSMATAIKETKEALENMNSTLKSLHQQSTQLSSSLTSVKTSLRSSLNDPLCLVHPSSETCNSIRLSLSQLNSNPELRQLPPVDAELDNVNNVLRTDLDGLVQQGYQSLNDIPDRVQRQTTTVVAGIKRVLNSIGSDIDNVTQRLPIQDILSAFSVYVNNTESYIHRNLPTLEEYDSYWWLGGLVICSLLTLIVIFYYLGLLCGVCGYDRHATPTTRGCVSNTGGVFLMVGVGLSFLFCWILMIIVVLTFVFGANVEKLICEPYTSKELFRVLDTPYLLNEDWEYYLSGKLFNKSKMKLTFEQVYSDCKKNRGTYGTLHLQNSFNISEHLNINEHTGSISSELESLKVNLNIFLLGAAGRKNLQDFAACGIDRMNYDSYLAQTGKSPAGVNLLSFAYDLEAKANSLPPGNLRNSLKRDAQTIKTIHQQRVLPIEQSLSTLYQSVKILQRTGNGLLERVTRILASLDFAQNFITNNTSSVIIEETKKYGRTIIGYFEHYLQWIEFSISEKVASCKPVATALDTAVDVFLCSYIIDPLNLFWFGIGKATVFLLPALIFAVKLAKYYRRMDSEDVYDDVETIPMKNMENGNNGYHKDHVYGIHNPVMTSPSQH

SEQ ID NO: 30 - protein, 348 aa, Homo sapiens [sequence omitted]

SEQ ID NO: 31 - protein, 351 aa, Homo sapiens
  REGION 1..351: note = MISC_FEATURE - ROM1 (rod outer segment membrane protein 1)
MAPVLPLVLPLQPRIRLAQGLWLLSWLLALAGGVILLCSGHLLVQLRHLGTFLAPSCQFPVLPQAALAAGAVALGTGLVGVGASRASLNAALYPPWRGVLGPLLVAGTAGGGGLLVVGLGLALALPGSLDEALEEGLVTALAHYKDTEVPGHCQAKRLVDELQLRYHCCGRHGYKDWFGVQWVSSRYLDPGDRDVADRIQSNVEGLYLTDGVPFSCCNPHSPRPCLQNRLSDSYAHPLFDPRQPNQNLWAQGCHEVLLEHLQDLAGTLGSMLAVTFLLQALVLLGLRYLQTALEGLGGVIDAGGETQGYLFPSGLKDMLKTAWLQGGVACRPAPEEAPPGEAPPKEDLSEA

SEQ ID NO: 32 - protein, 2156 aa, Homo sapiens
  REGION 1..2156: note = MISC_FEATURE - RP1 (oxygen-regulated protein 1)
MSDTPSTGFSIIHPTSSEGQVPPPRHLSLTHPVVAKRISFYKSGDPQFGGVRVVVNPRSFKSFDALLDNLSRKVPLPFGVRNISTPRGRHSITRLEELEDGESYLCSHGRKVQPVDLDKARRRPRPWLSSRAISAHSPPHPVAVAAPGMPRPPRSLVVFRNGDPKTRRAVLLSRRVTQSFEAFLQHLTEVMQRPVVKLYATDGRRVPSLQAVILSSGAVVAAGREPFKPGNYDIQKYLLPARLPGISQRVYPKGNAKSESRKISTHMSSSSRSQIYSVSSEKTHNNDCYLDYSFVPEKYLALEKNDSQNLPIYPSEDDIEKSIIFNQDGTMTVEMKVRFRIKEEETIKWTTTVSKTGPSNNDEKSEMSFPGRTESRSSGLKLAACSFSADVSPMERSSNQEGSLAEEINIQMTDQVAETCSSASWENATVDTDIIQGTQDQAKHRFYRPPTPGLRRVRQKKSVIGSVTLVSETEVQEKMIGQFSYSEERESGENKSEYHMFTHSCSKMSSVSNKPVLVQINNNDQMEESSLERKKENSLLKSSAISAGVIEITSQKMLEMSHNNGLPSTISNNSIVEEDVVDCVVLDNKTGIKNFKTYGNTNDRFSPISADATHFSSNNSGTDKNISEAPASEASSTVTARIDRLINEFAQCGLTKLPKNEKKILSSVASKKKKKSRQQAINSRYQDGQLATKGILNKNERINTKGRITKEMIVQDSDSPLKGGILCEEDLQKSDTVIESNTFCSKSNLNSTISKNFHRNKLNTTQNSKVQGLLTKRKSRSLNKISLGAPKKREIGQRDKVFPHNESKYCKSTFENKSLFHVFNILEQKPKDFYAPQSQAEVASGYLRGMAKKSLVSKVTDSHITLKSQKKRKGDKVKASAILSKQHATTRANSLASLKKPDFPEAIAHHSIQNYIQSWLQNINPYPTLKPIKSAPVCRNETSVVNCSNNSFSGNDPHTNSGKISNFVMESNKHITKIAGLTGDNLCKEGDKSFIANDTGEEDLHETQVGSLNDAYLVPLHEHCTLSQSAINDHNTKSHIAAEKSGPEKKLVYQEINLARKRQSVEAAIQVDPIEEETPKDLLPVLMLHQLQASVPGIHKTQNGVVQMPGSLAGVPFHSAICNSSTNLLLAWLLVLNLKGSMNSFCQVDAHKATNKSSETLALLEILKHIAITEEADDLKAAVANLVESTTSHFGLSEKEQDMVPIDLSANCSTVNIQSVPKCSENERTQGISSLDGGCSASEACAPEVCVLEVTCSPCEMCTVNKAYSPKETCNPSDTFFPSDGYGVDQTSMNKACFLGEVCSLTDTVFSDKACAQKENHTYEGACPIDETYVPVNVCNTIDFLNSKENTYTDNLDSTEELERGDDIQKDLNILTDPEYKNGFNTLVSHQNVSNLSSCGLCLSEKEAELDKKHSSLDDFENCSLRKFQDENAYTSFDMEEPRTSEEPGSITNSMTSSERNISELESFEELENHDTDIFNTVVNGGEQATEELIQEEVEASKTLELIDISSKNIMEEKRMNGIIYEIISKRLATPPSLDFCYDSKQNSEKETNEGETKMVKMMVKTMETGSYSESSPDLKKCIKSPVTSDWSDYRPDSDSEQPYKTSSDDPNDSGELTQEKEYNIGFVKRAIEKLYGKADIIKPSFFPGSTRKSQVCPYNSVEFQCSRKASLYDSEGQSFGSSEQVSSSSSMLQEFQEERQDKCDVSAVRDNYCRGDIVEPGTKQNDDSRILTDIEEGVLIDKGKWLLKENHLLRMSSENPGMCGNADTTSVDTLLDNNSSEVPYSHFGNLAPGPTMDELSSSELEELTQPLELKCNYFNMPHGSDSEPFHEDLLDVRNETCAKERIANHHTEEKGSHQSERVCTSVTHSFISAGNKVYPVSDDAIKNQPLPGSNMIHGTLQEADSLDKLYALCGQHCPILTVIIQPMNEEDRGFAYRKESDIENFLGFYLWMKIHPYLLQTDKNVFREENNKASMRQNLIDNAIGDIFDQFYFSNTFDLMGKRRKQKRINFLGLEEEGNLKKFQPDLKERFCMNFLHTSLLVVGNVDSNTQDLSGQTNEIFKAVDENNNLLNNRFQGSRTNLNQVVRENINCHYFFEMLGQACLLDICQVETSLNISNRNILELCMFEGENLFIWEEEDILNLTDLESSREQEDL

SEQ ID NO: 33 - protein, 350 aa, Homo sapiens
  REGION 1..350: note = MISC_FEATURE - RP2 (protein XRP2)
MGCFFSKRRKADKESRPENEEERPKQYSWDQREKVDPKDYMFSGLKDETVGRLPGTVAGQQFLIQDCENCNIYIFDHSATVTIDDCTNCIIFLGPVKGSVFFRNCRDCKCTLACQQFRVRDCRKLEVFLCCATQPIIESSSNIKFGCFQWYYPELAFQFKDAGLSIFNNTWSNIHDFTPVSGELNWSLLPEDAVVQDYVPIPTTEELKAVRVSTEANRSIVPISRGQRQKSSDESCLVVLFAGDYTIANARKLIDEMVGKGFFLVQTKEVSMKAEDAQRVFREKAPDFLPLLNKGPVIALEFNGDGAVEVCQLIVNEIFNGTKMFVSESKETASGDVDSFYNFADIQMGI

SEQ ID NO: 34 - protein, 1020 aa, Homo sapiens
  REGION 1..1020: note = MISC_FEATURE - RPGR (X-linked retinitis pigmentosa GTPase regulator), isoform 1
MREPEELMPDSGAVFTFGKSKFAENNPGKFWFKNDVPVHLSCGDEHSAVVTGNNKLYMFGSNNWGQLGLGSKSAISKPTCVKALKPEKVKLAACGRNHTLVSTEGGNVYATGGNNEGQLGLGDTEERNTFHVISFFTSEHKIKQLSAGSNTSAALTEDGRLFMWGDNSEGQIGLKNVSNVCVPQQVTIGKPVSWISCGYYHSAFVTTDGELYVFGEPENGKLGLPNQLLGNHRTPQLVSEIPEKVIQVACGGEHTVVLTENAVYTFGLGQFGQLGLGTFLFETSEPKVIENIRDQTISYISCGENHTALITDIGLMYTFGDGRHGKLGLGLENFTNHFIPTLCSNFLRFIVKLVACGGCHMVVFAAPHRGVAKEIEFDEINDTCLSVATFLPYSSLTSGNVLQRTLSARMRRRERERSPDSFSMRRTLPPIEGTLGLSACFLPNSVFPRCSERNLQESVLSEQDLMQPEEPDYLLDEMTKEAEIDNSSTVESLGETTDILNMTHIMSLNSNEKSLKLSPVQKQKKQQTIGELTQDTALTENDDSDEYEEMSEMKEGKACKQHVSQGIFMTQPATTIEAFSDEEVGNDTGQVGPQADTDGEGLQKEVYRHENNNGVDQLDAKEIEKESDGGHSQKESEAEEIDSEKETKLAEIAGMKDLREREKSTKKMSPFFGNLPDRGMNTESEENKDFVKKRESCKQDVIFDSERESVEKPDSYMEGASESQQGIADGFQQPEAIEFSSGEKEDDEVETDQNIRYGRKLIEQGNEKETKPIISKSMAKYDFKCDRLSEIPEEKEGAEDSKGNGIEEQEVEANEENVKVHGGRKEKTEILSDDLTDKAEDHEFSKTEELKLEDVDEEINAENVESKKKTVGDDESVPTGYHSKTEGAERTNDDSSAETIEKKEKANLEERAICEYNENPKGYMLDDADSSSLEILENSETTPSKDMKKTKKIFLFKRVPSINQKIVKNNNEPLPEIKSIGDQIILKSDNKDADQNHMSQNHQNIPPTNTERRSKSCTIL

SEQ ID NO: 35 - protein, 405 aa, Homo sapiens
  REGION 1..405: note = MISC_FEATURE - SAG (S-arrestin)
MAASGKTSKSEPNHVIFKKISRDKSVTIYLGNRDYIDHVSQVQPVDGVVLVDPDLVKGKKVYVTLTCAFRYGQEDIDVIGLTFRRDLYFSRVQVYPPVGAASTPTKLQESLLKKLGSNTYPFLLTFPDYLPCSVMLQPAPQDSGKSCGVDFEVKAFATDSTDAEEDKIPKKSSVRLLIRKVQHAPLEMGPQPRAEAAWQFFMSDKPLHLAVSLNKEIYFHGEPIPVTVTVTNNTEKTVKKIKAFVEQVANVVLYSSDYYVKPVAMEEAQEKVPPNSTLTKTLTLLPLLANNRERRGIALDGKIKHEDTNLASSTIIKEGIDRTVLGILVSYQIKVKLTVSGFLGELTSSEVATEVPFRLMHPQPEDPAKESYQDANLVFEEFARHNLKDAGEAEEGKRDKNDVDE

SEQ ID NO: 36 - protein, 552 aa, Homo sapiens
  REGION 1..552: note = MISC_FEATURE - USH1C (Usher syndrome type-1 C protein), isoform 1
MDRKVAREFRHKVDFLIENDAEKDYLYDVLRMYHQTMDVAVLVGDLKLVINEPSRLPLFDAIRPLIPLKHQVEYDQLTPRRSRKLKEVRLDRLHPEGLGLSVRGGLEFGCGLFISHLIKGGQADSVGLQVGDEIVRINGYSISSCTHEEVINLIRTKKTVSIKVRHIGLIPVKSSPDEPLTWQYVDQFVSESGGVRGSLGSPGNRENKEKKVFISLVGSRGLGCSISSGPIQKPGIFISHVKPGSLSAEVGLEIGDQIVEVNGVDFSNLDHKEAVNVLKSSRSLTISIVAAAGRELFMTDRERLAEARQRELQRQELLMQKRLAMESNKILQEQQEMERQRRKEIAQKAAEENERYRKEMEQIVEEEEKFKKQWEEDWGSKEQLLLPKTITAEVHPVPLRKPKYDQGVEPELEPADDLDGGTEEQGEQDFRKYEEGFDPYSMFTPEQIMGKDVRLLRIKKEGSLDLALEGGVDSPIGKVVVSAVYERGAAERHGGIVKGDEIMAINGKIVTDYTLAEAEAALQKAWNQGGDWIDLVVAVCPPKEYDDELTFF

SEQ ID NO: 37 - protein, 461 aa, Homo sapiens
  REGION 1..461: note = MISC_FEATURE - USH1G (Usher syndrome type-1G protein)
MNDQYHRAARDGYLELLKEATRKELNAPDEDGMTPTLWAAYHGNLESLRLIVSRGGDPDKCDIWGNTPLHLAASNGHLHCLSFLVSFGANIWCLDNDYHTPLDMAAMKGHMECVRYLDSIAAKQSSLNPKLVGKLKDKAFREAERRIRECAKLQRRHHERMERRYRRELAERSDTLSFSSLTSSTLSRRLQHLALGSHLPYSQATLHGTARGKTKMQKKLERRKQGGEGTFKVSEDGRKSARSLSGLQLGSDVMFVRQGTYANPKEWGRAPLRDMFLSDEDSVSRATLAAEPAHSEVSTDSGHDSLFTRPGLGTMVFRRNYLSSGLHGLGREDGGLDGVGAPRGRLQSSPSLDDDSLGSANSLQDRSCGEELPWDELDLGLDEDLEPETSPLETFLASLHMEDFAALLRQEKIDLEALMLCSDLDLRSISVPLGPRKKILGAVRRRRQAMERPPALEDTEL

SEQ ID NO: 38 - protein, 5202 aa, Homo sapiens
  REGION 1..5202: note = MISC_FEATURE - USH2A (Usherin), isoform 1
MNCPVLSLGSGFLFQVIEMLIFAYFASISLTESRGLFPRLENVGAFKKVSIVPTQAVCGLPDRSTFCHSSAAAESIQFCTQRFCIQDCPYRSSHPTYTALFSAGLSSCITPDKNDLHPNAHSNSASFIFGNHKSCFSSPPSPKLMASFTLAVWLKPEQQGVMCVIEKTVDGQIVFKLTISEKETMFYYRTVNGLQPPIKVMTLGRILVKKWIHLSVQVHQTKISFFINGVEKDHTPFNARTLSGSITDFASGTVQIGQSLNGLEQFVGRMQDFRLYQVALTNREILEVFSGDLLRLHAQSHCRCPGSHPRVHPLAQRYCIPNDAGDTADNRVSRLNPEAHPLSFVNDNDVGTSWVSNVFTNITQLNQGVTISVDLENGQYQVFYIIIQFFSPQPTEIRIQRKKENSLDWEDWQYFARNCGAFGMKNNGDLEKPDSVNCLQLSNFTPYSRGNVTFSILTPGPNYRPGYNNFYNTPSLQEFVKATQIRFHFHGQYYTTETAVNLRHRYYAVDEITISGRCQCHGHADNCDTTSQPYRCLCSQESFTEGLHCDRCLPLYNDKPFRQGDQVYAFNCKPCQCNSHSKSCHYNISVDPFPFEHFRGGGGVCDDCEHNTTGRNCELCKDYFFRQVGADPSAIDVCKPCDCDTVGTRNGSILCDQIGGQCNCKRHVSGRQCNQCQNGFYNLQELDPDGCSPCNCNTSGTVDGDITCHQNSGQCKCKANVIGLRCDHCNFGFKFLRSFNDVGCEPCQCNLHGSVNKFCNPHSGQCECKKEAKGLQCDTCRENFYGLDVTNCKACDCDTAGSLPGTVCNAKTGQCICKPNVEGRQCNKCLEGNFYLRQNNSFLCLPCNCDKTGTINGSLLCNKSTGQCPCKLGVTGLRCNQCEPHRYNLTIDNFQHCQMCECDSLGTLPGTICDPISGQCLCVPNRQGRRCNQCQPGFYISPGNATGCLPCSCHTTGAVNHICNSLTGQCVCQDASIAGQRCDQCKDHYFGFDPQTGRCQPCNCHLSGALNETCHLVTGQCFCKQFVTGSKCDACVPSASHLDVNNLLGCSKTPFQQPPPRGQVQSSSAINLSWSPPDSPNAHWLTYSLLRDGFEIYTTEDQYPYSIQYFLDTDLLPYTKYSYYIETTNVHGSTRSVAVTYKTKPGVPEGNLTLSYIIPIGSDSVTLTWTTLSNQSGPIEKYILSCAPLAGGQPCVSYEGHETSATIWNLVPFAKYDFSVQACTSGGCLHSLPITVTTAQAPPQRLSPPKMQKISSTELHVEWSPPAELNGIIIRYELYMRRLRSTKETTSEESRVFQSSGWLSPHSFVESANENALKPPQTMTTITGLEPYTKYEFRVLAVNMAGSVSSAWVSERTGESAPVFMIPPSVFPLSSYSLNISWEKPADNVTRGKVVGYDINMLSEQSPQQSIPMAFSQLLHTAKSQELSYTVEGLKPYRIYEFTITLCNSVGCVTSASGAGQTLAAAPAQLRPPLVKGINSTTIHLRWFPPEELNGPSPIYQLERRESSLPALMTTMMKGIRFIGNGYCKFPSSTHPVNTDFTGIKASFRTKVPEGLIVFAASPGNQEEYFALQLKKGRLYFLFDPQGSPVEVTTTNDHGKQYSDGKWHEIIAIRHQAFGQITLDGIYTGSSAILNGSTVIGDNTGVFLGGLPRSYTILRKDPEIIQKGFVGCLKDVHFMKNYNPSAIWEPLDWQSSEEQINVYNSWEGCPASLNEGAQFLGAGFLELHPYMFHGGMNFEISFKFRTDQLNGLLLFVYNKDGPDFLAMELKSGILTFRLNTSLAFTQVDLLLGLSYCNGKWNKVIIKKEGSFISASVNGLMKHASESGDQPLVVNSPVYVGGIPQELLNSYQHLCLEQGFGGCMKDVKFTRGAVVNLASVSSGAVRVNLDGCLSTDSAVNCRGNDSILVYQGKEQSVYEGGLQPFTEYLYRVIASHEGGSVYSDWSRGRTTGAAPQSVPTPSRVRSLNGYSIEVTWDEPVVRGVIEKYILKAYSEDSTRPPRMPSASAEFVNTSNLTGILTGLLPFKNYAVTLTACTLAGCTESSHALNISTPQEAPQEVQPPVAKSLPSSLLLSWNPPKKANGIITQYCLYMDGRLIYSGSEENYIVTDLAVFTPHQFLLSACTHVGCTNSSWVLLYTAQLPPEHVDSPVLTVLDSRTIHIQWKQPRKISGILERYVLYMSNHTHDFTIWSVIYNSTELFQDHMLQYVLPGNKYLIKLGACTGGGCTVSEASEALTDEDIPEGVPAPKAHSYSPDSFNVSWTEPEYPNGVITSYGLYLDGILIHNSSELSYRAYGFAPWSLHSFRVQACTAKGCALGPLVENRTLEAPPEGTVNVFVKTQGSRKAHVRWEAPFRPNGLLTHSVLFTGIFYVDPVGNNYTLLNVTKVMYSGEETNLWVLIDGLVPFTNYTVQVNISNSQGSLITDPITIAMPPGAPDGVLPPRLSSATPTSLQVVWSTPARNNAPGSPRYQLQMRSGDSTHGFLELFSNPSASLSYEVSDLQPYTEYMFRLVASNGFGSAHSSWIPFMTAEDKPGPVVPPILLDVKSRMMLVTWQHPRKSNGVITHYNIYLHGRLYLRTPGNVTNCTVMHLHPYTAYKFQVEACTSKGCSLSPESQTVWTLPGAPEGIPSPELFSDTPTSVIISWQPPTHPNGLVENFTIERRVKGKEEVTTLVTLPRSHSMRFIDKTSALSPWTKYEYRVLMSTLHGGTNSSAWVEVTTRPSRPAGVQPPVVTVLEPDAVQVTWKPPLIQNGDILSYEIHMPDPHITLTNVTSAVLSQKVTHLIPFTNYSVTIVACSGGNGYLGGCTESLPTYVTTHPTVPQNVGPLSVIPLSESYVVISWQPPSKPNGPNLRYELLRRKIQQPLASNPPEDLNRWHNIYSGTQWLYEDKGLSRFTTYEYMLFVHNSVGFTPSREVTVTTLAGLPERGANLTASVLNHTAIDVRWAKPTVQDLQGEVEYYTLFWSSATSNDSLKILPDVNSHVIGHLKPNTEYWIFISVFNGVHSINSAGLHATTCDGEPQGMLPPEVVIINSTAVRVIWTSPSNPNGVVTEYSIYVNNKLYKTGMNVPGSFILRDLSPFTIYDIQVEVCTIYACVKSNGTQITTVEDTPSDIPTPTIRGITSRSLQIDWVSPRKPNGIILGYDLLWKTWYPCAKTQKLVQDQSDELCKAVRCQKPESICGHICYSSEAKVCCNGVLYNPKPGHRCCEEKYIPFVLNSTGVCCGGRIQEAQPNHQCCSGYYARILPGEVCCPDEQHNRVSVGIGDSCCGRMPYSTSGNQICCAGRLHDGHGQKCCGRQIVSNDLECCGGEEGVVYNRLPGMFCCGQDYVNMSDTICCSASSGESKAHIKKNDPVPVKCCETELIPKSQKCCNGVGYNPLKYVCSDKISTGMMMKETKECRILCPASMEATEHCGRCDFNFTSHICTVIRGSHNSTGKASIEEMCSSAEETIHTGSVNTYSYTDVNLKPYMTYEYRISAWNSYGRGLSKAVRARTKEDVPQGVSPPTWTKIDNLEDTIVLNWRKPIQSNGPIIYYILLRNGIERFRGTSLSFSDKEGIQPFQEYSYQLKACTVAGCATSSKVVAATTQGVPESILPPSITALSAVALHLSWSVPEKSNGVIKEYQIRQVGKGLIHTDTTDRRQHTVTGLQPYTNYSFTLTACTSAGCTSSEPFLGQTLQAAPEGVWVTPRHIIINSTTVELYWSLPEKPNGLVSQYQLSRNGNLLFLGGSEEQNFTDKNLEPNSRYTYKLEVKTGGGSSASDDYIVQTPMSTPEEIYPPYNITVIGPYSIFVAWIPPGILIPEIPVEYNVLLNDGSVTPLAFSVGHHQSTLLENLTPFTQYEIRIQACQNGSCGVSSRMFVKTPEAAPMDLNSPVLKALGSACIEIKWMPPEKPNGIIINYFIYRRPAGIEEESVLFVWSEGALEFMDEGDTLRPFTLYEYRVRACNSKGSVESLWSLTQTLEAPPQDFPAPWAQATSAHSVLLNWTKPESPNGIISHYRVVYQERPDDPTFNSPTVHAFTVKGTSHQAHLYGLEPFTTYRIGVVAANHAGEILSPWTLIQTLESSPSGLRNFIVEQKENGRALLLQWSEPMRTNGVIKTYNIFSDGFLEYSGLNRQFLFRRLDPFTLYTLTLEACTRAGCAHSAPQPLWTDEAPPDSQLAPTVHSVKSTSVELSWSEPVNPNGKIIRYEVIRRCFEGKAWGNQTIQADEKIVFTEYNTERNTFMYNDTGLQPWTQCEYKIYTWNSAGHTCSSWNVVRTLQAPPEGLSPPVISYVSMNPQKLLISWIPPEQSNGIIQSYRLQRNEMLYPFSFDPVTFNYTDEELLPFSTYSYALQACTSGGCSTSKPTSITTLEAAPSEVSPPDLWAVSATQMNVCWSPPTVQNGKITKYLVRYDNKESLAGQGLCLLVSHLQPYSQYNFSLVACTNGGCTASVSKSAWTMEALPENMDSPTLQVTGSESIEITWKPPRNPNGQIRSYELRRDGTIVYTGLETRYRDFTLTPGVEYSYTVTASNSQGGILSPLVKDRTSPSAPSGMEPPKLQARGPQEILVNWDPPVRTNGDIINYTLFIRELFERETKIIHINTTHNSFGMQSYIVNQLKPFHRYEIRIQACTTLGCASSDWTFIQTPEIAPLMQPPPHLEVQMAPGGFQPTVSLLWTGPLQPNGKVLYYELYRRQIATQPRKSNPVLIYNGSSTSFIDSELLPFTEYEYQVWAVNSAGKAPSSWTWCRTGPAPPEGLRAPTFHVISSTQAVVNISAPGKPNGIVSLYRLFSSSAHGAETVLSEGMATQQTLHGLQAFTNYSIGVEACTCFNCCSKGPTAELRTHPAPPSGLSSPQIGTLASRTASFRWSPPMFPNGVIHSYELQFHVACPPDSALPCTPSQIETKYTGLGQKASLGGLQPYTTYKLRVVAHNEVGSTASEWISFTTQKELPQYRAPFSVDSNLSVVCVNWSDTFLLNGQLKEYVLTDGGRRVYSGLDTTLYIPRTADKTFFFQVICTTDEGSVKTPLIQYDTSTGLGLVLTTPGKKKGSRSKSTEFYSELWFIVLMAMLGLILLAIFLSLILQRKIHKEPYIRERPPLVPLQKRMSPLNVYPPGENHMGLADTKIPRSGTPVSIRSNRSACVLRIPSQNQTSLTYSQGSLHRSVSQLMDIQDKKVLMDNSLWEAIMGHNSGLYVDEEDLMNAIKDFSSVTKERTTFTDTHL

SEQ ID NO: 39 - protein, 410 aa, Homo sapiens
  REGION 1..410: note = MISC_FEATURE - NR2E3 (photoreceptor-specific nuclear receptor), isoform long
METRPTALMSSTVAAAAPAAGAASRKESPGRWGLGEDPTGVSPSLQCRVCGDSSSGKHYGIYACNGCSGFFKRSVRRRLIYRCQVGAGMCPVDKAHRNQCQACRLKKCLQAGMNQDAVQNERQPRSTAQVHLDSMESNTESRPESLVAPPAPAGRSPRGPTPMSAARALGHHFMASLITAETCAKLEPEDADENIDVTSNDPEFPSSPYSSSSPCGLDSIHETSARLLFMAVKWAKNLPVFSSLPFRDQVILLEEAWSELFLLGAIQWSLPLDSCPLLAPPEASAAGGAQGRLTLASMETRVLQETISRFRALAVDPTEFACMKALVLFKPETRGLKDPEHVEALQDQSQVMLSQHSKAHHPSQPVRFGKLLLLLPSLRFITAERIELLFFRKTIGNTPMEKLLCDMFKN

SEQ ID NO: 40 - protein, 1250 aa, Homo sapiens
  REGION 1..1250: note = MISC_FEATURE - human CNGB1 protein (NGS)
MLGWVQRVLPQPPGTPRKTKMQEEEEVEPEPEMEAEVEPEPNPEEAETESESMPPEESFKEEEVAVADPSPQETKEAALTSTISLRAQGAEISEMNSPSHRVLTWLMKGVEKVIPQPVHSITEDPAQILGHGSTGDTGCTDEPNEALEAQDTRPGLRLLLWLEQNLERVLPQPPKSSEVWRDEPAVATGAASDPAPPGRPQEMGPKLQARETPSLPTPIPLQPKEEPKEAPAPEPQPGSQAQTSSLPPTRDPARLVAWVLHRLEMALPQPVLHGKIGEQEPDSPGICDVQTISILPGGQVEPDLVLEEVEPPWEDAHQDVSTSPQGTEVVPAYEEENKAVEKMPRELSRIEEEKEDEEEEEEEEEEEEEEEVTEVLLDSCVVSQVGVGQSEEDGTRPQSTSDQLWEEVGEEAKKEAEEKAKEEAEEVAEEEAEKEPQDWAETKEEPEAEAEAASSGVPATKQHPEVQVEDTDADSCPLMAEENPPSTVLPPPSPAKSDTLIVPSSASGTHRKKLPSEDDEAEELKALSPAESPVVAWSDPTTPKDTDGQDRAASTASTNSAIINDRLQELVKLFKERTEKVKEKLIDPDVTSDEESPKPSPAKKAPEPAPDTKPAEAEPVEEEHYCDMLCCKFKHRPWKKYQFPQSIDPLTNLMYVLWLFFVVMAWNWNCWLIPVRWAFPYQTPDNIHHWLLMDYLCDLIYFLDITVFQTRLQFVRGGDIITDKKDMRNNYLKSRRFKMDLLSLLPLDFLYLKVGVNPLLRLPRCLKYMAFFEFNSRLESILSKAYVYRVIRTTAYLLYSLHLNSCLYYWASAYQGLGSTHWVYDGVGNSYIRCYYFAVKTLITIGGLPDPKTLFEIVFQLLNYFTGVFAFSVMIGQMRDVVGAATAGQTYYRSCMDSTVKYMNFYKIPKSVQNRVKTWYEYTWHSQGMLDESELMVQLPDKMRLDLAIDVNYNIVSKVALFQGCDRQMIFDMLKRLRSVVYLPNDYVCKKGEIGREMYIIQAGQVQVLGGPDGKSVLVTLKAGSVFGEISLLAVGGGNRRTANVVAHGFTNLFILDKKDLNEILVHYPESQKLLRKKARRMLRSNNKPQEEKSVLILPPRAGTPKLFNAALAMTGKMGGKGAKGGKLAHLRARLKELAALEAAAKQQELVEQAKSSQDVKGEEGSAAPDQHTHPKEAATDPPAPRTPPEPPGSPPSSPPPASLGRPEGEEEGPAEPEEHSVRICMSPGPEPGEQILSVKMPEEREEKAE

SEQ ID NO: 41 - protein, 1251 aa, Homo sapiens
  REGION 1..1251: note = MISC_FEATURE - human CNGB1 protein (GenBank)
MLGWVQRVLPQPPGTPRKTKMQEEEEVEPEPEMEAEVEPEPNPEEAETESESMPPEESFKEEEVAVADPSPQETKEAALTSTISLRAQGAEISEMNSPSRRVLTWLMKGVEKVIPQPVHSITEDPAQILGHGSTGDTGCTDEPNEALEAQDTRPGLRLLLWLEQNLERVLPQPPKSSEVWRDEPAVATGAASDPAPPGRPQEMGPKLQARETPSLPTPIPLQPKEEPKEAPAPEPQPGSQAQTSSLPPTRDPARLVAWVLHRLEMALPQPVLHGKIGEQEPDSPGICDVQTISILPGGQVEPDLVLEEVEPPWEDAHQDVSTSPQGTEVVPAYEEENKAVEKMPRELSRIEEEKEDEEEEEEEEEEEEEEEVTEVLLDSCVVSQVGVGQSEEDGTRPQSTSDQKLWEEVGEEAKKEAEEKAKEEAEEVAEEEAEKEPQDWAETKEEPEAEAEAASSGVPATKQHPEVQVEDTDADSCPLMAEENPPSTVLPPPSPAKSDTLIVPSSASGTHRKKLPSEDDEAEELKALSPAESPVVAWSDPTTPKDTDGQDRAASTASTNSAIINDRLQELVKLFKERTEKVKEKLIDPDVTSDEESPKPSPAKKAPEPAPDTKPAEAEPVEEEHYCDMLCCKFKHRPWKKYQFPQSIDPLTNLMYVLWLFFVVMAWNWNCWLIPVRWAFPYQTPDNIHHWLLMDYLCDLIYFLDITVFQTRLQFVRGGDIITDKKDMRNNYLKSRRFKMDLLSLLPLDFLYLKVGVNPLLRLPRCLKYMAFFEFNSRLESILSKAYVYRVIRTTAYLLYSLHLNSCLYYWASAYQGLGSTHWVYDGVGNSYIRCYYFAVKTLITIGGLPDPKTLFEIVFQLLNYFTGVFAFSVMIGQMRDVVGAATAGQTYYRSCMDSTVKYMNFYKIPKSVQNRVKTWYEYTWHSQGMLDESELMVQLPDKMRLDLAIDVNYNIVSKVALFQGCDRQMIFDMLKRLRSVVYLPNDYVCKKGEIGREMYIIQAGQVQVLGGPDGKSVLVTLKAGSVFGEISLLAVGGGNRRTANVVAHGFTNLFILDKKDLNEILVHYPESQKLLRKKARRMLRSNNKPKEEKSVLILPPRAGTPKLFNAALAMTGKMGGKGAKGGKLAHLRARLKELAALEAAAKQQELVEQAKSSQDVKGEEGSAAPDQHTHPKEAATDPPAPRTPPEPPGSPPSSPPPASLGRPEGEEEGPAEPEEHSVRICMSPGPEPGEQILSVKMPEEREEKAE

SEQ ID NO: 42 - DNA, 4651 bp, synthetic construct
  misc_feature 1..4651: note = 5'ITR-hRHO promoter-CNGB1a-SV40polyA-3'ITR
  source 1..4651: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagcctctcctccctgacctcaggcttcctcctagtgtcaccttggcccctcttagaagccaattaggccctcagtttctgcagcggggattaatatgattatgaacacccccaatctcccagatgctgattcagccaggagcttaggagggggaggtcactttataagggtctgggggggtcagaacccagagtcatcactagtaacggccgccagtgtgctggaattcgcccttctccaccgccatgttgggctgggtccagagggtgctgcctcagcccccagggacccctcggaagaccaagatgcaggaggaagaggaagtggaaccagagccagagatggaggcggaggtggaaccagaaccgaatcctgaggaggccgagacagagtccgagtccatgccccccgaagagtcattcaaggaggaggaagtggctgtggcagacccaagccctcaggagaccaaggaggctgcccttacttccaccatatccctccgggcccagggcgctgagatttctgaaatgaatagtcccagccacagggtactgacctggctcatgaagggtgtagagaaggtgatcccgcagcctgttcacagcatcacggaggacccggctcagatcctggggcatggcagcactggggacacagggtgcacagatgaacccaatgaggcccttgaggcccaagacactaggcctgggctgcggctgcttctgtggctggagcagaatctggaaagagtgcttcctcagccccccaaatcctctgaggtctggagagatgagcctgcagttgctacagcgcctccaggacgcccccaggaaatggggcccaagctgcaggcccgggagaccccctccctgcccacacccatccccctgcagcccaaggaggaacccaaggaggcaccagctccagagccccagcccggctcccaggcccagacctcctccctgccaccaaccagggaccctgccaggctggtggcatgggtcctgcacaggctggagatggccttgccgcagccagtgctacatgggaaaataggggaacaggagcctgactcccctgggatatgtgatgtgcagaccatcagcatccttcctggaggacaagtggagcctgaccttgtcctagaggaggttgaaccgccctgggaggatgcccaccaggatgtcagtaccagcccacagggtacagaggtggttccagcttatgaagaagagaacaaagctgtggagaagatgcccagagagctgtcccggattgaagaggagaaagaagatgaggaggaggaagaggaagaggaggaggaggaggaagaggaggaggtgactgaggtgctgctggatagctgtgtggtgtcgcaggtgggcgtgggccagagtgaagaagacgggacccggccccagagcacttcagatcagaagctgtgggaggaagttggggaggaggccaagaaggaggctgaagagaaggccaaggaggaggccgaggaggtggctgaagaggaggctgaaaaggagccccaggactgggcggagaccaaggaggagcctgaggctgaggccgaggctgccagttcaggagtgcctgccacgaaacagcacccagaagtgcaggtggaagatactgatgctgatagctgccccctcatggcagaagagaatccaccctcaaccgtgttgccgccaccatctcctgccaaatcagacacccttatagtcccaagctcagcctcggggacacacaggaagaagctgccctctgaggatgatgaggctgaagagctcaaggcgttgtcaccagcagagtccccagtggttgcctggtctgaccccaccaccccgaaggacactgatggccaggaccgtgcggcctccacggccagcacaaatagcgccatcatcaacgaccggctccaggagctggtgaagctcttcaaggagcggacagagaaagtgaaggagaaactcattgaccctgacgtcacctctgatgaggagagccccaagccctccccagccaagaaagccccagagccagctccagacacaaagcccgctgaagccgagccagtggaagaggagcactattgcgacatgctctgctgcaagttcaaacaccgcccctggaagaagtaccagtttccccagagcattgacccgctgaccaacctgatgtatgtcctatggctgttcttcgtggtgatggcctggaattggaactgttggctgattcccgtgcgctgggccttcccctaccagaccccggacaacatccaccactggctgctgatggattacctatgcgacctcatctacttcctggacatcaccgtgttccagacacgcctgcagtttgtcagaggcggggacatcattacggacaaaaaggacatgcgaaataactacctgaagtctcgccgcttcaagatggacctgctcagcctcctgcccttggattttctctatttgaaagtcggtgtgaaccccctcctccgcctgccccgctgtttaaagtacatggccttcttcgagtttaacagccgcctggaatccatcctcagcaaagcctacgtgtacagggtcatcaggaccacagcctaccttctctacagcctgcatttgaattcctgtctttattactgggcatcggcctatcagggcctcggctccactcactgggtttacgatggcgtgggaaacagttatattcgctgttactactttgctgtgaagaccctcatcaccatcggggggctgcctgaccccaagacactctttgaaattgtcttccagctgctgaattatttcacgggcgtctttgctttctctgtgatgatcggacagatgagagatgtggtaggggccgccaccgcgggacagacctactaccgcagctgcatggacagcacggtgaagtacatgaatttctacaagatccccaagtccgtgcagaaccgcgtcaagacctggtacgagtacacctggcactcgcaaggcatgctggatgagtcagagctgatggtgcagcttccagacaagatgcggctggacctcgccatcgacgtgaactacaacatcgttagcaaagtcgcactctttcagggctgtgaccggcagatgatctttgacatgctgaagaggcttcgctctgttgtctacctgcccaacgactatgtgtgcaagaagggggagatcggccgtgagatgtacatcatccaggcagggcaagtgcaggtcttgggcggccctgatgggaaatctgtgctggtgacgctgaaagctggatctgtgtttggagaaataagcttgctggctgttgggggcgggaaccggcgcacggccaacgtggtggcgcacgggtttaccaacctcttcatcctggataagaaggacctgaatgagattttggtgcattatcctgagtctcagaagttactccggaagaaagccaggcgcatgctgagaagcaacaataagcccaaggaggagaagagcgtgctgatccttccaccccgggcgggcaccccaaagctcttcaacgctgccctcgctatgacaggaaagatgggtggcaagggggcaaaaggcggcaaacttgctcacctccgggcccggctcaaagaactggccgcgctggaggcggctgcaaagcagcaagagttggtggaacaggccaagagctcgcaagacgtcaagggagaggaaggctccgccgccccagaccagcacacgcacccaaaggaggccgccaccgacccacccgcgccccggacgccccccgagcccccggggtctccaccgagctctccaccgcctgcctcccttgggaggccggagggagaggaggaggggccggccgagcccgaagagcactcggtgaggatctgcatgagcccgggcccggagccgggagagcagatcctgtcggtgaagatgccggaggaaagggaggagaaggcggagtaaggtggggtgaggcggatccatggccgcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtactcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 43 - DNA, 4665 bp, synthetic construct
  misc_feature 1..4665: note = 5'ITR-hRHO promoter-CNGB1a-SV40polyA-3'ITR (NGS)
  source 1..4665: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagcctctcctccctgacctcaggcttcctcctagtgtcaccttggcccctcttagaagccaattaggccctcagtttctgcagcggggattaatatgattatgaacacccccaatctcccagatgctgattcagccaggagcttaggagggggaggtcactttataagggtctgggggggtcagaacccagagtcatcactagtaacggccgccagtgtgctggaattcgcccttctccaccgccatgttgggctgggtccagagggtgctgcctcagcccccagggacccctcggaagaccaagatgcaggaggaagaggaagtggaaccagagccagagatggaggcggaggtggaaccagaaccgaatcctgaggaggccgagacagagtccgagtccatgccccccgaagagtcattcaaggaggaggaagtggctgtggcagacccaagccctcaggagaccaaggaggctgcccttacttccaccatatccctccgggcccagggcgctgagatttctgaaatgaatagtcccagccacagggtactgacctggctcatgaagggtgtagagaaggtgatcccgcagcctgttcacagcatcacggaggacccggctcagatcctggggcatggcagcactggggacacagggtgcacagatgaacccaatgaggcccttgaggcccaagacactaggcctgggctgcggctgcttctgtggctggagcagaatctggaaagagtgcttcctcagccccccaaatcctctgaggtctggagagatgagcctgcagttgctacaggtgctgcctcagacccagcgcctccaggacgcccccaggaaatggggcccaagctgcaggcccgggagaccccctccctgcccacacccatccccctgcagcccaaggaggaacccaaggaggcaccagctccagagccccagcccggctcccaggcccagacctcctccctgccaccaaccagggaccctgccaggctggtggcatgggtcctgcacaggctggagatggccttgccgcagccagtgctacatgggaaaataggggaacaggagcctgactcccctgggatatgtgatgtgcagaccatcagcatccttcctggaggacaagtggagcctgaccttgtcctagaggaggttgaaccgccctgggaggatgcccaccaggatgtcagtaccagcccacagggtacagaggtggttccagcttatgaagaagagaacaaagctgtggagaagatgcccagagagctgtcccggattgaagaggagaaagaagatgaggaggaggaagaggaagaggaggaggaggaggaagaggaggaggtgactgaggtgctgctggatagctgtgtggtgtcgcaggtgggcgtgggccagagtgaagaagacgggacccggccccagagcacttcagatcagctgtgggaggaagttggggaggaggccaagaaggaggctgaagagaaggccaaggaggaggccgaggaggtggctgaagaggaggctgaaaaggagccccaggactgggcggagaccaaggaggagcctgaggctgaggccgaggctgccagttcaggagtgcctgccacgaaacagcacccagaagtgcaggtggaagatactgatgctgatagctgccccctcatggcagaagagaatccaccctcaaccgtgttgccgccaccgtctcctgccaaatcagacacccttatagtcccaagctcagcctcggggacacacaggaagaagctgccctctgaggatgatgaggctgaagagctcaaggcgttgtcaccagcagagtccccagtggttgcctggtctgaccccaccaccccgaaggacactgatggccaggaccgtgcggcctccacggccagcacaaatagcgccatcatcaacgaccggctccaggagctggtgaagctcttcaaggagcggacagagaaagtgaaggagaaactcattgaccctgacgtcacctctgatgaggagagccccaagccctccccagccaagaaagccccagagccagctccagacacaaagcccgctgaagccgagccagtggaagaggagcactattgcgacatgctctgctgcaagttcaaacaccgcccctggaagaagtaccagtttccccagagcattgacccgctgaccaacctgatgtatgtcctatggctgttcttcgtggtgatggcctggaattggaactgttggctgattcccgtgcgctgggccttcccctaccagaccccggacaacatccaccactggctgctgatggattacctatgcgacctcatctacttcctggacatcaccgtgttccagacacgcctgcagtttgtcagaggcggggacatcattacggacaaaaaggacatgcgaaataattacctgaagtctcgccgcttcaagatggacctgctcagcctcctgcccttggattttctctatttgaaagtcggtgtgaaccccctcctccgcctgccccgctgtttaaagtacatggccttcttcgagtttaacagccgcctggaatccatcctcagcaaagcctacgtgtacagggtcatcaggaccacagcctaccttctctacagcctgcatttgaattcctgtctttattactgggcatcggcctatcagggcctcggctccactcactgggtttacgatggcgtgggaaacagttatattcgctgttactactttgctgtgaagaccctcatcaccatcggggggctgcctgaccccaagacactctttgaaattgtcttccagctgctgaattatttcacgggcgtctttgctttctctgtgatgatcggacagatgagagatgtggtaggggccgccaccgcgggacagacctactaccgcagctgcatggacagcacggtgaagtacatgaatttctacaagatccccaagtccgtgcagaaccgcgtcaagacctggtacgagtacacctggcactcgcaaggcatgctggatgagtcagagctgatggtgcagcttccagacaagatgcggctggacctcgccatcgacgtgaactacaacatcgttagcaaagtcgcactctttcagggctgtgaccggcagatgatctttgacatgctgaagaggcttcgctctgttgtctacctgcccaacgactatgtgtgcaagaagggggagatcggccgtgagatgtacatcatccaggcagggcaagtgcaggtcttgggcggccctgatgggaaatctgtgctggtgacgctgaaagctggatctgtgtttggagaaataagcttgctggctgttgggggcgggaaccggcgcacggccaacgtggtggcgcacgggtttaccaacctcttcatcctggataagaaggacctgaatgagattttggtgcattatcctgagtctcagaagttactccggaagaaagccaggcgcatgctgagaagcaacaataagccccaggaggagaagagcgtgctgatccttccaccccgggcgggcaccccaaagctcttcaacgctgccctcgctatgacaggaaagatgggtggcaagggggcaaaaggcggcaaacttgctcacctccgggcccggctcaaagaactggccgcgctggaggcggctgcaaagcagcaagagttggtggaacaggccaagagctcgcaagacgtcaagggagaggaaggctccgccgccccagaccagcacacgcacccaaaggaggccgccaccgacccacccgcgccccggacgccccccgagcccccggggtctccaccgagctctccaccgcctgcctcccttgggaggccggagggagaggaggaggggccggccgagcccgaagagcactcggtgaggatctgcatgagcccgggcccggagccgggagagcagatcctgtcggtgaagatgccggaggaaagggaggagaaggcggagtaaggtggggtgaggcggatccatggccgcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtctcgagttaagggcgaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 44 - DNA, 4634 bp, synthetic construct
  misc_feature 1..4634: note = 5'ITR-hRHO promoter-CNGB1a-SV40polyA-3'ITR (GenBank)
  source 1..4634: mol_type = other DNA
ggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcagatcgtagccatgctctaggaagatcggaattcgcccttaagcctctcctccctgacctcaggcttcctcctagtgtcaccttggcccctcttagaagccaattaggccctcagtttctgcagcggggattaatatgattatgaacacccccaatctcccagatgctgattcagccaggagcttaggagggggaggtcactttataagggtctgggggggtcagaacccagagtcatcactagtaacggccgccagtgtgctggaattcgcccttctccaccgccatgttgggctgggtccagagggtgctgcctcagcccccagggacccctcggaagaccaagatgcaggaggaagaggaagtggaaccagagccagagatggaggcggaggtggaaccagaaccgaatcctgaggaggccgagacagagtccgagtccatgccccccgaagagtcattcaaggaggaggaagtggctgtggcagacccaagccctcaggagaccaaggaggctgcccttacttccaccatatccctccgggcccagggcgctgagatttctgaaatgaatagtcccagccgcagggtactgacctggctcatgaagggtgtagagaaggtgatcccgcagcctgttcacagcatcacggaggacccggctcagatcctggggcatggcagcactggggacacagggtgcacagatgaacccaatgaggcccttgaggcccaagacactaggcctgggctgcggctgcttctgtggctggagcagaatctggaaagagtgcttcctcagccccccaaatcctctgaggtctggagagatgagcctgcagttgctacaggtgctgcctcagacccagcgcctccaggacgcccccaggaaatggggcccaagctgcaggcccgggagaccccctccctgcccacacccatccccctgcagcccaaggaggaacccaaggaggcaccagctccagagccccagcccggctcccaggcccagacctcctccctgccaccaaccagggaccctgccaggctggtggcatgggtcctgcacaggctggagatggccttgccgcagccagtgctacatgggaaaataggggaacaggagcctgactcccctgggatatgtgatgtgcagaccatcagcatccttcctggaggacaagtggagcctgaccttgtcctagaggaggttgaaccgccctgggaggatgcccaccaggatgtcagtaccagcccacagggtacagaggtggttccagcttatgaagaagagaacaaagctgtggagaagatgcccagagagctgtcccggattgaagaggagaaagaagatgaggaggaggaagaggaagaggaggaggaggaggaagaggaggaggtgactgaggtgctgctggatagctgtgtggtgtcgcaggtgggcgtgggccagagtgaagaagacgggacccggccccagagcacttcagatcagaagctgtgggaggaagttggggaggaggccaagaaggaggctgaagagaaggccaaggaggaggccgaggaggtggctgaagaggaggctgaaaaggagccccaggactgggcggagaccaaggaggagcctgaggctgaggccgaggctgccagttcaggagtgcctgccacgaaacagcacccagaagtgcaggtggaagatactgatgctgatagctgccccctcatggcagaagagaatccaccctcaaccgtgttgccgccaccgtctcctgccaaatcagacacccttatagtcccaagctcagcctcggggacacacaggaagaagctgccctctgaggatgatgaggctgaagagctcaaggcgttgtcaccagcagagtccccagtggttgcctggtctgaccccaccaccccgaaggacactgatggccaggaccgtgcggcctccacggccagcacaaatagcgccatcatcaacgaccggctccaggagctggtgaagctcttcaaggagcggacagagaaagtgaaggagaaactcattgaccctgacgtcacctctgatgaggagagccccaagccctccccagccaagaaagccccagagccagctccagacacaaagcccgctgaagccgagccagtggaagaggagcactattgcgacatgctctgctgcaagttcaaacaccgcccctggaagaagtaccagtttccccagagcattgacccgctgaccaacctgatgtatgtcctatggctgttcttcgtggtgatggcctggaattggaactgttggctgattcccgtgcgctgggccttcccctaccagaccccggacaacatccaccactggctgctgatggattacctatgcgacctcatctacttcctggacatcaccgtgttccagacacgcctgcagtttgtcagaggcggggacatcattacggacaaaaaggacatgcgaaataactacctgaagtctcgccgcttcaagatggacctgctcagcctcctgcccttggattttctctatttgaaagtcggtgtgaaccccctcctccgcctgccccgctgtttaaagtacatggccttcttcgagtttaacagccgcctggaatccatcctcagcaaagcctacgtgtacagggtcatcaggaccacagcctaccttctctacagcctgcatttgaattcctgtctttattactgggcatcggcctatcagggcctcggctccactcactgggtttacgatggcgtgggaaacagttatattcgctgttactactttgctgtgaagaccctcatcaccatcggggggctgcctgaccccaagacactctttgaaattgtcttccagctgctgaattatttcacgggcgtctttgctttctctgtgatgatcggacagatgagagatgtggtaggggccgccaccgcgggacagacctactaccgcagctgcatggacagcacggtgaagtacatgaatttctacaagatccccaagtccgtgcagaaccgcgtcaagacctggtacgagtacacctggcactcgcaaggcatgctggatgagtcagagctgatggtgcagcttccagacaagatgcggctggacctcgccatcgacgtgaactacaacatcgttagcaaagtcgcactctttcagggctgtgaccggcagatgatctttgacatgctgaagaggcttcgctctgttgtctacctgcccaacgactatgtgtgcaagaagggggagatcggccgtgagatgtacatcatccaggcagggcaagtgcaggtcttgggcggccctgatgggaaatctgtgctggtgacgctgaaagctggatctgtgtttggagaaataagcttgctggctgttgggggcgggaaccggcgcacggccaacgtggtggcgcacgggtttaccaacctcttcatcctggataagaaggacctgaatgagattttggtgcattatcctgagtctcagaagttactccggaagaaagccaggcgcatgctgagaagcaacaataagcccaaggaggagaagagcgtgctgatccttccaccccgggcgggcaccccaaagctcttcaacgctgccctcgctatgacaggaaagatgggtggcaagggggcaaaaggcggcaaacttgctcacctccgggcccggctcaaagaactggccgcgctggaggcggctgcaaagcagcaagagttggtggaacaggccaagagctcgcaagacgtcaagggagaggaaggctccgccgccccagaccagcacacgcacccaaaggaggccgccaccgacccacccgcgccccggacgccccccgagcccccggggtctccaccgagctctccaccgcctgcctcccttgggaggccggagggagaggaggaggggccggccgagcccgaagagcactcggtgaggatctgcatgagcccgggcccggagccgggagagcagatcctgtcggtgaagatgccggaggaaagggaggagaaggcggagtaaggtggggtgaggcggatccatggccgcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtctcgagttaagggcgaattcccgataaggatcttcctagagcatggctacgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 45 - protein, 533 aa, Homo sapiens
  REGION 1..533: note = MISC_FEATURE - human RPE65 protein
MSIQVEHPAGGYKKLFETVEELSSPLTAHVTGRIPLWLTGSLLRCGPGLFEVGSEPFYHLFDGQALLHKFDFKEGHVTYHRRFIRTDAYVRAMTEKRIVITEFGTCAFPDPCKNIFSRFFSYFRGVEVTDNALVNVYPVGEDYYACTETNFITKINPETLETIKQVDLCNYVSVNGATAHPHIENDGTVYNIGNCFGKNFSIAYNIVKIPPLQADKEDPISKSEIVVQFPCSDRFKPSYVHSFGLTPNYIVFVETPVKINLFKFLSSWSLWGANYMDCFESNETMGVWLHIADKKRKKYLNNKYRTSPFNLFHHINTYEDNGFLIVDLCCWKGFEFVYNYLYLANLRENWEEVKKNARKAPQPEVRRYVLPLNIDKADTGKNLVTLPNTTATAILCSDETIWLEPEVLFSGPRQAFEFPQINYQKYCGKPYTYAYGLGLNHFVPDRLCKLNVKTKETWVWQEPDSYPSEPIFVSHPDALEEDDGVVLSVVVSPGAGQKPAYLLILNAKDLSEVARAEVEINIPVTFHGLFKKS

(19) United States
(12) Patent Application Publication
(10) Pub. No.: US 2025/0262328 A1
(43) Pub. Date: Aug. 21, 2025

(54) Gene Therapy for the Treatment of CNGB1-linked Retinitis Pigmentosa

(71) Applicants: Stylianos MICHALAKIS, München (DE); Martin BIEL, München (DE)

(72) Inventors: Stylianos MICHALAKIS, München (DE); Martin BIEL, München (DE)

(21) Appl. No.: 19/043,744

(22) Filed: Feb. 3, 2025

Related U.S. Application Data
(63) Continuation of application No. 16/495,826, filed on Sep. 19, 2019, now Pat. No. 12,246,072.
(60) Provisional application No. 62/474,409, filed on Mar. 21, 2017.

(51) Int. Cl. A61K 48/00; A61P 27/02; C07K 14/705; C12N 15/86

(52) U.S. Cl. CPC A61K 48/0058; A61P 27/02; C07K 14/705; C12N 15/86; A01K 2267/0306

(57) ABSTRACT

The present invention relates to a polynucleotide comprising a promoter comprising a human photoreceptor-specific promoter element, a core promoter and at least one transgene. Further, the invention provides a plasmid comprising the polynucleotide, a viral vector comprising the polynucleotide and a pharmaceutical composition comprising the polynucleotide. The invention also relates to the plasmid, the viral vector or the pharmaceutical composition for use as a medicament, in particular for use in the therapy of diseases of the retina.

### CROSS-REFERENCE TO RELATED APPLICATIONS

This application is a continuation of U.S. application Ser. No. 16/495,826, filed on Sep. 19, 2019, which is a US National Phase filing of PCT/IB2018/051905, filed on Mar. 21, 2018, which claims the benefit of U.S. Provisional Application No. 62/474,409, filed on Mar. 21, 2017. The disclosures therein are expressly incorporated entirely by reference.

### SEQUENCE LISTING STATEMENT

A computer readable form of the Sequence Listing is filed with this application by electronic submission and is incorporated into this application by reference in its entirety. The Sequence Listing is contained in the file created on Feb. 3, 2025 having the file name “19-2343-WO-US-CON.xml” and is 125,488 bytes in size.

### BACKGROUND OF THE INVENTION

Retinitis pigmentosa (RP) is a term that is used to refer to a genetically diverse group of inherited degenerative diseases of the retina affecting the photoreceptors. The genetic mutation concerns genes that are either exclusively or primarily expressed in rod photoreceptors. Accordingly, the disease is characterized by a primary impairment or loss of rod function and structure. Deterioration of rods is followed by a secondary degeneration of the cones. Onset and time course of retinal degeneration varies from early-onset and fast progressing forms to late-onset and slow progressing forms, respectively. The most common symptoms of RP are night blindness, progressive constriction of the visual field, and abnormal accumulation of pigmentation in the retina. Clinical features include characteristically shaped pigmentary deposits and a progressive attenuation of retinal vessels. In many cases RP finally leads to legal blindness. The overall prevalence of RP is estimated to 1:4,000. RP is genetically very heterogeneous and the number of identified RP genes approximates 50 (Daiger S P, et al. (1998) Investigative Ophthalmology and Visual Science (Supplement) 39:S295). Many disease genes encode proteins required for light detection and processing (e.g. rhodopsin) or for maintenance of rod cellular morphology (e.g. peripherin-2). 10-25% of RP cases show an autosomal dominant pattern of inheritance (adRP), 6-18% are X-linked (xRP) and 20-30% are autosomal recessively inherited (arRP). Another 40-50% are sporadic arRP and is genetically the most diverse RP subgroup and none of the known disease genes has a relative frequency of more than 15%. The most prevalent arRP genes are EYS (5-12%), USH2A (5-15%), CRB1 (approx. 5%), and PDE6B (4-10%). However, most likely due to founder effects these values vary between different subpopulations and across regions.

CNGB1 encodes the beta subunit of the rod cyclic nucleotide-gated (CNG) channel (RP45 locus). Mutations in the RP45 locus causing so-called CNGB1-linked RP or RP type 45, respectively, are found in 2-4% of arRP cases (Hartong D T, et al. (2006) Lancet 368 (9549): 1795-1809). Therefore, the estimated number of patients with CNGB1-linked arRP is approximately 900 in Germany and 5,000 in the EU. Vision impairment is considered one of the most important non-mortal handicaps with high clinical and socioeconomic importance. RP Patients suffer from severe loss of quality of life throughout an extensive period of their lifetime. Unfortunately, no curative or symptomatic treatments of RP exist. Clinical experts and health organizations list RP as one of the top candidates for gene therapy. Previously, it could be demonstrated that gene supplementation therapy restores vision and delays degeneration in the CNGB1 (−/−) mouse model of retinitis pigmentosa (Koch S, et al. (2012) Hum Mol. Genet. 21 (20): 4486-96) by using recombinant AAV2/8 vector comprising the mouse Cngb1 gene under the control of the mouse rhodopsin (Rho) promoter: AAV2/8 (Y733F)-Rho-Cngb1. The vector was injected into the eye of mice with a genetic deletion in exon 26 of the gene encoding Cngb1 (Cngb1 KO). The injection enhanced survival of photoreceptors and improved retinal function. However, several issues render this approach less promising for the treatment of humans suffering from retinal degenerations due to CNGB1-linked RP:
(a) the rAAV cis vector genome size (5.0 kb) was above the size of the wildtype AAV genome (<4.7 kb);
(b) a murine rhodopsin (Rho) gene promoter was used; and
(c) a murine Cngb1 gene sequence was used.

Petersen-Jones et al. (2016) Invest. Ophthalmol. 57:1842, describe an rAAV2/5 vector comprising the coding sequence of the canine Cngb1 gene (cCngb1) under control of a human rhodopsin kinase 1 (hGRK1) promoter: AAV5-hGRK1-cCngb1. The vector was injected into the eye of dogs with a mutation in exon 26 of the Cngb1 gene. The injection improved retinal function. However, the following issues render this approach less promising for the treatment of humans suffering from retinal degenerations due to CNGB1-linked RP:
(a) the hGRK1 promoter used in this approach drives expression in rods, but also off-target expression in cone photoreceptors. This off-target expression could have a negative impact on retinal function and morphology; and
(b) a canine Cngb1 gene sequence was used.

Thus, there is a need in the art to identify transgenic elements that have a small size without negatively affecting or losing their activity in the in vivo situation.

### SUMMARY OF THE INVENTION

The present invention is based on the surprising discovery that a short part of the human rod promoter transfers rod photoreceptor-specific expression to transgenes operably linked to this promoter element in vivo. When the promoter element defined herein was used in an in vivo setting, stable expression of a transgene was observed. The expression level was suitable to improve the visual capabilities of the test animals infected with an adeno-associated virus vector comprising the transgene. This surprising finding provides inter alia the following advantages over the prior art: (i) reduction of the size of the construct that is introduced into a cell, (ii) an increase of the packaging efficiency of the transgene into viral vectors, (iii) a decrease of the chance that recombination events occur in vivo, (iv) an increase the efficiency of introduction of the transgene into the target cells, in particular into the nucleus of the target cell; (v) a suitable expression level in a human patient to treat rod associated diseases, (vi) preservation and/or improvement of retinal function and (vii) preservation and/or improvement of vision.

In a first aspect the invention relates to a polynucleotide comprising in this order:
a) a promoter comprising a human rod photoreceptor-specific promoter element (hRPSPE) comprising, consisting essentially of or consisting of the nucleic acid sequence according to SEQ ID NO: 1 or variants thereof and a core promoter (CP); and
b) a transgene (TG) operably linked to the promoter of a);
wherein the variant of SEQ ID NO: 1 comprises one or more nucleic acid substitutions outside nucleotide positions 6 to 13, 32 to 40, 70 to 83, and 87 to 94 of SEQ ID NO: 1 and wherein the length of the promoter is in particular 350 bases or less.

In certain exemplary embodiments, the 5′ end of the hRPSPE is at a nucleic acid position from 1 to 160 and the 3′ end at a nucleic acid position from 290 to 310 of SEQ ID NO: 2 or variants thereof.

In certain exemplary embodiments, the CP comprises a TATA-box and/or an initiator (Inr).

In certain exemplary embodiments, the 5′ end of the promoter is at a nucleic acid position from 1 to 160 and the 3′ end at a nucleic acid position from 340 to 350 of SEQ ID NO: 2 or variants thereof.

In certain exemplary embodiments, the transgene comprises a nucleic acid encoding a protein that maintains or improves the physiological function of rods.

In certain exemplary embodiments, the transgene: (i) comprises a nucleic acid encoding the human rod cyclic nucleotide-gated channel beta subunit (hCNGB1), ABCA4, AIPL1, BEST1, CACNA1F, CLN3, CLRN1, CNGA1, CEP290, CRB1, CRB2, CRX, GPR98, GUCA1A, GUCA1B, MY07A, NRL, PDE6A, PDE6B, PRPH2, PROM1, RHO, ROM1, RP1, RP2, RPE65, RPGR, SAG, USHIC, USHIG, USH2A or functional fragments or variants thereof; a nucleic acid encoding a miRNA or shRNA targeting a mRNA encoding a dominant negative mutant thereof; and/or a nucleic acid encoding an antibody or antibody binding fragment that specifically binds to a dominant negative mutant thereof; or (ii) comprises a nucleic acid encoding a protein that inhibits proliferation of rod cells, preferably a toxin; a prodrug converting enzyme, e.g. thymidine kinase; cell cycle inhibitors, e.g. retinoblastoma protein (pRB), p53, p21CIP1, p27KIP1 and p57KIP2; comprises a mRNA encoding a dominant negative mutant of the cell cycle inhibitor thereof; and/or comprises a nucleic acid encoding a dominant negative mutant of a cell cycle inhibitor thereof.

In certain exemplary embodiments, the hCNGB1 comprises an amino acid sequence according to SEQ ID NOs: 3, 40, or 41, or variants thereof.

In certain exemplary embodiments, the polynucleotide comprises one or more further nucleotide sequence elements selected from the group consisting of: (i) a polyadenylation signal (PAS); and/or (ii) one or two inverted terminal repeat (ITR) sequences; and/or (iii) viral nucleotide sequences necessary to form an infectious viral vector, preferably an adenovirus, a retrovirus, a lentivirus, a vaccinia/poxvirus, or a herpesvirus vector, in particular herpes simplex virus (HSV) vector.

In certain exemplary embodiments, the polyadenylation signal comprises, essentially consists or consists of a Simian-Virus 40 PAS.

In certain exemplary embodiments, the polyadenylation signal comprises, essentially consists or consists of a nucleic acid according to SEQ ID NO: 4 or functional variants thereof.

In certain exemplary embodiments, the ITR sequence is an adeno-associated virus (AAV) ITR.

In certain exemplary embodiments, the AAV is AVV serotype 2, 5, 8 or 9.

In certain exemplary embodiments, the promoter and the transgene are flanked at their 5′ with a L-ITR and at their 3′ end with a R-ITR.

In certain exemplary embodiments, the L-ITR comprises, essentially consists or consists of a sequence according to SEQ ID NO: 5 or variants thereof and/or the R-ITR comprises, essentially consists or consists of a sequence according to SEQ ID NO: 6 or variants thereof.

In certain exemplary embodiments, the total length of the polynucleotide is 5200 bases or less, preferably 5100 bases or less, more preferably 5000 bases or less.

In a second aspect the invention further relates to a plasmid comprising the polynucleotide of the first aspect.

In certain exemplary embodiments, the plasmid comprises a nucleic acid sequence according to SEQ ID NOs: 7, 42-44, or variants thereof.

A third aspect of the invention relates to a viral vector comprising the polynucleotide of the first aspect of the invention.

In certain exemplary embodiments, the virus is selected from the group consisting of AAV2, AAV5, AAV8, AVV9 or variants thereof.

A fourth aspect of the invention relates to the polynucleotide according to the first aspect of the invention, the plasmid of the second aspect of the invention and/or the viral vector according to the third aspect of the invention for use as a medicament.

A fifth aspect of the invention relates to a pharmaceutical composition comprising the polynucleotide according to the first aspect of the invention, the plasmid of the second aspect of the invention and/or the viral vector according to the third aspect of the invention, and a pharmaceutically acceptable carrier.

A sixth aspect of the invention relates to the polynucleotide according to the first aspect of the invention, the plasmid according to the second aspect of the invention and/or the viral vector according to the third aspect of the invention for use in the therapy of a disease of the retina, in particular retinal degeneration.

In certain exemplary embodiments, the route of administration is selected from intraocular, intrabulbar, intravitreal or subretinal.

In certain exemplary embodiments, the retinal degeneration is associated with a genetic mutation, substitution, and/or deletion.

In certain exemplary embodiments, the retinal degeneration is selected from the group consisting of night blindness, blindness, retinal degeneration, retinal dystrophy and retinitis pigmentosa.

In certain exemplary embodiments, the retinitis pigmentosa is CNGB1-linked retinitis pigmentosa or retinitis pigmentosa type 45 (RP45).

A seventh aspect of the invention relates to a polynucleotide comprising in this order:
a) a human rhodopsin promoter comprising the nucleic acid sequence according to SEQ ID NO: 9 or variants thereof; and
b) at least one transgene (TG) operably linked to the promoter of a).

In certain exemplary embodiments, the transgene comprises a nucleic acid encoding a protein that maintains or improves a physiological function of rods.

In certain exemplary embodiments, the transgene: (i) comprises a nucleic acid encoding the human rod cyclic nucleotide-gated channel beta subunit (hCNGB1), ABCA4, AIPL1, BEST1, CACNA1F, CLN3, CLRN1, CNGA1, CEP290, CRB1, CRB2, CRX, GPR98, GUCA1A, GUCA1B, MYO7A, NRL, PDE6A, PDE6B, PRPH2, PROM1, RHO, ROM1, RP1, RP2, RPE65, RPGR, SAG, USH1C, USH1G, USH2A or functional fragments or variants thereof; a nucleic acid encoding a miRNA or shRNA targeting a mRNA encoding a dominant negative mutant thereof; and/or a nucleic acid encoding an antibody or antibody binding fragment that specifically binds to a dominant negative mutant thereof; or (ii) comprises a nucleic acid encoding a protein that inhibits proliferation of rod cells, preferably a toxin; a prodrug converting enzyme, e.g. thymidine kinase; cell cycle inhibitors, e.g. retinoblastoma protein (pRB), p53, p21CIP1, p27KIP1 and p57KIP2; comprises a mRNA encoding a dominant negative mutant of the cell cycle inhibitor thereof; and/or comprises a nucleic acid encoding a dominant negative mutant of a cell cycle inhibitor thereof.

In certain exemplary embodiments, the polynucleotide comprises one or more further nucleotide sequence elements selected from the group consisting of:
(i) a polyadenylation signal (PAS);
(ii) one or two inverted terminal repeat (ITR) sequences; and
(iii) viral nucleotide sequences necessary to form an infectious viral vector, preferably an adenovirus, a retrovirus, a lentivirus, a vaccinia/poxvirus, or a herpesvirus vector, in particular herpes simplex virus (HSV) vector.

In certain exemplary embodiments, the polyadenylation signal comprises a Simian-Virus 40 PAS.

In certain exemplary embodiments, the ITR sequence is an adeno-associated virus (AAV) ITR.

In certain exemplary embodiments, the AAV is AVV serotype 2, 5, 8 or 9.

An eighth aspect of the invention relates to a viral vector comprising the polynucleotide according to the seventh aspect of the invention.

In certain exemplary embodiments, the virus is selected from the group consisting of AAV2, AAV5, AAV8, AVV9 or variants thereof.

A ninth aspect of the invention relates to a method for treating retinal degeneration in a subject in need thereof, comprising administering to the subject a therapeutically effective amount of a polynucleotide according to the seventh aspect of the invention, or the viral vector according to the eighth aspect of the invention.

In certain exemplary embodiments, the polynucleotide or viral vector comprises the nucleic acid sequence set forth in SEQ ID NO: 43.

A tenth aspect of the invention relates to a method for treating retinitis pigmentosa in a subject in need thereof, comprising administering to the subject a therapeutically effective amount of a polynucleotide according to the seventh aspect of the invention, or the viral vector according to the eighth aspect of the invention.

In certain exemplary embodiments, the polynucleotide or viral vector comprises the nucleic acid sequence set forth in SEQ ID NO: 43.

An eleventh aspect of the invention relates to a method for treating retinal degeneration in a subject in need thereof, wherein the retinal degeneration is characterized by a defect or absence of CNGB1 in the retinal cells of the subject, the method comprising administering to the subject a therapeutically effective amount of a viral vector comprising the nucleic acid sequence set forth in SEQ ID NO: 43.

In certain exemplary embodiments, the retinal degeneration is CNGB1-linked retinitis pigmentosa or retinitis pigmentosa type 45 (RP45).

A twelfth aspect of the invention relates to a method for treating CNGB1-linked retinitis pigmentosa or retinitis pigmentosa type 45 (RP45) in a subject in need thereof, comprising subretinal administration to the subject a therapeutically effective amount of a viral vector comprising the nucleic acid sequence set forth in SEQ ID NO: 43.

A thirteenth aspect of the invention relates to a polynucleotide comprising in this order:
a) a promoter comprising a human rod photoreceptor-specific promoter element (hRPSPE) comprising the nucleic acid sequence according to SEQ ID NO: 1 or variants thereof and a core promoter (CP); and
b) a transgene encoding the human rod cyclic nucleotide-gated channel beta subunit (hCNGB1) operably linked to the promoter of a), wherein the variant of SEQ ID NO: 1 comprises one or more nucleic acid substitutions outside nucleotide positions 6 to 13, 32 to 40, 70 to 83, and 87 to 94 of SEQ ID NO: 1.

A fourteenth aspect of the invention relates to a pharmaceutical composition comprising a polynucleotide comprising in this order:
a) a promoter comprising a human rod photoreceptor-specific promoter element (hRPSPE) comprising the nucleic acid sequence according to SEQ ID NO: 1 or variants thereof and a core promoter (CP); and
b) a transgene encoding the human rod cyclic nucleotide-gated channel beta subunit (hCNGB1) operably linked to the promoter of a);
wherein the variant of SEQ ID NO: 1 comprises one or more nucleic acid substitutions outside nucleotide positions 6 to 13, 32 to 40, 70 to 83, and 87 to 94 of SEQ ID NO: 1, and
a pharmaceutically acceptable carrier.

A fifteenth aspect of the invention relates to a pharmaceutical composition comprising a viral vector comprising the nucleic acid sequence set forth in SEQ ID NO: 43 and a pharmaceutically acceptable carrier.

### LIST OF FIGURES

In the following, the content of the figures comprised in this specification is described. In this context please also refer to the detailed description of the invention above and/or below.

FIG. 1 shows the structure of the rAAV hRHO194.hCNGB1 vector genome.

FIG. 2 shows the pGL2.0-hRHO194-hCNGB1a-SV40 cis vector plasmid map.

FIGS. 3A-3B depict representative confocal images showing native eGFP fluorescence in wild type mice treated with a version of the vector expressing eGFP instead of hCNGB1. These representative confocal images show native eGFP fluorescence in retinal cross-sections from 8-week-old wildtype mice treated subretinally at 4 weeks with rAAV.hRHO194.eGFP vector. Intense and rod-specific eGFP signal was observed in treated animals (FIG. 3A), but was absent in non-injected controls (FIG. 3B).

FIGS. 4A-4B show representative ERG measurements from CNGB1 (−/−) mice treated with the vector according to the invention; Electroretinography (ERG) measurement data from CNGB1 (−/−) mice treated in one eye with the vector according to the invention. (FIG. 4A) Representative ERG traces obtained upon 4.4 cd/m² single flash stimulation. The hatched trace is from the treated eye and the black trace from the untreated eye of a CNGB1 (−/−) mouse at 4 months after treatment. (FIG. 4B) Summary graph showing the ERG b-wave amplitudes measured under the same conditions from wild type mice (grey), treated CNGB1 (−/−) mice (dark grey) and untreated CNGB1 (−/−) mice (black). * p<0.05, Student's t-test, N=4.

FIGS. 5A-5C show optical coherence tomography (OCT) measurements of photoreceptor layer thickness from CNGB1 (−/−) mice treated in one eye with the vector according to the invention. (FIGS. 5A-5B) Representative OCT scans from treated (FIG. 5A) and untreated eye (FIG. 5B). The thickness of the photoreceptor layer is marked with a vertical black bar. Quantification of photoreceptor layer thickness using OCT (FIG. 5C). ***p<0.001, 1 way ANOVA, N=9.

FIGS. 6A-6B depict representative confocal images from immunohistological stainings of hCNGB1 in CNGB1 (−/−) mice treated with the vector according to the invention (FIG. 6A) or untreated (FIG. 6B).

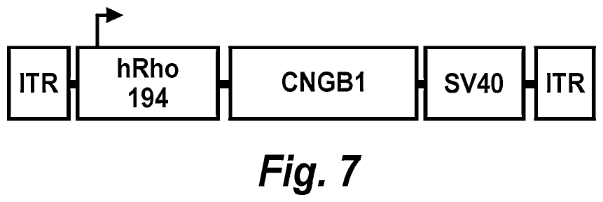

FIG. 7 depicts a schematic showing the general vector design according to the invention.

FIGS. 8A-8B show representative ERG measurements from CNGB1 (−/−) mice treated with the vector according to the invention (FIG. 8A). Representative ERG measurements in wild-type and CNGB1 (−/−) mice before treatment (FIG. 8B).

FIGS. 9A-9B depict representative confocal images from immunohistological stainings of hCNGB1 in CNGB1 (−/−) mice treated with the vector according to the invention (FIG. 9A), and untreated mice (FIG. 9B).

FIGS. 10A-10C depict OCT analysis revealing a significant delay in retinal degeneration. General injection schedule of the vector according to the invention (FIG. 10A). OCT images collected at 9 months in CNGB1 (−/−) mice treated with the vector according to the invention (FIG. 10B), and untreated mice (FIG. 10C).

FIGS. 11A-11E depict restoration of rod function by two months in CNGB1 (−/−) mice treated with the vector according to the invention. Representative ERG B-wave measurements in CNGB1 (−/−) mice treated with the vector according to the invention, and untreated mice (FIG. 11A). Summary graph showing the ERG b-wave amplitudes measured in response to a light stimulus of −0.5 log (cd s/m2) in CNGB1 (−/−) mice treated with the vector according to the invention, and untreated mice (FIG. 11B). OCT measurements of photoreceptor layer thickness from CNGB1 (−/−) mice treated with the vector according to the invention (FIG. 11C), and untreated mice (FIG. 11D). Quantification of photoreceptor layer thickness using OCT (FIG. 11E). N=6.

FIGS. 12A-12B depict obvious ERG rescue observed in eyes of CNGB1 (−/−) dogs treated with the vector according to the invention, and untreated dogs, using a rod-specific stimulus (FIG. 12A), and a flicker response (FIG. 12B).

FIGS. 13A-13B depict vision testing data showing that CNGB1 (−/−) dogs treated with the vector according to the invention have rod-mediated vision and improved vision testing performance. Restored rod vision indicated by improved performance in correct exit choice (FIG. 13A), and time to exit (FIG. 13B).

FIG. 14A-14B depict ERG measurements showing improvement in A- and B-wave amplitude in CNGB1 (−/−) dogs treated with the vector according to the invention. A-wave amplitude indicated-improvement in response threshold in treated eyes was found to be greater than 1.5 log units (FIG. 14A). B-wave amplitude-indicated improvement in response threshold in treated eyes was found to be greater than 2 log units (FIG. 14B).

List of Sequences

| | |
|---|---|
| SEQ ID NO: 1 | Sequence of a 99 nucleotides long fragment of the human rhodopsin |
| | promoter comprising the core tissue specific elements; |
| SEQ ID NO: 2 | Sequence of a 350 nucleotides long fragment of the human rhodopsin |
| | promoter comprising the tissue specific elements and the transcriptional |
| | start site; |
| SEQ ID NO: 3 | Sequence of the human CNGB1 protein; |
| SEQ ID NO: 4 | Sequence of a polyadenylation signal SV40; |
| SEQ ID NO: 5 | Sequence of the left inverted terminal repeat (L-ITR); |
| SEQ ID NO: 6 | Sequence of the right inverted terminal repeat (R-ITR); |
| SEQ ID NO: 7 | Sequence of vector construct: pGL2.0-hRho194-hCNGB1a-SV40; |
| SEQ ID NO: 8 | Sequence of the human CNGB1 gene; |
| SEQ ID NO: 9 | Sequence of a fragment of the human rhodopsin promoter 194 bp; |
| SEQ ID NO: 10 | Sequence of the human Abca4 protein; |
| SEQ ID NO: 11 | Sequence of the human AIPL1 protein; |
| SEQ ID NO: 12 | Sequence of the human BEST1 protein; |
| SEQ ID NO: 13 | Sequence of the human CACNA1F protein; |
| SEQ ID NO: 14 | Sequence of the human CLN3 protein; |
| SEQ ID NO: 15 | Sequence of the human CLRN1 protein; |
| SEQ ID NO: 16 | Sequence of the human CNGA1 protein; |
| SEQ ID NO: 17 | Sequence of the human CEP290 protein; |
| SEQ ID NO: 18 | Sequence of the human CRB1 protein; |
| SEQ ID NO: 19 | Sequence of the human CRB2 protein; |
| SEQ ID NO: 20 | Sequence of the human CRX protein; |
| SEQ ID NO: 21 | Sequence of the human GPR98 protein; |
| SEQ ID NO: 22 | Sequence of the human GUCA1A protein; |
| SEQ ID NO: 23 | Sequence of the human GUCA1B protein; |
| SEQ ID NO: 24 | Sequence of the human MYO7A protein; |
| SEQ ID NO: 25 | Sequence of the human NRL protein; |
| SEQ ID NO: 26 | Sequence of the human PDE6A protein; |
| SEQ ID NO: 27 | Sequence of the human PDE6B protein; |
| SEQ ID NO: 28 | Sequence of the human PRPH2 protein; |
| SEQ ID NO: 29 | Sequence of the human PROM1 protein; |
| SEQ ID NO: 30 | Sequence of the human RHO protein; |
| SEQ ID NO: 31 | Sequence of the human ROM1 protein; |
| SEQ ID NO: 32 | Sequence of the human RP1 protein; |
| SEQ ID NO: 33 | Sequence of the human RP2 protein; |
| SEQ ID NO: 34 | Sequence of the human RPGR protein; |
| SEQ ID NO: 35 | Sequence of the human SAG protein; |
| SEQ ID NO: 36 | Sequence of the human USH1C protein; |
| SEQ ID NO: 37 | Sequence of the human USH1G protein; |
| SEQ ID NO: 38 | Sequence of the human USH2A protein; |
| SEQ ID NO: 39 | Sequence of the human NR2E3 protein; |
| SEQ ID NO: 40 | Sequence of the human CNGB1 protein (next generation sequencing; |
| | NGS); |
| SEQ ID NO: 41 | Sequence of the human CNGB1 protein (GenBank NG_016351); |
| SEQ ID NO: 42 | Sequence of 5′ITR-hRHO promoter-CNGB1a-SV40poly A-3′ITR; |
| SEQ ID NO: 43 | Sequence of 5′ITR-hRHO promoter-CNGB1a-SV40poly A-3′ITR (NGS); |
| SEQ ID NO: 44 | Sequence of 5′ITR-hRHO promoter-CNGB1a-SV40poly A-3′ITR |
| | (GenBank); and |
| SEQ ID NO: 45 | Sequence of the human RPE65 protein. |

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Several documents are cited throughout the text of this specification. Each of the documents cited herein (including all patents, patent applications, scientific publications, manufacturer's specifications, instructions etc.), whether supra or infra, is hereby incorporated by reference in its entirety. Nothing herein is to be construed as an admission that the invention is not entitled to antedate such disclosure by virtue of prior invention. Some of the documents cited herein are characterized as being “incorporated by reference”. In the event of a conflict between the definitions or teachings of such incorporated references and definitions or teachings recited in the present specification, the text of the present specification takes precedence.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Definitions

To practice the present invention, unless otherwise indicated, conventional methods of chemistry, biochemistry, and recombinant DNA techniques are employed which are explained in the literature in the field (cf., e.g., Molecular Cloning: A Laboratory Manual, 2^{nd} Edition, J. Sambrook et al. eds., Cold Spring Harbor Laboratory Press, Cold Spring Harbor 1989).

In the following, some definitions of terms frequently used in this specification are provided. These terms will, in each instance of its use, in the remainder of the specification have the respectively defined meaning and preferred meanings.

As used in this specification and the appended claims, the singular forms “a”, “an”, and “the” include plural referents, unless the content clearly dictates otherwise.

The term “nucleic acid” as used in this specification comprises polymeric or oligomeric macromolecules, or large biological molecules, essential for all known forms of life. Nucleic acids, which include DNA (deoxyribonucleic acid) and RNA (ribonucleic acid), are made from monomers known as nucleotides. Most naturally occurring DNA molecules consist of two complementary biopolymer strands coiled around each other to form a double helix. The DNA strand is also known as polynucleotides consisting of nucleotides. Each nucleotide is composed of a nitrogen-containing nucleobase as well as a monosaccharide sugar called deoxyribose or ribose and a phosphate group. Naturally occurring nucleobases comprise guanine (G), adenine (A), thymine (T), uracil (U) or cytosine (C). The nucleotides are joined to one another in a chain by covalent bonds between the sugar of one nucleotide and the phosphate of the next, resulting in an alternating sugar-phosphate backbone. If the sugar is deoxyribose, the polymer is DNA. If the sugar is ribose, the polymer is RNA. Typically, a polynucleotide is formed through phosphodiester bonds between the individual nucleotide monomers. In the context of the present invention the term “nucleic acid” includes but is not limited to ribonucleic acid (RNA), deoxyribonucleic acid (DNA), and mixtures thereof such as e.g. RNA-DNA hybrids (within one strand), as well as cDNA, genomic DNA, recombinant DNA, CRNA and mRNA. A nucleic acid may consist of an entire gene, or a portion thereof, the nucleic acid may also be a miRNA, siRNA, piRNA or shRNA. miRNAs are short ribonucleic acid (RNA) molecules, which are on average 22 nucleotides long but may be longer and which are found in all eukaryotic cells, i.e. in plants, animals, and some viruses, which functions in transcriptional and post-transcriptional regulation of gene expression. miRNAs are post-transcriptional regulators that bind to complementary sequences on target messenger RNA transcripts (mRNAs), usually resulting in translational repression and gene silencing. Small interfering RNAs (siRNAs), sometimes known as short interfering RNA or silencing RNA, are short ribonucleic acid (RNA molecules), between 20-25 nucleotides in length. They are involved in the RNA interference (RNAi) pathway, where they interfere with the expression of specific genes. A short hairpin RNA (shRNA) also referred to as small hairpin RNA is an artificial RNA molecule with a tight hairpin turn that can be used to silence target gene expression via RNA interference (RNAi). Expression of shRNA in cells is typically accomplished by delivery of plasmids or through viral vectors.

The term “polynucleotide” when used in the context of the present invention, refers to a nucleic acid not restricted to a specific number of nucleotides in length.

The term “human rod photoreceptor” used in the context of the present invention refers to a special type of cells, i.e. photoreceptor cells. The retina of the human eye contains two type of photoreceptor: rods and cones. On average, there are approximately 90 million rod cells in the human retina. Rods are more sensitive than cones. However, they are not sensitive to color. They are responsible for dark-adapted, or scotopic, vision. Rods are usually found concentrated at the outer edges of the retina and are used in peripheral vision. Thus, the peripheral vision is more light-sensitive, enabling one to see dimmer objects in your peripheral vision. Rod cells are more sensitive than cone cells and are almost entirely responsible for night vision. Rods employ a sensitive photopigment called rhodopsin. Photoreceptors are highly specialized, light-sensitive neurons and designed for capturing light quanta triggering a change in the cell's membrane potential. Rod photoreceptors enable dim light vision, whereas cone photoreceptors mediate color vision and high visual acuity under brighter light conditions. Only one type of rod photoreceptor, carrying the rhodopsin visual pigment, is present in the vertebrate retina, including in mouse and human. When in its ‘ready to be activated’ state, each opsin molecule is covalently bound to a light-sensitive chromophore, 11-cis retinal. Upon photon capture, the chromophore isomerizes to all-trans retinal, causing a conformational change in rhodopsin and activation to meta-rhodopsin II. This initiates the process of phototransduction, a cascade of biochemical events that culminate in closure of ionic channels in the cell membrane hyperpolarization of the photoreceptor and transmission of the signal(s) to second-order neurons in the inner retina via modulation of neurotransmitter release at the synaptic terminals. The integrity and function of photoreceptors are absolutely crucial for vision, and mutations that affect photoreceptor function or survival disrupt the phototransduction process, leading to vision loss.

The term “promoter” in the context of the present invention refers to a nucleotide sequence that comprises both elements required for transcription control including binding sites for transcriptional activator and repressor proteins and elements that initiate transcription. The binding sites for transcriptional activator and/or repressor proteins are typically located directly upstream or at the 5′ end of the transcription initiation site comprised within the core promoter. Thus, RNA polymerase and the necessary transcription factors bind to the promoter sequence and initiate transcription. Promoter sequences define the direction of transcription and indicate which DNA strand will be transcribed; this strand is known as the sense strand. The promoter of the present invention transfers rod-photoreceptor specificity on a transgene that is positioned downstream, i.e. at the 3′ end of the promoter.

The term “core promoter” (CP) is used herein in its ordinary sense to refer to a nucleotide region including a DNA regulatory sequence, wherein the regulatory sequence is derived from a gene which is capable of binding RNA polymerase and initiating transcription of a downstream (3′-direction) coding sequence. Thus, the core promoter is the minimal portion of the promoter required to properly initiate gene transcription and contains a binding site for RNA polymerase (RNA polymerase I, RNA polymerase II, or RNA polymerase III). The RNA polymerase binding site of the CP is approximately 25 to 35 bases upstream (5′) from the transcriptional TSS. The core promoter may comprise a so-called TATA box (also called the Goldberg-Hogness box) which is a DNA sequence (cis-regulatory element) often found in the promoter region of genes in archaea and eukaryotes. The TATA box has the core DNA sequence 5′-TATAAA-3′ or variants thereof, which is usually followed by three or more adenine bases. The TATA box is usually located 25-35 base pairs upstream of the transcription start site. The core promoter may also be TATA box-less. Genes lacking a TATA box use an initiator element or downstream core promoter instead. The core promoter also may comprise an initiator (Inr). An Inr consists of an initiator motif and is similar in function to the TATA box. The Inr element facilitates binding to transcription factor II D (TFIID).

The term “human rod photoreceptor specific promoter element” (hRPSPE) as used in the context of the present invention means a promoter element which mediates transcription of the downstream transgene only in rod cells, in particular in human rod cells. Use of the tissue-specific promoter allows a protein or a functional RNA to be expressed tissue-specifically in retina cells of the human eye. The hRPSPE only comprises parts or fragments of the naturally occurring human rod photoreceptor promoter sequence.

The term “gene” or “coding sequence” or a sequence which “encodes” a particular protein or peptide is used in the context of the invention to refer to a nucleic acid molecule that is transcribed (in the case of DNA) and translated (in the case of mRNA) into a polypeptide in vitro or in vivo when placed under the control of appropriate regulatory sequences. The boundaries of the gene are determined by a start codon at the 5′ (i.e., amino) terminus and a translation stop codon at the 3′ (i.e., carboxy) terminus. The term gene includes, but is not limited to prokaryotic or eukaryotic mRNA, cDNA, genomic DNA sequences from prokaryotic or eukaryotic DNA, and even synthetic DNA sequences. A transcription termination sequence will usually be located 3′ to the gene sequence.

The term “transgene” is used in the context of the present invention to refer to a gene that is removed from its natural context and placed under the expression control of a heterologous promoter. An example of a transgene of the present invention is the “rod cyclic nucleotide-gated channel beta” (CNGB1) gene which encodes the rod cyclic nucleotide-gated channel beta subunit. In further embodiments transgenes may comprise the human proteins: ATP Binding Casette Subfamily A Member 4 (ABCA4), Aryl Hydrocarbon Receptor Interacting Protein Like 1 (AIPL1), Bestrophin 1 (BEST1), Calcium Voltage-Gated Channel Subunit Alpha 1 F (CACNA1F), Ceroid-Lipofuscinosis Neuronal 3 (CLN3), Clarin 1 (CLRN1), Cyclic Nucleotide Gated Channel Alpha 1 (CNGA1), Centrosomal Protein 290 (CEP290), Crumbs 1 (CRB1), Crumbs 2 (CRB2), Cone-Rod Homeobox (CRX), G-Protein Coupled Receptor 98 (GPR98), Guanylate Cyclase Activator 1A (GUCA1A), Guanylate Cyclase Activator 1B (GUCA1B), Myosin VIIA (MYO7A), Nuclear Receptor Subfamily 2 Group E Member 3 (NR2E3), Neural Retina Leucine Zipper (NRL), Phosphodiesterase 6A (PDE6A), Phosphodiesterase 6B (PDE6B), Peripherin 2 (PRPH2), Prominin 1 (PROM1), Rhodopsin (RHO), Retinal Outer Segment Membrane Protein 1 (ROM1), Retinitis Pigmentosa 1 Protein (RP1), Retinitis Pigmentosa 2 Protein (RP2), Retinal Pigment Epithelium Specific Protein 65 (RPE65), Retinitis Pigmentosa GTPase Regulator (RPGR), S-Antigen Visual Arrestin (SAG), Usher Syndrome Type-1C Protein (USHIC), Usher Syndrome Type-1G Protein (USH1G), Usher Syndrome Type-2A Protein (USH2A) or functional fragments or variants thereof. The amino acid sequences of particular embodiments of above proteins are indicated in SEQ ID NO: 10 to 41, and 45. Functional fragments are those fragments that maintain the function of the respective protein in normal function of the rod photoreceptor. Similarly, variants also maintain the function of the respective protein in the rod photoreceptor. Proteins with long amino acid sequences, for example human CACNA1F, CEP290, GPR98, MYO7A, RP1 and USH2A protein, which are too long to be encoded by a transgene deliverable by the respectively chosen vector system, in particular AAV vector. To fit the size limitation of AAV vectors “split vector” technologies using the development of an intein-mediated split system for gene therapy can be used. By the use of split-inteins the packaging limit of the AAV can be bypassed. Therefore, each half transgene of interest can be fused to the corresponding split-intein moiety and, only upon co-expression, the intein-mediated trans-splicing occurs and the full transgenic protein is reconstituted. Thus, it would be possible to construct two vectors encoding fragments of the transgenic protein that would upon co-transduction assemble in the target cell into the full-length functional protein.

The term “CNGB1” as used in the context of the present application refers to either the gene or the protein encoded by the CNGB1 gene, i.e. the rod photoreceptor cGMP-gated cation channel which helps regulate ion flow into the rod photoreceptor outer segment in response to light-induced alteration of the levels of intracellular cGMP. This channel consists of two subunits, alpha and beta, with the protein encoded by this gene representing the beta subunit. Diseases associated with CNGB1 and defects in this gene include Retinitis Pigmentosa 45 and CNGB1-related Retinitis Pigmentosa. The CNGB1 subunit of cyclic nucleotide-gated channels plays an important role in both visual and olfactory signal transduction. When associated with CNGA1, it is involved in the regulation of ion flow into the rod photoreceptor outer segment (ROS), in response to light-induced alteration of the levels of intracellular cGMP.

The term “proliferation” as used herein refers to an increase in the number of cells as a result of cell growth and cell division which may lead to either increased or decreased cell proliferation. Extensive cell proliferation occurs with hyperproliferative disorders, wherein the cell division of the cells is increased in relation to normal tissue. Such disorders are characterized by an abnormal proliferation (production) i.e. overproduction of cells. Hyperproliferative disorders comprise tumor diseases. Tumor diseases may comprise benign or malignant tumors wherein malignant tumor diseases are referred to as cancer. The term hyperproliferative disorder comprises cancers as well as pre-cancerous disorders. In particular embodiments the hyperproliferative disorders are hyperproliferative disorders of rod cells, in particular retinoblastoma.

The term “amino acid” generally refers to any monomer unit that comprises a substituted or unsubstituted amino group, a substituted or unsubstituted carboxy group, and one or more side chains or groups, or analogs of any of these groups. As used herein, the term “amino acid” includes the following twenty natural or genetically encoded alpha-amino acids: alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V). In cases where “X” residues are undefined, these should be defined as “any amino acid.” The structures of these twenty natural amino acids are shown in, e.g., Stryer et al., Biochemistry, 5th ed., Freeman and Company (2002). Additional amino acids, such as selenocysteine and pyrrolysine, can also be genetically coded for (Stadtman (1996) “Selenocysteine,” Annu Rev Biochem. 65:83-100 and Ibba et al. (2002) “Genetic code: introducing pyrrolysine,” Curr Biol. 12 (13): R464-R466). Amino acids can be linked by peptide bonds to form peptides or polypeptides.

In the context of the present invention, the term “peptide” refers to a short polymer of amino acids linked by peptide bonds. It has the same chemical (peptide) bonds as proteins, but is commonly shorter in length. The shortest peptide is a dipeptide, consisting of two amino acids joined by a single peptide bond. There can also be a tripeptide, tetrapeptide, pentapeptide, etc. Typically, a peptide has a length of up to 8, 10, 12, 15, 18 or 20 amino acids. A peptide has an amino end and a carboxyl end, unless it is a cyclic peptide.

In the context of the present invention, the term “polypeptide” refers to a single linear chain of amino acids bonded together by peptide bonds and typically comprises at least about 21 amino acids. A polypeptide can be one chain of a protein that is composed of more than one chain or it can be the protein itself if the protein is composed of one chain.

The term “fragment” used herein refers to naturally occurring fragments (e.g. splice variants) as well as artificially constructed fragments, in particular to those obtained by gene-technological means. Typically, a fragment has a deletion of up to 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, or 300 amino acids at its N-terminus and/or at its C-terminus and/or internally as compared to the parent polypeptide, preferably at its N-terminus, at its N- and C-terminus, or at its C-terminus.

As used herein, the term “variant” is to be understood as a polypeptide or polynucleotide which differs in comparison to the polypeptide or polynucleotide from which it is derived by one or more changes in its length or sequence. The polypeptide or polynucleotide from which a polypeptide or polynucleotide variant is derived is also known as the parent polypeptide or polynucleotide. The term “variant” comprises “fragments” or “derivatives” of the parent molecule. Typically, “fragments” are smaller in length or size than the parent molecule, whilst “derivatives” exhibit one or more differences in their sequence in comparison to the parent molecule. Also encompassed are modified molecules such as but not limited to post-translationally modified proteins (e.g. glycosylated, biotinylated, phosphorylated, ubiquitinated, palmitoylated, or proteolytically cleaved proteins) and modified nucleic acids such as methylated DNA. Also mixtures of different molecules such as but not limited to RNA-DNA hybrids, are encompassed by the term “variant”. Typically, a variant is constructed artificially, preferably by gene-technological means, whilst the parent protein or polynucleotide is a wild-type protein or polynucleotide, or a consensus sequence thereof. However, also naturally occurring variants are to be understood to be encompassed by the term “variant” as used herein. Further, the variants usable in the present invention may also be derived from homologs, orthologs, or paralogs of the parent molecule or from artificially constructed variant, provided that the variant exhibits at least one biological activity of the parent molecule, i.e. is functionally active.

In particular, the term “peptide variant”, or “polypeptide variant” is to be understood as a peptide, polypeptide, or protein which differs in comparison to the peptide, polypeptide, or protein from which it is derived by one or more changes in the amino acid sequence. The peptide, polypeptide, or protein, from which a peptide, polypeptide, or protein variant is derived, is also known as the parent peptide, polypeptide, or protein. Further, the variants usable in the present invention may also be derived from homologs, orthologs, or paralogs of the parent peptide, polypeptide, or protein or from artificially constructed variant, provided that the variant exhibits at least one biological activity of the parent peptide, polypeptide, or protein. The changes in the amino acid sequence may be amino acid exchanges, insertions, deletions, N-terminal truncations, or C-terminal truncations, or any combination of these changes, which may occur at one or several sites. A peptide, polypeptide, or protein variant may exhibit a total number of up to 200 (up to 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, or 200) changes in the amino acid sequence (i.e. exchanges, insertions, deletions, N-terminal truncations, and/or C-terminal truncations). The amino acid exchanges may be conservative and/or non-conservative. Alternatively or additionally, a “variant” as used herein, can be characterized by a certain degree of sequence identity to the parent peptide, polypeptide, or protein from which it is derived. More precisely, a peptide, polypeptide, or protein variant in the context of the present invention exhibits at least 80% sequence identity to its parent peptide, polypeptide, or protein. The sequence identity of peptide, polypeptide, or protein variants is over a continuous stretch of 20, 30, 40, 45, 50, 60, 70, 80, 90, 100 or more amino acids.

The “percentage of sequences identity” is determined by comparing two optimally aligned sequences over a comparison window, wherein the portion of the sequence in the comparison window can comprise additions or deletions (i.e. gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “identical” in the context of two or more nucleic acids or polypeptide sequences, refers to two or more sequences or subsequences that are the same, i.e. comprise the same sequence of nucleotides or amino acids. Sequences are “substantially identical” to each other if they have a specified percentage of nucleotides or amino acid residues that are the same (e.g., at least 70%, at least 75%, at least 80, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity over the aligned region), when compared and aligned for maximum correspondence over a comparison window, or designated region as measured using one of the following sequence comparison algorithms or by manual alignment and visual inspection. These definitions also refer to the complement of a test sequence. Accordingly, the term “at least 80% sequence identity” is used throughout the specification with regard to polypeptide and polynucleotide sequence comparisons. This expression preferably refers to a sequence identity of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to the respective reference polypeptide or to the respective reference polynucleotide.

The term “sequence comparison” refers to the process wherein one sequence acts as a reference sequence, to which test sequences are compared. When using a sequence comparison algorithm, test and reference sequences are entered into a computer, if necessary subsequence coordinates are designated, and sequence algorithm program parameters are designated. Default program parameters are commonly used, or alternative parameters can be designated. The sequence comparison algorithm then calculates the percent sequence identities or similarities for the test sequences relative to the reference sequence, based on the program parameters. In case where two sequences are compared and the reference sequence is not specified in comparison to which the sequence identity percentage is to be calculated, the sequence identity is to be calculated with reference to the longer of the two sequences to be compared, if not specifically indicated otherwise. If the reference sequence is indicated, the sequence identity is determined on the basis of the full length of the reference sequence indicated by SEQ ID, if not specifically indicated otherwise.

“Operably linked” as used in the context of the present invention refers to an arrangement of elements, wherein the components so described are configured so as to perform their usual function. A nucleic acid is “operably linked” when it is placed into a functional relationship with another nucleic acid sequence. For example, a promoter is operably linked to one or more transgenes, if it affects the transcription of the one or more transgenes. Further, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

The term “polyadenylation signal” (PAS) as used herein refers to a sequence involved in the process of mature messenger RNA (mRNA) production for translation. It, therefore, forms part of the larger process of gene expression. The process of polyadenylation begins as the transcription of a gene terminates. The 3′-most segment of the newly made pre-mRNA is first cleaved off by a set of proteins; these proteins then synthesize the poly (A) tail at the RNA's 3′ end. In some genes these proteins add a poly(A) tail at one of several possible sites. Therefore, polyadenylation can produce more than one transcript from a single gene (alternative polyadenylation), similar to alternative splicing. The poly(A) tail is important for the nuclear export, translation, and stability of mRNA. The tail is shortened over time, and, when it is short enough, the mRNA is enzymatically degraded. However, in a few cell types, mRNAs with short poly(A) tails are stored for later activation by re-polyadenylation in the cytosol. The PAS of the present invention may comprise a nucleic acid encoding a short Simian-Virus 40 (SV40) poly adenylation signal (SV 40 PAS). This modification of the polynucleotide has the advantage that expression of the gene of interest, for example the hCNGB1 in photoreceptor cells is significantly enhanced. The long-term expression that is achieved by the inclusion of SV40 PAS qualifies the polynucleotide for its use as an active gene therapy agent. In particular, the PAS can comprise the nucleic acid sequence according to SEQ ID NO: 4.

As used in this specification the term “vector”, also referred to as an expression construct, is usually a virus designed for protein expression in cells. The term “vector” refers to a protein or a polynucleotide or a mixture thereof which is capable of being introduced or of introducing proteins and/or nucleic acids comprised therein into a cell. Examples of vectors include but are not limited to plasmids, cosmids, phages, viruses or artificial chromosomes. In particular, a vector is used to transport the promoter and transgene of the invention into a suitable host cell. Vectors may contain “replicon” polynucleotide sequences that facilitate the autonomous replication of the vector in a host cell. Foreign DNA is defined as heterologous DNA, which is DNA not naturally found in the host cell, which, for example, replicates the vector molecule, encodes a selectable or screenable marker, or encodes a transgene. Once in the host cell, the vector can replicate independently of or coincidental with the host chromosomal DNA, and several copies of the vector and its inserted DNA can be generated. In addition, the vector can also contain the necessary elements that permit transcription of the inserted DNA into an mRNA molecule or otherwise cause replication of the inserted DNA into multiple copies of RNA. Vectors may further encompass “expression control sequences” that regulate the expression of the gene of interest. Typically, expression control sequences are polypeptides or polynucleotides such as but not limited to promoters, enhancers, silencers, insulators, or repressors. In a vector comprising more than one polynucleotide encoding for one or more gene products of interest, the expression may be controlled together or separately by one or more expression control sequences. More specifically, each polynucleotide comprised on the vector may be control by a separate expression control sequence or all polynucleotides comprised on the vector may be controlled by a single expression control sequence. Polynucleotides comprised on a single vector controlled by a single expression control sequence may form an open reading frame. Some expression vectors additionally contain sequence elements adjacent to the inserted DNA that increase the half-life of the expressed mRNA and/or allow translation of the mRNA into a protein molecule. Many molecules of mRNA and polypeptide encoded by the inserted DNA can thus be rapidly synthesized.

The term “AAV vector” as used in the context of the present invention refers to a complete virus particle, i.e., including a linear, single-stranded AAV nucleic acid genome associated with an AAV capsid protein coat. In this regard, single-stranded AAV nucleic acid molecules of either complementary sense (i.e., “sense” or “antisense” strands) can be packaged into any one AAV virion; both strands are equally infectious. The AAV vector of the present invention may also be an infectious and replication-defective virus composed of an AAV protein shell, encapsidating a heterologous DNA molecule of interest (e.g., hCNGB1) which may be flanked on both sides by an AAV ITR. An exemplary AAV 5′ ITR has the nucleic acid sequence according to SEQ ID NO: 5 and an exemplary AAV 3′ ITR has the nucleic acid sequence of the complement of SEQ ID NO: 6.An AAV vector of the present invention may be produced in a suitable host cell which has had an AAV vector, AAV helper functions and accessory functions introduced therein. In this manner, the host cell is rendered capable of encoding AAV polypeptides that are required for packaging the AAV genome (i.e., containing a recombinant nucleotide sequence of interest) into recombinant virion particles for subsequent gene delivery.

Various naturally occurring serotypes of adeno-associated virus (AAV), including 12 human serotypes (AAV1, AAV2, AAV3, AAV4, AAV5, AAV6, AAV7, AAV8, AAV9, AAV10, AAV11, and AAV12) and several serotypes from nonhuman primates have been identified. The different AAV serotypes also differ in their genome sequence, e.g. in the sequence of the inverted terminal repeats (ITRs) or the sequence encoding the capsid. The term “AAV genome” as used in the context of the present invention refers to any nucleic acid sequence derived from an adeno-associated virus serotype, including, without limitation, AAV-1, AAV-2, AAV-3, AAV-4, AAV-5, AAV-9, AAV-7, etc. AAV genome can have one or more of the AAV wild-type genes deleted in whole or in part, preferably the Rep and/or Cap genes, but retain functional flanking inverted terminal repeat (“ITR”) sequences. Functional ITR sequences are generally necessary for the rescue, replication and packaging of the AAV genome. Thus, an AAV genome is defined herein to include at least those sequences required in cis for replication and packaging (e.g., functional ITRs) of the virus. The ITRs need not be the wild-type nucleotide sequences, and may be altered (e.g., by the insertion, deletion or substitution of nucleotides) so long as the sequences provide for functional rescue, replication and packaging. The ITRs may comprise sequences according to SEQ ID NO: 5 and/or SEQ ID NO: 6.

“Antibodies” as used in the context of the present invention are glycoproteins belonging to the immunoglobulin superfamily; the terms antibody and immunoglobulin are often used interchangeably. An antibody refers to a protein molecule produced by plasma cells and is used by the immune system to identify and neutralize foreign objects such as bacteria and viruses. The antibody recognizes a unique part of the foreign target, its antigen.

The term “antibody binding fragment” as used herein, refers to one or more fragments of an antibody that retain the ability to specifically bind to an antigen. Examples of binding fragments encompassed within the term “antibody binding fragment” include a fragment antigen binding (Fab) fragment, a Fab′ fragment, a F(ab′)₂ fragment, a heavy chain antibody, a single-domain antibody (sdAb), a single-chain fragment variable (scFv), a fragment variable (Fv), a V_{H} domain, a V_{L} domain, a single domain antibody, a nanobody, an IgNAR (immunoglobulin new antigen receptor), a di-scFv, a bispecific T-cell engager (BITEs), a dual affinity re-targeting (DART) molecule, a triple body, a diabody, a single-chain diabody, an alternative scaffold protein, and a fusion protein thereof.

The term “pharmaceutical composition” as used in the present application include the formulation of the active compound or ingredient, i.e. the polynucleotide, the plasmid and/or the vector of the present invention and refers to a substance and/or a combination of substances being used for the identification, prevention, maintenance or treatment of a tissue status or disease. The pharmaceutical composition is formulated to be suitable for administration to a patient in order to prevent and/or treat disease and/or maintain the physiological state. Further a pharmaceutical composition refers to the combination of an active agent with a carrier, inert or active, making the composition suitable for therapeutic use. Pharmaceutical compositions can be formulated for oral, parenteral, topical, inhalative, rectal, sublingual, transdermal, subcutaneous or vaginal application routes according to their chemical and physical properties. Pharmaceutical compositions comprise solid, semisolid, liquid, transdermal therapeutic systems (TTS). Solid compositions are selected from the group consisting of tablets, coated tablets, powder, granulate, pellets, capsules, effervescent tablets or transdermal therapeutic systems. Also comprised are liquid compositions, selected from the group consisting of solutions, syrups, infusions, extracts, solutions for intravenous application, solutions for infusion or solutions of the carrier systems of the present invention. Semisolid compositions that can be used in the context of the invention comprise emulsion, suspension, creams, lotions, gels, globules, buccal tablets and suppositories.

“Pharmaceutically acceptable” means approved by a regulatory agency of the Federal or a state government or listed in the U.S. Pharmacopeia or other generally recognized pharmacopeia for use in animals, and more particularly in humans.

A “carrier” as referred to within this specification comprises a composition capable of delivering a reagent to a desired compartment, e.g. a certain cell type, of the human body and is useful for providing and controlling release of drugs after being administered by the chosen administration route and scheme.

As used in herein the route of administration describes the uptake of a xenobiotic in the human body and is classified by the location at which the xenobiotic is applied. The pharmaceutical composition comprising the polynucleotide and/or the viral vector, in particular in the method of treatment, is selected from intraocular, intrabulbar, intravitreal or subretinal

The term “disease” refers to an abnormal condition, especially an abnormal medical condition such as an illness or injury, wherein a cell, a tissue, an organ, or an individual is not able to efficiently fulfil its function anymore. Typically, but not necessarily, a disease is associated with specific symptoms or signs indicating the presence of such disease. The presence of such symptoms or signs may thus, be indicative for a cell, a tissue, an organ, or an individual suffering from a disease. An alteration of these symptoms or signs may be indicative for the progression of such a disease. A progression of a disease is typically characterised by an increase or decrease of such symptoms or signs which may indicate a “worsening” or “bettering” of the disease. The “worsening” of a disease is characterised by a decreasing ability of a cell, tissue, organ or individual/patient to fulfil its function efficiently, whereas the “bettering” of a disease is typically characterised by an increase in the ability of a cell, tissue, an organ or an individual/patient to fulfil its function efficiently. A cell, a tissue, an organ or an individual being “susceptible” to a disease is in a healthy state but especially vulnerable to the emergence of a disease, e.g. due to genetic predisposition, lacking vaccination, poorly developed or immature immunity, poor nutritional status, or the like.

A “disease of the retina” in the context of the present invention refers but is not limited to any kind of retinal degeneration. Retinal dystrophies, belonging to the group of retinal degenerations, are a broad group of genetic retinal disorders of varying severity and with differing inheritance patterns. A retinal dystrophy belongs to the group of pigmentary retinopathies. Retinitis Pigmentosa is the most common retinal dystrophy and is characterized by retinal pigment deposits visible on fundus examination and primary loss of rod photoreceptor cells followed by secondary loss of cone photoreceptors. Patients typically have night vision blindness and loss of midperipheral visual field. As the condition of the disease progresses, patients suffering the disease lose their far peripheral visual field and eventually central vision as well. The retinal degeneration may be associated with a genetic mutation, substitution, and/or deletion. The retinal degeneration is selected from the group consisting of night blindness, blindness, retinal degeneration, retinal dystrophy and Retinitis Pigmentosa. The Retinitis Pigmentosa can be CNGB1-linked Retinitis Pigmentosa or Retinitis Pigmentosa type 45 (RP45).

Other examples of retinal disorders include, without limitation, RPE65-mediated retinal disorders, macular degeneration (e.g., age-related macular degeneration), inherited juvenile macular degeneration (e.g., Stargardt disease), Rod-cone dystrophy, Cone-rod dystrophy, Oguchi disease, Malattia Leventinese, and others.

As used herein, “CNGB1-linked Retinitis Pigmentosa” refers to a class of diseases involving progressive degeneration of the retina, typically starting in the mid-periphery and advancing toward the macula and fovea (Ferrani et al. (2011) Curr. Genomics 12 (4): 238). Typical phenotypic symptoms include night blindness followed by decreasing visual fields, leading to tunnel vision and eventually legal blindness or, in many cases, complete blindness. On the cellular level, this correlates with a predominantly affected rod photoreceptor system. In later stages, the disease may further affect the cone photoreceptor eventually causing complete blindness. The diseased photoreceptors undergo apoptosis, which is reflected in reduced outer nuclear layer thickness within the retina, as well as in lesions and/or retinal pigment deposits in the fundus. Patients may lose a significant portion of their photoreceptors before experiencing loss of visual acuity. Clinical phenotypical hallmarks include, but are not limited to: (i) an abnormal fundus with bone-spicule deposits and attenuated retinal vessels; (ii) abnormal, diminished or absent a- and b-waves in the electroretinogram (ERG); and (iii) reduced visual field. Symptoms typically start in the early teenage years and severe visual impairment occurs by ages 40 to 50 years.

An example of a genetic variation that is known to be pathogenic for Retinitis Pigmentosa is a homozygous splice site mutation at the donor site of exon 32 of the CNGB1 gene (3444+1G-A) that results in a frameshift and truncation of the last 28 amino acids. Another example of a genetic variation that is known to be pathogenic for Retinitis Pigmentosa is a homozygous 2978G-T transversion in exon 30 of the CNGB1 gene that is predicted to result in a Glycine to Valine substitution at position 993 of the protein (G993V). Glycine 993 of CNGB1 is a conserved residue. Another example of a genetic variation that is known to be pathogenic for Retinitis Pigmentosa is a homozygous c. 1589C-G transversion in the CNGB1 gene, resulting in a proline to arginine substitution at position 530 of the CNGB1 protein (P530R). Another known genetic variation that is pathogenic for Retinitis Pigmentosa includes a c.2128C-T change in the CNGB1 gene, resulting in a Glutamine to Termination substitution at position 710 of the CNGB1 protein (Q710Stop). Other genetic variations that are pathogenic for Retinitis Pigmentosa can be found in the Online Mendelian Inheritance in Man (OMIM) database, and the ClinVar database maintained by the National Center for Biotechnology Information, incorporated herein by reference in their entirety for all purposes.

CNGB1-linked Retinitis Pigmentosa can be identified with methods known in the art to detect one or more phenotypic signs described herein, and/or one or more genetic variations in the CNGB1 gene. Any genetic variation that results in a change in a conserved residue of CNGB1 may be pathogenic for Retinitis Pigmentosa.

As used herein, “treat,” “treating,” “treatment,” or “therapy” of a disease or disorder means accomplishing one or more of the following: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in an individual that has previously had the disorder(s); and (e) limiting or preventing recurrence of symptoms in individuals that were previously symptomatic for the disorder(s).

### EMBODIMENTS

In the following different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

In gene therapeutic and/or gene corrective therapy approaches in which nucleic acids are introduced into cells, e.g., to augment expression, replace a defective gene, and/or inhibit expression of a defective gene, it is generally desirable that all transgenic elements are small. Nevertheless, it is often difficult to identify transgenic elements that can be reduced in size without negatively effecting or losing their activity in the in vivo situation. Generally, in vitro experiments are not suitable to indicate the in vivo behaviour of the elements making the determination of possible size reductions difficult and unpredictable. In the work leading to the present invention, it was surprisingly shown that a short part of the human rod promoter, i.e. an element smaller than 200 bases, could transfer rod-photoreceptor specific expression on transgenes operably linked to this promoter element in vivo. When the promoter element defined herein was used in an in vivo setting, stable integration and expression of a transgene was observed. The expression level was suitable to improve the visual capabilities of the test animals transfected with an adeno-associated virus vector comprising the transgene.

This surprising finding provides inter alia the following advantages over the art: (i) reduction of the size of the construct that is introduced into a cell, (ii) an increase of the packaging efficiency of the transgene into viral vectors, (iii) a decrease of the chance that recombination events occur in vivo, (iv) increase the efficiency of introduction of the transgene into the target cells, in particular into the nucleus of the target cell; (v) a suitable expression level in a human patient to treat rod associated diseases, (vi) preservation and/or improvement of retinal function in vivo and/or (vii) preservation and/or improvement of vision in vivo.

In a first aspect the present invention relates to a polynucleotide comprising in this order:
a) promoter comprising a human rod photoreceptor-specific promoter element (hRPSPE) comprising, consisting essentially of or consisting of the nucleic acid sequence according to SEQ ID NO: 1 or variants thereof and a core promoter (CP); and
b) at least one transgene (TG) operably linked to the promoter of a);
wherein the variant of SEQ ID NO: 1 comprises one or more nucleic acid substitutions outside nucleotide positions 6 to 13, 32 to 40, 70 to 83, and 87 to 94 of SEQ ID NO: 1 and wherein the length of the promoter is in particular 350 bases or less. The promoter that provides one or more of above advantages may also be longer than 350 bases, e.g. 600 bp or less, 500 bp or less, or 400 bp or less. In particular embodiments the promoter has a length of 300 bases or less, in other embodiments it has a length of 300 bases or less, in other embodiments it has a length of 250 bases or less, in other embodiments it has a length of 200 bases or less, in other embodiments it has a length of 194 bases or less.

In an attempt to minimize the overall length of heterologous bases introduced into a patient, the polynucleotide comprises no other human rod promoter and/or gene nucleotide sequence other than expressly defined in a) above.

The indicated nucleotides are to be preserved in variants of SEQ ID NO: 1 since the present inventors believe that these nucleotide sequences are instrumental in conferring rod photoreceptor-specific expression to the hRPSPE. Outside the putative transcription factor binding sequences (TFBs) 1 or more nucleotides can be mutated or inserted. If nucleotides are inserted, the insertion of 1 to 70 nucleotides is an advantageous number with multiples of seven being particularly advantageous since this number maintains the relative rotational positions of the TFBs. It is, however advantageous, if the distance between the TFBs is not altered to avoid rotational displacement of the transcription factors binding to the promoter element. Thus, within the 99 bp long sequence according to SEQ ID NO: 1 it is permissible to mutate one or more nucleotides at positions 1 to 5, 14 to 31, 41 to 69, 84 to 86 and 95 to 99. Thus, maximally 50 nucleotides may be mutated within SEQ ID NO: 1. Accordingly, particular variants comprise between 1 to 50 mutations, i.e. 1, 2, 3, 4, 5, 6, 7, 8, 9, 10, 11, 12, 13, 14, 15, 16, 17, 18,19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. Other particular variants comprise between 5 to 40, between 10 to 30 or between 20 to 25 mutations. The promoter comprising a variant of the hRPSPE shows a rod photoreceptor-specific expression level as a promoter comprising the hRPSPE comprising the nucleic acid sequence according to SEQ ID NO: 1, preferably a promoter consisting of nucleotides 155 to 350 or 155 to 348 of SEQ ID NO: 2. It is advantageous, if the variant shows at least 10% of the expression level of a promoter consisting of nucleotides 155 to 350 or 155 to 348 of SEQ ID NO: 2. Other advantageous expression levels are at least 20%, at least 30%, at least 40%, at least 50%, at least 60%, at least 70%, at least 80%, at least 90% or at least 100%. Different expression levels may be advantageous depending on the respective therapeutic approach, in particular lower expression levels than those obtained with a promoter consisting of nucleotides 155 to 350 of SEQ ID NO: 2 may be advantageous, if a higher expression level of the transgene overcompensates the deficiency or leads to deleterious effect.

It is also envisioned that the polynucleotide of the invention comprises two or more transgenes operably linked to the promoter of a). In such a situation separate expression of the two or more transgenes can be obtained by inserting a nucleotide sequence allowing separate translation of the two transgenes, e.g. encoding an Internal Ribosomal Entry Site (IRES), between the two transgenes.

In an embodiment of the first aspect of the invention the 5′ end of the hRPSPE comprised in the promoter of a) is at a nucleic acid position from 1 to 160 and the 3′ end at a nucleic acid position from 290 to 310 of SEQ ID NO: 2 or variants thereof. In a particular embodiment the 5′ end of the hRPSPE is at one of the following nucleic acid positions: 1, 10, 20, 30, 40, 50, 60, 70, 80, 90, 100, 110, 120, 130, 140, 145, 150, 151, 152, 153, 154, 155, 156, 157, 158, 159 or 160 of SEQ ID NO: 2. In a particular embodiment the 3′ end of the hRPSPE is at one of the following nucleic acid positions: 290, 291, 292, 293, 294, 295, 296, 297, 298, 299, 300, 301, 302, 303, 304, 305, 306, 307, 308, 309, or 310 of SEQ ID NO: 2. Thus, according to particular embodiments the hRPSPE comprised in the promoter of a) spans nucleic acid positions 1 to 310, 10 to 309, 20 to 308, 30 to 307, 40 to 306, 50 to 305, 60 to 304, 70 to 303, 80 to 302, 90 to 301, 100 to 300, 110 to 299, 120 to 298, 130 to 297, 140 to 296, 150 to 295, 151 to 294, 152 to 293, 153 to 292, 154 to 291 or 155 to 290 of SEQ ID NO: 2. The term “variants of hRPSPE” has the meaning outlined above. Thus, variants of the fragments indicated in this paragraph have the respectively indicated 5′ and 3′ end and may additionally comprise mutations outside the sequences indicated above with reference to SEQ ID NO: 1.

In an embodiment of the first aspect of the invention the CP comprises a TATA-box and/or an initiator (Inr). In a particular embodiment the TATA-box and Inr of the human rho promoter. In a particular embodiment the 5′end of the CP comprised in the promoter of a) is at nucleotide position 300, 301, 302, 303, 304, 305, 306, 307, 308, 309, 310, 311, 312, 313, 314 of SEQ ID NO: 2 and the 3′ end is at nucleic acid position from 330, 331, 332, 333, 334, 335, 336, 337, 338, 339, 340, 342, 343, 344, 345, 346, 347, 348, 349 or 350 of SEQ ID NO: 2. Thus, according to particular embodiments the CP comprised in the promoter of a) spans nucleic acid positions 300 to 350, 301 to 350, 302 to 350, 303 to 350, 304 to 349, 305 to 349, 306 to 349, 307 to 349, 308 to 348, 309 to 348, 310 to 348, 311 to 348, 312 to 348, 313 to 348, or 314 to 348 of SEQ ID NO: 2. In an embodiment of the first aspect of the invention the 5′ end of the promoter is at a nucleic acid position from 1 to 160 and the 3′ end at a nucleic acid position from 340 to 350 of SEQ ID NO: 2 or variants thereof. In a particular embodiment the 5′ end of the promoter is at one of the following nucleic acid positions: 1, 10, 20, 30, 40, 50, 60, 70, 80, 90, 100, 110, 120, 130, 140, 145, 150, 151, 152, 153, 154, 155, 156, 157, 158, 159 or 160. In a particular embodiment the 3′ end of the promoter is at one of the following nucleic acid positions: 330, 331, 332, 333, 334, 335, 336, 337, 338, 339, 340, 342, 343, 344, 345, 346, 347, 348, 349 or 350 of SEQ ID NO: 2. Thus, according to particular embodiments the promoter of a) spans nucleic acid positions 1 to 350, 10 to 350, 20 to 350, 30 to 350, 40 to 350, 50 to 350, 60 to 350, 70 to 350, 80 to 350, 90 to 349, 100 to 349, 110 to 349, 120 to 349, 130 to 349, 140 to 348, 150 to 348, 151 to 348, 152 to 348, 153 to 348, 154 to 348 or 155 to 348 of SEQ ID NO: 2. In a particular embodiment the promoter comprises, essentially consists or consists of SEQ ID NO: 9.

In an embodiment of the first aspect of the invention the transgene comprises, essentially consists or consists of a nucleic acid encoding a protein that maintains or improves the physiological function of rod cells and/or inhibits proliferation of rod cells. Typically, such genes are naturally expressed in healthy rod cells. The skilled person is aware of a large number of genes expressed in rod cells that are involved in the physiological function of rod cells. This function comprises inter alia, the detection of photons and the generation of nerve pulses in response to the detection of one or more photons.

In an embodiment of the first aspect of the invention the transgene:
(i) comprises a nucleic acid encoding the human rod cyclic nucleotide-gated channel beta subunit (hCNGB1), ABCA4, AIPL1, BEST1, CACNA1F, CLN3, CLRN1, CNGA1, CEP290, CRB1, CRB2, CRX, GPR98, GUCA1A, GUCA1B, MYO7A, NRL, PDE6A, PDE6B, PRPH2, PROM1, RHO, ROM1, RP1, RP2, RPE65, RPGR, SAG, USH1C, USH1G, USH2A or functional fragments or variants thereof; a nucleic acid encoding a miRNA or shRNA targeting a mRNA encoding a dominant negative mutant thereof; and/or a nucleic acid encoding an antibody or antibody binding fragment that specifically binds to a dominant negative mutant thereof; or
(ii) comprises a nucleic acid encoding a protein that inhibits proliferation of rod cells, preferably a toxin; a prodrug converting enzyme, e.g. thymidine kinase; cell cycle inhibitors, e.g. retinoblastoma protein (pRB), p53, p21CIP1, p27KIP1 and p57KIP2; comprises a mRNA encoding a dominant negative mutant of the cell cycle inhibitor thereof; and/or comprises a nucleic acid encoding a dominant negative mutant of a cell cycle inhibitor thereof.

Some diseases of rod cells are characterized by recessive mutations in one or more of the genes that maintain or improve the function of rod cells or that prevent hyperproliferation, in particular genes encoding the proteins indicated in (i) or (ii). In such cases it is often sufficient in order to cure or at least to ameliorate the disease, if a transgene encoding the functional protein is introduced into the rod cell, in particular using a vector. If the disease is however, caused by a dominant negative mutation the provision of a transgene encoding the functional protein or functional fragment thereof, is often not sufficient to cure or ameliorate the disease. In such cases it is preferred that the expression or function of the dominant negative mutant protein is reduced in the cell, i.e. is knocked-down. Such knock-down may be affected by expressing a transgene encoding an inhibitory RNA that specifically reduces expression of the dominant negative mutant protein or by one or more transgenes encoding an antibody of fragment thereof that specifically binds to and inactivates the dominant negative mutant protein and does not significantly bind to the corresponding functional protein. The skilled person is well aware how to design such inhibitory RNA specific to the mRNA encoding the respective dominant negative mutant protein. Similarly, the skilled person knows how to generate antibodies that specifically bind only to the dominant negative mutant protein and not to the wild-type protein. In its natural form antibodies comprise two different protein chains. Thus, if both protein chains of an antibody are expressed to knock-down a protein, then one transgene may comprise nucleotides encoding the light chain linked through an Internal Ribosomal Entry Site (IRES) to another transgene encoding the heavy chain. In this way both antibody chains can be expressed from a single mRNA. It is apparent to the skilled person that the order of light and heavy chain can be reversed without affecting expression of the antibody within the rod cell. Alternatively, a single chain antibody may be encoded by the transgene.

In a particular embodiment the diseases to be treated are characterized by dominant negative mutations in one or more of the genes that maintain or improve the function of rod cells or that promote hyperproliferation, in particular in one or more of AIPL1, BEST1, NR2E3, NRL, PRPH2, RHO, ROM1, and/or RP1. In this case it is preferred that expression and/or function of the proteins encoded by the dominant negative mutant gene is knocked down and that a transgene encoding the functional protein or a functional fragment thereof. If size limitations of the respective vector allows, it is preferred that the polynucleotide comprises both a transgene encoding the functional protein or a functional fragment thereof and a transgene encoding an inhibitory RNA or an inhibitory antibody or fragment thereof. If both transgenes encode proteins they can be under the control of the same promoter and use, e.g. an IRES sequence between the two transgenes or if one transgene encodes a protein and the other an inhibitory RNA each transgene may be operably linked to a separate promoter according to i) of the first aspect of the invention.

The term “functional fragments” refers to N- and/or C-terminal deletions of the respective protein that does not lead to a loss of the rod cell specific function of the respective proteins. The term “variants thereof” refers to proteins that have at least 70% sequence identity to the respectively indicated human wild-type protein, in particular the proteins according to SEQ ID NO: 3, 10 to 41, and 45. In a particular embodiment the variant has at least 70% sequence identity, particularly 75% sequence identity, more particularly 80% sequence identity, more particularly 85% sequence identity, more particularly 90% sequence identity, and more particularly 95% sequence identity to SEQ ID NO: 3. In a particular embodiment the variant has at least 70% sequence identity, particularly 75% sequence identity, more particularly 80% sequence identity, more particularly 85% sequence identity, more particularly 90% sequence identity, and more particularly 95% sequence identity to SEQ ID NO: 10. In a particular embodiment the variant has at least 70% sequence identity, particularly 75% sequence identity, more particularly 80% sequence identity, more particularly 85% sequence identity, more particularly 90% sequence identity, and more particularly 95% sequence identity to SEQ ID NO: 11. In a particular embodiment the variant has at least 70% sequence identity, particularly 75% sequence identity, more particularly 80% sequence identity, more particularly 85% sequence identity, more particularly 90% sequence identity, and more particularly 95% sequence identity to SEQ ID NO: 12. In a particular embodiment the variant has at least 70% sequence identity, particularly 75% sequence identity, more particularly 80% sequence identity, more particularly 85% sequence identity, more particularly 90% sequence identity, and more particularly 95% sequence identity to SEQ ID NO: 13. In a particular embodiment the variant has at least 70% sequence identity, particularly 75% sequence identity, more particularly 80% sequence identity, more particularly 85% sequence identity, more particularly 90% sequence identity, and more particularly 95% sequence identity to SEQ ID NO: 14. In a particular embodiment the variant has at least 70% sequence identity, particularly 75% sequence identity, more particularly 80% sequence identity, more particularly 85% sequence identity, more particularly 90% sequence identity, and more particularly 95% sequence identity to SEQ ID NO: 15. In a particular embodiment the variant has at least 70% sequence identity, particularly 75% sequence identity, more particularly 80% sequence identity, more particularly 85% sequence identity, more particularly 90% sequence identity, and more particularly 95% sequence identity to SEQ ID NO: 16. In a particular embodiment the variant has at least 70% sequence identity, particularly 75% sequence identity, more particularly 80% sequence identity, more particularly 85% sequence identity, more particularly 90% sequence identity, and more particularly 95% sequence identity to SEQ ID NO: 17. In a particular embodiment the variant has at least 70% sequence identity, particularly 75% sequence identity, more particularly 80% sequence identity, more particularly 85% sequence identity, more particularly 90% sequence identity, and more particularly 95% sequence identity to SEQ ID NO: 18. In a particular embodiment the variant has at least 70% sequence identity, particularly 75% sequence identity, more particularly 80% sequence identity, more particularly 85% sequence identity, more particularly 90% sequence identity, and more particularly 95% sequence identity to SEQ ID NO: 19. In a particular embodiment the variant has at least 70% sequence identity, particularly 75% sequence identity, more particularly 80% sequence identity, more particularly 85% sequence identity, more particularly 90% sequence identity, and more particularly 95% sequence identity to SEQ ID NO: 20. In a particular embodiment the variant has at least 70% sequence identity, particularly 75% sequence identity, more particularly 80% sequence identity, more particularly 85% sequence identity, more particularly 90% sequence identity, and more particularly 95% sequence identity to SEQ ID NO: 21. In a particular embodiment the variant has at least 70% sequence identity, particularly 75% sequence identity, more particularly 80% sequence identity, more particularly 85% sequence identity, more particularly 90% sequence identity, and more particularly 95% sequence identity to SEQ ID NO: 22. In a particular embodiment the variant has at least 70% sequence identity, particularly 75% sequence identity, more particularly 80% sequence identity, more particularly 85% sequence identity, more particularly 90% sequence identity, and more particularly 95% sequence identity to SEQ ID NO: 23. In a particular embodiment the variant has at least 70% sequence identity, particularly 75% sequence identity, more particularly 80% sequence identity, more particularly 85% sequence identity, more particularly 90% sequence identity, and more particularly 95% sequence identity to SEQ ID NO: 24. In a particular embodiment the variant has at least 70% sequence identity, particularly 75% sequence identity, more particularly 80% sequence identity, more particularly 85% sequence identity, more particularly 90% sequence identity, and more particularly 95% sequence identity to SEQ ID NO: 25. In a particular embodiment the variant has at least 70% sequence identity, particularly 75% sequence identity, more particularly 80% sequence identity, more particularly 85% sequence identity, more particularly 90% sequence identity, and more particularly 95% sequence identity to SEQ ID NO: 26. In a particular embodiment the variant has at least 70% sequence identity, particularly 75% sequence identity, more particularly 80% sequence identity, more particularly 85% sequence identity, more particularly 90% sequence identity, and more particularly 95% sequence identity to SEQ ID NO: 27. In a particular embodiment the variant has at least 70% sequence identity, particularly 75% sequence identity, more particularly 80% sequence identity, more particularly 85% sequence identity, more particularly 90% sequence identity, and more particularly 95% sequence identity to SEQ ID NO: 28. In a particular embodiment the variant has at least 70% sequence identity, particularly 75% sequence identity, more particularly 80% sequence identity, more particularly 85% sequence identity, more particularly 90% sequence identity, and more particularly 95% sequence identity to SEQ ID NO: 29. In a particular embodiment the variant has at least 70% sequence identity, particularly 75% sequence identity, more particularly 80% sequence identity, more particularly 85% sequence identity, more particularly 90% sequence identity, and more particularly 95% sequence identity to SEQ ID NO: 30. In a particular embodiment the variant has at least 70% sequence identity, particularly 75% sequence identity, more particularly 80% sequence identity, more particularly 85% sequence identity, more particularly 90% sequence identity, and more particularly 95% sequence identity to SEQ ID NO: 31. In a particular embodiment the variant has at least 70% sequence identity, particularly 75% sequence identity, more particularly 80% sequence identity, more particularly 85% sequence identity, more particularly 90% sequence identity, and more particularly 95% sequence identity to SEQ ID NO: 32. In a particular embodiment the variant has at least 70% sequence identity, particularly 75% sequence identity, more particularly 80% sequence identity, more particularly 85% sequence identity, more particularly 90% sequence identity, and more particularly 95% sequence identity to SEQ ID NO: 33. In a particular embodiment the variant has at least 70% sequence identity, particularly 75% sequence identity, more particularly 80% sequence identity, more particularly 85% sequence identity, more particularly 90% sequence identity, and more particularly 95% sequence identity to SEQ ID NO: 34. In a particular embodiment the variant has at least 70% sequence identity, particularly 75% sequence identity, more particularly 80% sequence identity, more particularly 85% sequence identity, more particularly 90% sequence identity, and more particularly 95% sequence identity to SEQ ID NO: 35. In a particular embodiment the variant has at least 70% sequence identity, particularly 75% sequence identity, more particularly 80% sequence identity, more particularly 85% sequence identity, more particularly 90% sequence identity, and more particularly 95% sequence identity to SEQ ID NO: 36. In a particular embodiment the variant has at least 70% sequence identity, particularly 75% sequence identity, more particularly 80% sequence identity, more particularly 85% sequence identity, more particularly 90% sequence identity, and more particularly 95% sequence identity to SEQ ID NO: 37. In a particular embodiment the variant has at least 70% sequence identity, particularly 75% sequence identity, more particularly 80% sequence identity, more particularly 85% sequence identity, more particularly 90% sequence identity, and more particularly 95% sequence identity to SEQ ID NO: 38. In a particular embodiment the variant has at least 70% sequence identity, particularly 75% sequence identity, more particularly 80% sequence identity, more particularly 85% sequence identity, more particularly 90% sequence identity, and more particularly 95% sequence identity to SEQ ID NO: 39. In a particular embodiment the variant has at least 70% sequence identity, particularly 75% sequence identity, more particularly 80% sequence identity, more particularly 85% sequence identity, more particularly 90% sequence identity, and more particularly 95% sequence identity to SEQ ID NO: 40. In a particular embodiment the variant has at least 70% sequence identity, particularly 75% sequence identity, more particularly 80% sequence identity, more particularly 85% sequence identity, more particularly 90% sequence identity, and more particularly 95% sequence identity to SEQ ID NO: 41. In a particular embodiment the variant has at least 70% sequence identity, particularly 75% sequence identity, more particularly 80% sequence identity, more particularly 85% sequence identity, more particularly 90% sequence identity, and more particularly 95% sequence identity to SEQ ID NO: 45.

Functional fragments of the above indicated proteins are those fragments that maintain the function of the respective protein in normally functioning rod photoreceptor, in particular in detecting photons and/or transmitting the information on the detection of photons. Similarly, variants also maintain the function of the respective protein in normally functioning rod photoreceptors.

In an embodiment of the first aspect of the invention the hCNGB1 encoded by the transgene comprises an amino acid sequence according to SEQ ID NO: 3 or variants thereof. In an embodiment of the first aspect of the invention the hCNGB1 encoded by the transgene comprises an amino acid sequence according to SEQ ID NO: 40 or variants thereof. In an embodiment of the first aspect of the invention the hCNGB1 encoded by the transgene comprises an amino acid sequence according to SEQ ID NO: 41 or variants thereof.

In an embodiment of the first aspect of the invention the polynucleotide comprises one or more further nucleotide sequence elements selected from the group consisting of:
(i) a polyadenylation signal (pA); and/or
(ii) one or two inverted terminal repeat (ITR) sequences; and/or
(iii) viral nucleotide sequences necessary to form an infectious viral vector, preferably an adeno-associated virus, an adenovirus, a retrovirus, a lentivirus, a vaccinia/poxvirus, or a herpesvirus vector, in particular herpes simplex virus (HSV) vector.

Viral nucleotide sequences that are essential to forming an infectious viral vector of the respective type are well known in the art. Any of these elements may be comprised in the polynucleotide of the first aspect of the invention.

In an embodiment of the first aspect of the invention the polyadenylation signal comprises, essentially consists or consists of a Simian-Virus 40 PAS.

In an embodiment of the first aspect of the invention the polyadenylation signal comprises, essentially consists or consists of a nucleic acid according to SEQ ID NO: 4 or functional variants thereof.

In an embodiment of the first aspect of the invention the ITR sequence is an adeno-associated virus (AAV) ITR.

In an embodiment of the first aspect of the invention the AAV ITR is of an AVV serotype 2, 5, 8 or 9.

In an embodiment of the first aspect of the invention the promoter and the transgene are flanked at their 5′ with a L-ITR and at their 3′ end with a R-ITR. In one particular embodiment the elements are arranged in 5′ to 3′ direction in the following order: L-ITR-promoter-transgene-R-ITR, L-ITR-transgene-promoter-R-ITR, R-ITR-promoter-transgene-L-ITR, or R-ITR-transgene-promoter-L-ITR. In another particular embodiment the elements are arranged in 5′ to 3′ direction in the following order: L-ITR-promoter-transgene-PAS-R-ITR, L-ITR-PAS-transgene-promoter-R-ITR, R-ITR-promoter-transgene-PAS-L-ITR, or R-ITR-PAS-transgene-promoter-L-ITR.

In an embodiment of the first aspect of the invention the L-ITR comprises, essentially consists or consists of a sequence according to SEQ ID NO: 5 or variants thereof and/or the R-ITR comprises, essentially consists or consists of a sequence according to SEQ ID NO: 6 or variants thereof.

Depending on the viral vector used the length of the nucleic acid that can be efficiently packaged in the viral vector greatly varies. Some vectors like adenoviral vectors can accommodate large nucleic acid inserts while others, like adeno-virus associated vectors efficiently package polynucleotides that have a length of 4700 bases or less. Irrespective of the nucleic acid packaging ability of a vector it is generally desirable to minimize the length of any heterologous nucleic acid introduced into a patient, in particular if the heterologous nucleic acid is stably introduced into the genome. Accordingly, in an embodiment of the first aspect of the invention the total length of the polynucleotide is 5200 bases or less, in particular 5100 bases or less, in particular 5000 bases or less, in particular 4900 bases or less, in particular 4800 bases or less, and more particular 4700 bases or less.

In a particular embodiment of the first aspect of the invention the polynucleotide comprises, essentially consists in 5′ to 3′ direction of the following nucleic acids elements: L-ITR-promoter-transgene-SV40 PAS-R-ITR, L-ITR-SV40 PAS-transgene-promoter-R-ITR, R-ITR-promoter-transgene-SV40 PAS-L-ITR, or R-ITR-SV40 PAS-transgene-promoter-L-ITR, wherein the transgene comprises, essentially comprises or consists of a nucleotide sequence encoding the hCNGB1 protein of SEQ ‘ID NO: 3, the PAS comprises, essentially comprises or consists of the nucleotide sequence of SEQ ID NO: 4, the L-ITR comprises, essentially comprises or consists of the nucleotide sequence SEQ ID NO: 5, the R-ITR comprises, essentially comprises or consists of the nucleotide sequence SEQ ID NO: 6, and the promoter comprises, essentially comprises or consists of the nucleotide sequence that spans nucleotides 155 to 348 of SEQ ID NO: 1. Also in this embodiment the total length of the polynucleotide is 5200 bases or less, in particular 5100 bases or less, in particular 5000 bases or less, in particular 4900 bases or less, in particular 4800 bases or less, and more particular 4700 bases or less.

A second aspect of the invention relates to a plasmid comprising the polynucleotide of the first aspect of the invention. A plasmid is a circular DNA that can be replicated in bacteria.

In an embodiment of the second aspect of the invention the plasmid comprises, essentially consists or consists of a nucleic acid sequence according to SEQ ID NO: 7 or variants thereof. In an embodiment of the second aspect of the invention the plasmid comprises, essentially consists or consists of a nucleic acid sequence according to SEQ ID NO: 42 or variants thereof. In an embodiment of the second aspect of the invention the plasmid comprises, essentially consists or consists of a nucleic acid sequence according to SEQ ID NO: 43 or variants thereof. In an embodiment of the second aspect of the invention the plasmid comprises, essentially consists or consists of a nucleic acid sequence according to SEQ ID NO: 44 or variants thereof.

A third aspect of the invention relates to a viral vector comprising the polynucleotide of the first aspect of the invention. In a particular embodiment the viral vector is an AAV, an adenovirus, a retrovirus, a lentivirus, a vaccinia/poxvirus, or a herpesvirus vector, in particular herpes simplex virus (HSV) vector. In a particular embodiment the viral vector is an AAV.

In an embodiment of the third aspect of the invention the virus is selected from the group consisting of AAV2, AAV5, AAV8, AVV9 or variants thereof.

A fourth aspect of the invention relates to the polynucleotide according to the first aspect of the invention, the plasmid of the second aspect of the invention and/or the viral vector according to the third aspect of the invention for use as a medicament.

A fifth aspect of the invention relates to a pharmaceutical composition comprising the polynucleotide according to the first aspect of the invention, the plasmid of the second aspect of the invention and/or the viral vector according to the third aspect of the invention, and a pharmaceutically acceptable carrier.

A sixth aspect of the invention relates to the polynucleotide according to the first aspect of the invention, the plasmid of the second aspect of the invention and/or the viral vector according to the third aspect of the invention for use in the therapy of a disease of the retina.

Advantageously the polynucleotide according to the first aspect of the invention, the plasmid of the second aspect of the invention and/or the viral vector according to the third aspect of the invention can be used in diseases that are associated with a loss of or aberrant rod receptor function, in particular retinal degeneration or hyperproliferation of rod cells, in particular retinoblastoma. While tissue specific expression of the transgene is obtained through the promoter of the polynucleotide of the first aspect of the invention and, thus systemic administration of the therapeutic polynucleotide, plasmid or viral vector can be systemic without and will still be limited to rod receptors it is more efficient, if the therapeutic polynucleotide, plasmid or viral vector of the invention is directly administered to the eye of the patient. Accordingly, particular routes of administration are selected from intraocular, intrabulbar, intravitreal or subretinal administration.

In an embodiment of the sixth aspect of the invention the retinal degeneration is associated with a genetic mutation, substitution, and/or deletion.

In an embodiment of the sixth aspect of the invention the retinal degeneration is associated with a genetic mutation, substitution, and/or deletion.

In an embodiment of the sixth aspect of the invention the degeneration is selected from the group consisting of night blindness, blindness, retinal degeneration, retinal dystrophy and retinitis pigmentosa.

In an embodiment of the sixth aspect of the invention the retinitis pigmentosa is CNGB1-linked retinitis pigmentosa or retinitis pigmentosa type 45 (RP45).

A seventh aspect of the invention relates to a polynucleotide comprising in this order:
a) a human rhodopsin promoter comprising the nucleic acid sequence according to SEQ ID NO: 9 or variants thereof; and
b) at least one transgene (TG) operably linked to the promoter of a).

In an embodiment of the seventh aspect of the invention, the transgene comprises a nucleic acid encoding a protein that maintains or improves a physiological function of rods.

In an embodiment of the seventh aspect of the invention the transgene:
(i) comprises a nucleic acid encoding the human rod cyclic nucleotide-gated channel beta subunit (hCNGB1), ABCA4, AIPL1, BEST1, CACNA1F, CLN3, CLRN1, CNGA1, CEP290, CRB1, CRB2, CRX, GPR98, GUCA1A, GUCA1B, MYO7A, NRL, PDE6A, PDE6B, PRPH2, PROM1, RHO, ROM1, RP1, RP2, RPE65, RPGR, SAG, USH1C, USH1G, USH2A or functional fragments or variants thereof; a nucleic acid encoding a miRNA or shRNA targeting a mRNA encoding a dominant negative mutant thereof; and/or a nucleic acid encoding an antibody or antibody binding fragment that specifically binds to a dominant negative mutant thereof; or
(ii) comprises a nucleic acid encoding a protein that inhibits proliferation of rod cells, preferably a toxin; a prodrug converting enzyme, e.g. thymidine kinase; cell cycle inhibitors, e.g. retinoblastoma protein (pRB), p53, p21CIP1, p27KIP1 and p57KIP2; comprises a mRNA encoding a dominant negative mutant of the cell cycle inhibitor thereof; and/or comprises a nucleic acid encoding a dominant negative mutant of a cell cycle inhibitor thereof.

In an embodiment of the seventh aspect of the invention, the polynucleotide further comprises one or more nucleotide sequence elements selected from the group consisting of:
(i) a polyadenylation signal (PAS);
(ii) one or two inverted terminal repeat (ITR) sequences; and
(iii) viral nucleotide sequences necessary to form an infectious viral vector, preferably an adenovirus, a retrovirus, a lentivirus, a vaccinia/poxvirus, or a herpesvirus vector, in particular herpes simplex virus (HSV) vector.

In an embodiment of the seventh aspect of the invention, the polyadenylation signal comprises a Simian-Virus 40 PAS.

In an embodiment of the seventh aspect of the invention, the ITR sequence is an adeno-associated virus (AAV) ITR.

In an embodiment of the seventh aspect of the invention, the AAV is AVV serotype 2, 5, 8 or 9.

An eighth aspect of the invention relates to a viral vector comprising the polynucleotide of the seventh aspect of the invention.

In an embodiment of the eighth aspect of the invention, the virus is selected from the group consisting of AAV2, AAV5, AAV8, AVV9 or variants thereof.

The polynucleotides of the invention comprising a human rod photoreceptor-specific promoter element (hRPSPE) or variants thereof and a core promoter (CP) operably linked to a transgene (e.g., CNGB1), or polynucleotides comprising a human rhodopsin promoter operably linked to a transgene (e.g., CNGB1) can be used in gene therapeutic and/or gene corrective therapies. In such therapies, the polynucleotides are introduced into cells to augment expression, replace a defective gene, and/or inhibit expression of a defective gene.

Accordingly, a ninth aspect of the invention relates to a method for treating retinal degeneration in a subject in need thereof, comprising administering to the subject a therapeutically effective amount of a polynucleotide according to a seventh aspect of the invention, or the viral vector according to an eighth aspect of the invention.

In an embodiment of the ninth aspect of the invention, the polynucleotide or viral vector comprises the nucleic acid sequence set forth in SEQ ID NO: 43.

A tenth aspect of the invention relates to a method for treating retinitis pigmentosa in a subject in need thereof, comprising administering to the subject a therapeutically effective amount of a polynucleotide according to a seventh aspect of the invention, or the viral vector according to an eighth aspect of the invention.

In an embodiment of the tenth aspect of the invention, the polynucleotide or viral vector comprises the nucleic acid sequence set forth in SEQ ID NO: 43.

An eleventh aspect of the invention relates to a method for treating retinal degeneration in a subject in need thereof, wherein the retinal degeneration is characterized by a defect or absence of CNGB1 in the retinal cells of the subject, the method comprising administering to the subject a therapeutically effective amount of a viral vector comprising the nucleic acid sequence set forth in SEQ ID NO: 43.

In an embodiment of the eleventh aspect of the invention, the retinal degeneration is CNGB1-linked retinitis pigmentosa or retinitis pigmentosa type 45 (RP45).

A twelfth aspect of the invention relates to a method for treating CNGB1-linked retinitis pigmentosa or retinitis pigmentosa type 45 (RP45) in a subject in need thereof, comprising subretinal administration to the subject a therapeutically effective amount of a viral vector comprising the nucleic acid sequence set forth in SEQ ID NO: 43.

A thirteenth aspect of the invention relates to a polynucleotide comprising in this order:
a) a promoter comprising a human rod photoreceptor-specific promoter element (hRPSPE) comprising the nucleic acid sequence according to SEQ ID NO: 1 or variants thereof and a core promoter (CP); and
b) a transgene encoding the human rod cyclic nucleotide-gated channel beta subunit (hCNGB1) operably linked to the promoter of a),
wherein the variant of SEQ ID NO: 1 comprises one or more nucleic acid substitutions outside nucleotide positions 6 to 13, 32 to 40, 70 to 83, and 87 to 94 of SEQ ID NO: 1.

A fourteenth aspect of the invention relates to a pharmaceutical composition comprising a polynucleotide comprising in this order:
a) a promoter comprising a human rod photoreceptor-specific promoter element (hRPSPE) comprising the nucleic acid sequence according to SEQ ID NO: 1 or variants thereof and a core promoter (CP); and
b) a transgene encoding the human rod cyclic nucleotide-gated channel beta subunit (hCNGB1) operably linked to the promoter of a);
wherein the variant of SEQ ID NO: 1 comprises one or more nucleic acid substitutions outside nucleotide positions 6 to 13, 32 to 40, 70 to 83, and 87 to 94 of SEQ ID NO: 1, and
a pharmaceutically acceptable carrier.

A fifteenth aspect of the invention relates to a pharmaceutical composition comprising a viral vector comprising the nucleic acid sequence set forth in SEQ ID NO: 43 and a pharmaceutically acceptable carrier.

TABLE 1
Table of select sequences of the invention

| SEQ | | |
|---|---|---|
| ID | | |
| NO: | Description | Sequence |
| 1 | 99 | AGAAGCCAATTAGGCCCTCAGTTTCTGCAGCGGGGATTAATAT |
| | nucleotide | GATTATGAACACCCCCAATCTCCCAGATGCTGATTCAGCCAGG |
| | long human | AGCTTAGGAGGGG |
| | RHO | |
| | promoter | |
| | fragment | |
| 2 | 350 | AAACCAGAAAGTCTCTAGCTGTCCAGAGGACATAGCACAGAGG |
| | nucleotide | CCCATGGTCCCTATTTCAAACCCAGGCCACCAGACTGAGCTGG |
| | long human | GACCTTGGGACAGACAAGTCATGCAGAAGTTAGGGGACCTTCT |
| | RHO | CCTCCCTTTTCCTGGATCCTGAGTACCTCTCCTCCCTGACCTCAG |
| | promoter | GCTTCCTCCTAGTGTCACCTTGGCCCCTCTTAGAAGCCAATTAG |
| | fragment | GCCCTCAGTTTCTGCAGCGGGGATTAATATGATTATGAACACCC |
| | | CCAATCTCCCAGATGCTGATTCAGCCAGGAGCTTAGGAGGGGG |
| | | AGGTCACTTTATAAGGGTCTGGGGGGGTCAGAACCCAGAGTCA |
| | | TC |
| 3 | Sequence of | MLGWVQRVLPQPPGTPRKTKMQEEEEVEPEPEMEAEVEPEPNPEE |
| | the human | AETESESMPPEESFKEEEVAVADPSPQETKEAALTSTISLRAQGAEI |
| | CNGB1 | SEMNSPSHRVLTWLMKGVEKVIPQPVHSITEDPAQILGHGSTGDT |
| | protein | GCTDEPNEALEAQDTRPGLRLLLWLEQNLERVLPQPPKSSEVWRD |
| | | EPAVATAPPGRPQEMGPKLQARETPSLPTPIPLQPKEEPKEAPAPEP |
| | | QPGSQAQTSSLPPTRDPARLVAWVLHRLEMALPQPVLHGKIGEQE |
| | | PDSPGICDVQTISILPGGQVEPDLVLEEVEPPWEDAHQDVSTSPQGT |
| | | EVVPAYEEENKAVEKMPRELSRIEEEKEDEEEEEEEEEEEEEEEVT |
| | | EVLLDSCVVSQVGVGQSEEDGTRPQSTSDQKLWEEVGEEAKKEA |
| | | EEKAKEEAEEVAEEEAEKEPQDWAETKEEPEAEAEAASSGVPATK |
| | | QHPEVQVEDTDADSCPLMAEENPPSTVLPPPSPAKSDTLIVPSSASG |
| | | THRKKLPSEDDEAEELKALSPAESPVVAWSDPTTPKDTDGQDRAA |
| | | STASTNSAIINDRLQELVKLFKERTEKVKEKLIDPDVTSDEESPKPS |
| | | PAKKAPEPAPDTKPAEAEPVEEEHYCDMLCCKFKHRPWKKYQFP |
| | | QSIDPLTNLMYVLWLFFVVMAWNWNCWLIPVRWAFPYQTPDNIH |
| | | HWLLMDYLCDLIYFLDITVFQTRLQFVRGGDIITDKKDMRNNYLK |
| | | SRRFKMDLLSLLPLDFLYLKVGVNPLLRLPRCLKYMAFFEFNSRLE |
| | | SILSKAYVYRVIRTTAYLLYSLHLNSCLYYWASAYQGLGSTHWVY |
| | | DGVGNSYIRCYYFAVKTLITIGGLPDPKTLFEIVFQLLNYFTGVFAF |
| | | SVMIGQMRDVVGAATAGQTYYRSCMDSTVKYMNFYKIPKSVQN |
| | | RVKTWYEYTWHSQGMLDESELMVQLPDKMRLDLAIDVNYNIVS |
| | | KVALFQGCDRQMIFDMLKRLRSVVYLPNDYVCKKGEIGREMYIIQ |
| | | AGQVQVLGGPDGKSVLVTLKAGSVFGEISLLAVGGGNRRTANVV |
| | | AHGFTNLFILDKKDLNEILVHYPESQKLLRKKARRMLRSNNKPKE |
| | | EKSVLILPPRAGTPKLFNAALAMTGKMGGKGAKGGKLAHLRARL |
| | | KELAALEAAAKQQELVEQAKSSQDVKGEEGSAAPDQHTHPKEAA |
| | | TDPPAPRTPPEPPGSPPSSPPPASLGRPEGEEEGPAEPEEHSVRICMS |
| | | PGPEPGEQILSVKMPEEREEKAE |
| 4 | SV40 | GGCCGCAGACATGATAAGATACATTGATGAGTTTGGACAAACC |
| | polyadenyl- | ACAACTAGAATGCAGTGAAAAAAATGCTTTATTTGTGAAATTT |
| | ation signal | GTGATGCTATTGCTTTATTTGTAACCATTATAAGCTGCAATAAA |
| | | CAAGTTAACAACAACAATTGCATTCATTTTATGTTTCAGGTTCA |
| | | GGGGGAGGTGTGGGAGGTTTTTTAAAGCAAGTAAAACCTCTAC |
| | | AAATGTGGTA |
| 5 | Left | CTGCGCGCTCGCTCGCTCACTGAGGCCGCCCGGGCAAAGCCCG |
| | inverted | GGCGTCGGGCGACCTTTGGTCGCCCGGCCTCAGTGAGCGAGCG |
| | terminal | AGCGCGCAGAGAGGGAGTGGCCAACTCCATCACTAGGGGTTCC |
| | repeat (L- | T |
| | ITR) | |
| 6 | Right | AGGAACCCCTAGTGATGGAGTTGGCCACTCCCTCTCTGCGCGCT |
| | inverted | CGCTCGCTCACTGAGGCCGGGCGACCAAAGGTCGCCCGACGCC |
| | terminal | CGGGCTTTGCCCGGGCGGCCTCAGTGAGCGAGCGAGCGCGCAG |
| | repeat (R- | C |
| | ITR) | |
| 7 | Sequence of | CAGCTGCGCGCTCGCTCGCTCACTGAGGCCGCCCGGGCAAAGC |
| | vector | CCGGGCGTCGGGCGACCTTTGGTCGCCCGGCCTCAGTGAGCGA |
| | construct: | GCGAGCGCGCAGAGAGGGAGTGGCCAACTCCATCACTAGGGGT |
| | pGL2.0- | TCCTTGTAGTTAATGATTAACCCGCCATGCTACTTATCTACGTA |
| | hRho194- | GCCATGCTCTAGGAAGATCGGAATTCGCCCTTAAGCCTCTCCTC |
| | hCNGB1a- | CCTGACCTCAGGCTTCCTCCTAGTGTCACCTTGGCCCCTCTTAG |
| | SV40 | AAGCCAATTAGGCCCTCAGTTTCTGCAGCGGGGATTAATATGA |
| | | TTATGAACACCCCCAATCTCCCAGATGCTGATTCAGCCAGGAG |
| | | CTTAGGAGGGGGAGGTCACTTTATAAGGGTCTGGGGGGGTCAG |
| | | AACCCAGAGTCATCACTAGTAACGGCCGCCAGTGTGCTGGAAT |
| | | TCGCCCTTCTCCACCGCCATGTTGGGCTGGGTCCAGAGGGTGCT |
| | | GCCTCAGCCCCCAGGGACCCCTCGGAAGACCAAGATGCAGGAG |
| | | GAAGAGGAAGTGGAACCAGAGCCAGAGATGGAGGCGGAGGTG |
| | | GAACCAGAACCGAATCCTGAGGAGGCCGAGACAGAGTCCGAG |
| | | TCCATGCCCCCCGAAGAGTCATTCAAGGAGGAGGAAGTGGCTG |
| | | TGGCAGACCCAAGCCCTCAGGAGACCAAGGAGGCTGCCCTTAC |
| | | TTCCACCATATCCCTCCGGGCCCAGGGCGCTGAGATTTCTGAAA |
| | | TGAATAGTCCCAGCCACAGGGTACTGACCTGGCTCATGAAGGG |
| | | TGTAGAGAAGGTGATCCCGCAGCCTGTTCACAGCATCACGGAG |
| | | GACCCGGCTCAGATCCTGGGGCATGGCAGCACTGGGGACACAG |
| | | GGTGCACAGATGAACCCAATGAGGCCCTTGAGGCCCAAGACAC |
| | | TAGGCCTGGGCTGCGGCTGCTTCTGTGGCTGGAGCAGAATCTG |
| | | GAAAGAGTGCTTCCTCAGCCCCCCAAATCCTCTGAGGTCTGGA |
| | | GAGATGAGCCTGCAGTTGCTACAGCGCCTCCAGGACGCCCCCA |
| | | GGAAATGGGGCCCAAGCTGCAGGCCCGGGAGACCCCCTCCCTG |
| | | CCCACACCCATCCCCCTGCAGCCCAAGGAGGAACCCAAGGAGG |
| | | CACCAGCTCCAGAGCCCCAGCCCGGCTCCCAGGCCCAGACCTC |
| | | CTCCCTGCCACCAACCAGGGACCCTGCCAGGCTGGTGGCATGG |
| | | GTCCTGCACAGGCTGGAGATGGCCTTGCCGCAGCCAGTGCTAC |
| | | ATGGGAAAATAGGGGAACAGGAGCCTGACTCCCCTGGGATATG |
| | | TGATGTGCAGACCATCAGCATCCTTCCTGGAGGACAAGTGGAG |
| | | CCTGACCTTGTCCTAGAGGAGGTTGAACCGCCCTGGGAGGATG |
| | | CCCACCAGGATGTCAGTACCAGCCCACAGGGTACAGAGGTGGT |
| | | TCCAGCTTATGAAGAAGAGAACAAAGCTGTGGAGAAGATGCCC |
| | | AGAGAGCTGTCCCGGATTGAAGAGGAGAAAGAAGATGAGGAG |
| | | GAGGAAGAGGAAGAGGAGGAGGAGGAGGAAGAGGAGGAGGT |
| | | GACTGAGGTGCTGCTGGATAGCTGTGTGGTGTCGCAGGTGGGC |
| | | GTGGGCCAGAGTGAAGAAGACGGGACCCGGCCCCAGAGCACT |
| | | TCAGATCAGAAGCTGTGGGAGGAAGTTGGGGAGGAGGCCAAG |
| | | AAGGAGGCTGAAGAGAAGGCCAAGGAGGAGGCCGAGGAGGTG |
| | | GCTGAAGAGGAGGCTGAAAAGGAGCCCCAGGACTGGGCGGAG |
| | | ACCAAGGAGGAGCCTGAGGCTGAGGCCGAGGCTGCCAGTTCAG |
| | | GAGTGCCTGCCACGAAACAGCACCCAGAAGTGCAGGTGGAAG |
| | | ATACTGATGCTGATAGCTGCCCCCTCATGGCAGAAGAGAATCC |
| | | ACCCTCAACCGTGTTGCCGCCACCATCTCCTGCCAAATCAGACA |
| | | CCCTTATAGTCCCAAGCTCAGCCTCGGGGACACACAGGAAGAA |
| | | GCTGCCCTCTGAGGATGATGAGGCTGAAGAGCTCAAGGCGTTG |
| | | TCACCAGCAGAGTCCCCAGTGGTTGCCTGGTCTGACCCCACCAC |
| | | CCCGAAGGACACTGATGGCCAGGACCGTGCGGCCTCCACGGCC |
| | | AGCACAAATAGCGCCATCATCAACGACCGGCTCCAGGAGCTGG |
| | | TGAAGCTCTTCAAGGAGCGGACAGAGAAAGTGAAGGAGAAAC |
| | | TCATTGACCCTGACGTCACCTCTGATGAGGAGAGCCCCAAGCC |
| | | CTCCCCAGCCAAGAAAGCCCCAGAGCCAGCTCCAGACACAAAG |
| | | CCCGCTGAAGCCGAGCCAGTGGAAGAGGAGCACTATTGCGACA |
| | | TGCTCTGCTGCAAGTTCAAACACCGCCCCTGGAAGAAGTACCA |
| | | GTTTCCCCAGAGCATTGACCCGCTGACCAACCTGATGTATGTCC |
| | | TATGGCTGTTCTTCGTGGTGATGGCCTGGAATTGGAACTGTTGG |
| | | CTGATTCCCGTGCGCTGGGCCTTCCCCTACCAGACCCCGGACAA |
| | | CATCCACCACTGGCTGCTGATGGATTACCTATGCGACCTCATCT |
| | | ACTTCCTGGACATCACCGTGTTCCAGACACGCCTGCAGTTTGTC |
| | | AGAGGCGGGGACATCATTACGGACAAAAAGGACATGCGAAAT |
| | | AACTACCTGAAGTCTCGCCGCTTCAAGATGGACCTGCTCAGCCT |
| | | CCTGCCCTTGGATTTTCTCTATTTGAAAGTCGGTGTGAACCCCC |
| | | TCCTCCGCCTGCCCCGCTGTTTAAAGTACATGGCCTTCTTCGAG |
| | | TTTAACAGCCGCCTGGAATCCATCCTCAGCAAAGCCTACGTGTA |
| | | CAGGGTCATCAGGACCACAGCCTACCTTCTCTACAGCCTGCATT |
| | | TGAATTCCTGTCTTTATTACTGGGCATCGGCCTATCAGGGCCTC |
| | | GGCTCCACTCACTGGGTTTACGATGGCGTGGGAAACAGTTATA |
| | | TTCGCTGTTACTACTTTGCTGTGAAGACCCTCATCACCATCGGG |
| | | GGGCTGCCTGACCCCAAGACACTCTTTGAAATTGTCTTCCAGCT |
| | | GCTGAATTATTTCACGGGCGTCTTTGCTTTCTCTGTGATGATCG |
| | | GACAGATGAGAGATGTGGTAGGGGCCGCCACCGCGGGACAGA |
| | | CCTACTACCGCAGCTGCATGGACAGCACGGTGAAGTACATGAA |
| | | TTTCTACAAGATCCCCAAGTCCGTGCAGAACCGCGTCAAGACC |
| | | TGGTACGAGTACACCTGGCACTCGCAAGGCATGCTGGATGAGT |
| | | CAGAGCTGATGGTGCAGCTTCCAGACAAGATGCGGCTGGACCT |
| | | CGCCATCGACGTGAACTACAACATCGTTAGCAAAGTCGCACTC |
| | | TTTCAGGGCTGTGACCGGCAGATGATCTTTGACATGCTGAAGA |
| | | GGCTTCGCTCTGTTGTCTACCTGCCCAACGACTATGTGTGCAAG |
| | | AAGGGGGAGATCGGCCGTGAGATGTACATCATCCAGGCAGGGC |
| | | AAGTGCAGGTCTTGGGCGGCCCTGATGGGAAATCTGTGCTGGT |
| | | GACGCTGAAAGCTGGATCTGTGTTTGGAGAAATAAGCTTGCTG |
| | | GCTGTTGGGGGCGGGAACCGGCGCACGGCCAACGTGGTGGCGC |
| | | ACGGGTTTACCAACCTCTTCATCCTGGATAAGAAGGACCTGAA |
| | | TGAGATTTTGGTGCATTATCCTGAGTCTCAGAAGTTACTCCGGA |
| | | AGAAAGCCAGGCGCATGCTGAGAAGCAACAATAAGCCCAAGG |
| | | AGGAGAAGAGCGTGCTGATCCTTCCACCCCGGGCGGGCACCCC |
| | | AAAGCTCTTCAACGCTGCCCTCGCTATGACAGGAAAGATGGGT |
| | | GGCAAGGGGGCAAAAGGCGGCAAACTTGCTCACCTCCGGGCCC |
| | | GGCTCAAAGAACTGGCCGCGCTGGAGGCGGCTGCAAAGCAGC |
| | | AAGAGTTGGTGGAACAGGCCAAGAGCTCGCAAGACGTCAAGG |
| | | GAGAGGAAGGCTCCGCCGCCCCAGACCAGCACACGCACCCAA |
| | | AGGAGGCCGCCACCGACCCACCCGCGCCCCGGACGCCCCCCGA |
| | | GCCCCCGGGGTCTCCACCGAGCTCTCCACCGCCTGCCTCCCTTG |
| | | GGAGGCCGGAGGGAGAGGAGGAGGGGCCGGCCGAGCCCGAAG |
| | | AGCACTCGGTGAGGATCTGCATGAGCCCGGGCCCGGAGCCGGG |
| | | AGAGCAGATCCTGTCGGTGAAGATGCCGGAGGAAAGGGAGGA |
| | | GAAGGCGGAGTAAGGTGGGGTGAGGCGGATCCATGGCCGCAG |
| | | ACATGATAAGATACATTGATGAGTTTGGACAAACCACAACTAG |
| | | AATGCAGTGAAAAAAATGCTTTATTTGTGAAATTTGTGATGCTA |
| | | TTGCTTTATTTGTAACCATTATAAGCTGCAATAAACAAGTTAAC |
| | | AACAACAATTGCATTCATTTTATGTTTCAGGTTCAGGGGGAGGT |
| | | GTGGGAGGTTTTTTAAAGCAAGTAAAACCTCTACAAATGTGGT |
| | | ACTCGAGTTAAGGGCGAATTCCCGATAAGGATCTTCCTAGAGC |
| | | ATGGCTACGTAGATAAGTAGCATGGCGGGTTAATCATTAACTA |
| | | CAAGGAACCCCTAGTGATGGAGTTGGCCACTCCCTCTCTGCGC |
| | | GCTCGCTCGCTCACTGAGGCCGGGCGACCAAAGGTCGCCCGAC |
| | | GCCCGGGCTTTGCCCGGGCGGCCTCAGTGAGCGAGCGAGCGCG |
| | | CAGCTGGGCCTCAGTGAGCGAGCGAGCGCGCAGCTGCATTAAT |
| | | GAATCGGCCAACGCGCGGGGAGAGGCGGTTTGCGTATTGGGCG |
| | | CTCTTCCGCTTCCTCGCTCACTGACTCGCTGCGCTCGGTCGTTCG |
| | | GCTGCGGCGAGCGGTATCAGCTCACTCAAAGGCGGTAATACGG |
| | | TTATCCACAGAATCAGGGGATAACGCAGGAAAGAACATGTCGC |
| | | GTTGCTGGCGTTTTTCCATAGGCTCCGCCCCCCTGACGAGCATC |
| | | ACAAAAATCGACGCTCAAGTCAGAGGTGGCGAAACCCGACAG |
| | | GACTATAAAGATACCAGGCGTTTCCCCCTGGAAGCTCCCTCGTG |
| | | CGCTCTCCTGTTCCGACCCTGCCGCTTACCGGATACCTGTCCGC |
| | | CTTTCTCCCTTCGGGAAGCGTGGCGCTTTCTCATAGCTCACGCT |
| | | GTAGGTATCTCAGTTCGGTGTAGGTCGTTCGCTCCAAGCTGGGC |
| | | TGTGTGCACGAACCCCCCGTTCAGCCCGACCGCTGCGCCTTATC |
| | | CGGTAACTATCGTCTTGAGTCCAACCCGGTAAGACACGACTTAT |
| | | CGCCACTGGCAGCAGCCACTGGTAACAGGATTAGCAGAGCGAG |
| | | GTATGTAGGCGGTGCTACAGAGTTCTTGAAGTGGTGGCCTAAC |
| | | TACGGCTACACTAGAAGAACAGTATTTGGTATCTGCGCTCTGCT |
| | | GAAGCCAGTTACCTTCGGAAAAAGAGTTGGTAGCTCTTGATCC |
| | | GGCAAACAAACCACCGCTGGTAGCGGTGGTTTTTTTGTTTGCAA |
| | | GCAGCAGATTACGCGCAGAAAAAAAGGATCTCAAGAAGATCCT |
| | | TTGATCTTTTCTACGGGGTCTGACGCTCAGTGGAACGAAAACTC |
| | | ACGTTAAGGGATTTTGGTCATGACTGTGGAATGTGTGTCAGTTA |
| | | GGCGACATAGGTGATCTATGTAGAAGCCTAGTGGAACAGGTTA |
| | | GTTTGAGTAGCTTTAGAATGTAAATTCTGGGATCATAGTGTAGT |
| | | AATCTCTAATTAACGGTGACGGTTTGTAAGACAGGTCTTCGCAA |
| | | AATCAAGCGGCAGGTGATTTCAACAGATTCTTGCTGATGGTTTA |
| | | GGCGTACAATGCCCTGAAGAATAAGTAAGAGAATAGCACTCCT |
| | | CGTCGCCTAGAATTACCTACCGGCGTCCACCATACCTTCGATTA |
| | | TCGCGCCCACTCTCCCATTAGTCGGCACAGGTGGATGTGTTGCG |
| | | ATAGCCCGCTAAGATATTCTAAGGCGTAACGCAGATGAATATT |
| | | CTACAGAGTTGCCATAGGCGTTGAACGCTTCACGGACGATAGG |
| | | AATGTTGCGTATAGAGCGTGAGTCATCGAAGTGGTGTATACAC |
| | | TCGTACTTAACATCTAGCCCGGCTCTATCAGTACACCAGTGCCT |
| | | TGAATGACATACTCATCATTAAACTTTCTCAACAGTCAAACGAC |
| | | CAAGTGCATTTCCAAGGAGTGCGAAGGAGATTCATTCTCTCGC |
| | | CAGCACTGTAATAGGCACTAAAAGAGTGAAGATAAGCTAGAGT |
| | | GCCGTGCTAAGACGGTGTCGGAACAAAGCGGTCTTACGGTCAG |
| | | TCGTATTTCCTGTCGAGTCCCGTCCAGTTGAGCGTATCACTCCC |
| | | AGTGTACTAGCAAGCCGAGAAGGCTGTGCTTGGAGTCAATCGG |
| | | ATGTAGGATGGTCTCCAGACACCGGGCCACCACTCTTCACGCCT |
| | | AGAAGCATAGAACGTCGAGCAGACATCAAAGTCTTAGTACCGG |
| | | ACGTGCCGTTTCACTGCGAATATTACCTGAAGCTGTACCGTTAT |
| | | TGCGGAGCAAAGTGACAGTGCTGCTCTTATCATATTTGTATTGA |
| | | CGACAGCCGCCTTCGCGGTTTCCTCAGACTCTAGATCGAATACA |
| | | GGCTTATTGTAGGCAGAGGCACGCCCTTGTTAGTGGCTGCGGC |
| | | AATATCTTCCGATCCCCTTGTCTAACCATGAATCAATTCTCTCA |
| | | TTTGAAGACCCTAATATGTCATCATTAGTGTTTCAAATGCCACC |
| | | AAATACCGCCTAGAAATGTCTATGATGTGTGTCCACTAGAAGTT |
| | | GATTCACAAACGACTGCTAGAATCGCGTGATAGGGCATCTTGA |
| | | AGTTTACATTGTTGTATCGCAAGGTACTCCGATCTTAATGGATG |
| | | CGAAGTGGTACGGATGCAATCAAGCGCGTGAGAGCGGTACATT |
| | | AGAGCGTTCACCTACGCTACGCTAACGGGCGATTCTGATAAGA |
| | | ATGCACATTGCGTCGATTCATAAGATGTCTCGACCGCATGCGCA |
| | | ACTTGTGAAGTGTCTACTATCCCTAAGCGCATATCTCGCACAGT |
| | | AACCGAATATGTCGGCATCTGATGTTACCGTTGAGTTAGTGTTC |
| | | AGCTCACGGAACTTATTGTATGAGTAGAGATTTGTAAGAGCTG |
| | | TTAGTTAGCTCGCTCAGCTAATAGTTGCCCACACAACGTCAAAT |
| | | TAGAGAACGGTCGTAACATTATCGGTGGTTCTCTAACTACTATC |
| | | AGTACCCACGACTCGACTCTGCCGCAGCTAGGTATCGCCTGAA |
| | | AGCCAGTCAGCGTTAAGGAGTGCTCTGACCAGGACAACAGGCG |
| | | TAGTGAGAGTTACTTGTTCGTTGCTCTTCCGACTCGGACCTGAG |
| | | TTCGCCAACGACCCACTTGAGGTCTGAGCCGGTGAAGAGAAGT |
| | | AAGCATCTCGTTCGCAGCTTGCCAGCACTTTCAGAACATGACCC |
| | | CTATTTGTTTATTTTTCTAAATACATTCAAATATGTATCCGCTCA |
| | | TGAGACAATAACCCTGATAAATGCTTCAATAATATTGAAAAAG |
| | | GAAGAGTGGCCGCCTCGGCCTAGGCTTTTGCAAAGATCGATCA |
| | | AGAGACAGGATGAGGATCGTTTCGCATGATTGAACAAGATGGA |
| | | TTGCACGCAGGTTCTCCGGCCGCTTGGGTGGAGAGGCTATTCG |
| | | GCTATGACTGGGCACAACAGACAATCGGCTGCTCTGATGCCGC |
| | | CGTGTTCCGGCTGTCAGCGCAGGGGCGCCCGGTTCTTTTTGTCA |
| | | AGACCGACCTGTCCGGTGCCCTGAATGAACTGCAAGACGAGGC |
| | | AGCGCGGCTATCGTGGCTGGCCACGACGGGCGTTCCTTGCGCA |
| | | GCTGTGCTCGACGTTGTCACTGAAGCGGGAAGGGACTGGCTGC |
| | | TATTGGGCGAAGTGCCGGGGCAGGATCTCCTGTCATCTCACCTT |
| | | GCTCCTGCCGAGAAAGTATCCATCATGGCTGATGCAATGCGGC |
| | | GGCTGCATACGCTTGATCCGGCTACCTGCCCATTCGACCACCAA |
| | | GCGAAACATCGCATCGAGCGAGCACGTACTCGGATGGAAGCCG |
| | | GTCTTGTCGATCAGGATGATCTGGACGAAGAGCATCAGGGGCT |
| | | CGCGCCAGCCGAACTGTTCGCCAGGCTCAAGGCGAGCATGCCC |
| | | GACGGCGAGGATCTCGTCGTGACCCATGGCGATGCCTGCTTGC |
| | | CGAATATCATGGTGGAAAATGGCCGCTTTTCTGGATTCATCGAC |
| | | TGTGGCCGGCTGGGTGTGGCGGACCGCTATCAGGACATAGCGT |
| | | TGGCTACCCGTGATATTGCTGAAGAGCTTGGCGGCGAATGGGC |
| | | TGACCGCTTCCTCGTGCTTTACGGTATCGCCGCTCCCGATTCGC |
| | | AGCGCATCGCCTTCTATCGCCTTCTTGACGAGTTCTTCTGAGGT |
| | | ACCATGATGCGTGCATGGTAGAATGACTCTTGATAACGGACTT |
| | | CGACTAGGCAATATCCCTTGTCAACTTGTCGAGGAGAAAAGTA |
| | | TTGACTGAAGCGCTCCCGGCACAACGGCCAAAGAAGTCTCAGC |
| | | AATGTTCTTATTTCCGAATGACATGCGTCTCCTTGCGGGTAAAT |
| | | CGCCGACCGCAAAACTTAGGAGCCAGGATACAGATAGGTCTAA |
| | | CTTAGGTTAAGGGAGTAAATCCTGGGATCGTTCAGTTGTAACC |
| | | ATATACTTACGCTGGGGCTTCTCCGGCGGATGTTACTGTCACCA |
| | | ACCACGAGATTTGAAGTAAACGCATGATTGAGCACATAGCCGC |
| | | GCTATCCGACAATCTCCAAATTGATAACATACCGTTCCATGAAG |
| | | GCCAGAATTACTTACCGGCCCTTTCCATGCGTGCGCCATACCGC |
| | | ACTCTGCGCTTATCCGTCCGAGGGGAGAGTGTGCGATCCTCCGT |
| | | TAAGATATTCTCACGTATGACGTAGCTATGTATTGTGCAGAGGT |
| | | AGCGAAGGCGTTGAACACTTCACAGATGGTGGGGATTCGGGCA |
| | | AAGGGCGTGATAACTTGGGGACTAACATAGGCGTAAACTACGA |
| | | TGGCACCAACTCAATCGCAGCTCGTGCGCCCTGAATCAACGTA |
| | | CTCATCTCAACTGATTCTCGGCAATCTACGGAGCGACTTGATTA |
| | | TCAACACCTGTCTAGCAGTTCTAATCTTCTGCCAACATCGTACA |
| | | TAGCCTCCAAGAGATTATCATACCTATCGGCACAGAAGTGACA |
| | | CGACGCCGAAGGGTAGCGGACTTCTGGTCAACCACAATTCCCC |
| | | AGGGGACAGGTCCTGCGGTGCGCATCACTTTGTAAGTGCAAGC |
| | | AACCCAAGTGAGCCCAGCCTGGACTGAGCTGGTTCCTGTGTCA |
| | | GGTCGAGGCTGGGGATGACAGCTCTTGTAAACATAGTGATCAA |
| | | GCGTGGCGTCGAACGGTCGAGAAACTCATAGTACCTCGGGTAG |
| | | CAACTTACTCAGGTTATTGCTTGAAGCTGTACTATTTCAGGAGC |
| | | GCTGAAGGTCTCTTCTTCTGTAGACTGAACTCGCAAGGGTCGTG |
| | | AAGTCGGTTCCTTCAATGCTTAACAAGAACAAAGGCTTACTGT |
| | | GCAGACTGGAACGCCCATCTAGCGGCTCGCGTCTTGAATGCTC |
| | | GGTCCCCTTTGTCATTGCGGATACAATCCATTTCCCTCATTCAC |
| | | CAGCTTGCGAAGTCTACATTGAGTAGACGAATGCGACCTAGAA |
| | | GAGGTGCGCTTCAGAACTTGTGAGGAGTGGTTGATGCTCTATA |
| | | CTCCATTTGGTGTTTCGTGCATCACCGCGATAGGCTGACAAGAG |
| | | GTCTTGAACATTGAATAGCAAGGCACTTCCGGTCTCATAGAAG |
| | | AGAGCACGGGATAAGGTACGCGCGTGGTACGGGAGGATCAAG |
| | | GGGCTACACGATAGAAAGGCTTCTCCCTCACTCGCTAGGAGGC |
| | | AAATGCAGAACGCTGGTTACTACTACGATACGTGAAACTTGTC |
| | | CAACGGTTGCCCAAAGTGTTAAGTGTCTATCACCCTAGTGCCGT |
| | | TTCCCGGAGAAAACGCCAGGTTGAATCCGCATTTGAAGCTACG |
| | | ATGGTGAAGTCTGGGTCGAGCGCGCCGCATGTTGATTGCGTGA |
| | | GTAGGCTCGACCAAGAACCGCTAGTAGCGTCGCTGTAGAAATA |
| | | GTTCTCGACAGACCGTCGAGTTTAGAAAATGGTAGCAGCATTG |
| | | TTCGCATCTCAATCAAGTATGGATTACGGTGTTTACACTGTCCT |
| | | GCGGCTACCCATCGCCTGAAATCCAGCTCGTGTCAAGCCATTGC |
| | | CTCTCCGGGACGCCGCATGAAGTAACTACATATACCTTGCACG |
| | | GGTTGACTGCGGTCCGTTCAGACTCGACCAAGGACACAATCCA |
| | | GCGATCGGTGCGGGCCTCTTCGCTATTACGC |
| 8 | Sequence of | ATGTTGGGCTGGGTCCAGAGGGTGCTGCCTCAGCCCCCAGGGA |
| | the human | CCCCTCGGAAGACCAAGATGCAGGAGGAAGAGGAAGTGGAAC |
| | CNGB1 | CAGAGCCAGAGATGGAGGCGGAGGTGGAACCAGAACCGAATC |
| | gene | CTGAGGAGGCCGAGACAGAGTCCGAGTCCATGCCCCCCGAAGA |
| | | GTCATTCAAGGAGGAGGAAGTGGCTGTGGCAGACCCAAGCCCT |
| | | CAGGAGACCAAGGAGGCTGCCCTTACTTCCACCATATCCCTCC |
| | | GGGCCCAGGGCGCTGAGATTTCTGAAATGAATAGTCCCAGCCA |
| | | CAGGGTACTGACCTGGCTCATGAAGGGTGTAGAGAAGGTGATC |
| | | CCGCAGCCTGTTCACAGCATCACGGAGGACCCGGCTCAGATCC |
| | | TGGGGCATGGCAGCACTGGGGACACAGGGTGCACAGATGAAC |
| | | CCAATGAGGCCCTTGAGGCCCAAGACACTAGGCCTGGGCTGCG |
| | | GCTGCTTCTGTGGCTGGAGCAGAATCTGGAAAGAGTGCTTCCTC |
| | | AGCCCCCCAAATCCTCTGAGGTCTGGAGAGATGAGCCTGCAGT |
| | | TGCTACAGCGCCTCCAGGACGCCCCCAGGAAATGGGGCCCAAG |
| | | CTGCAGGCCCGGGAGACCCCCTCCCTGCCCACACCCATCCCCCT |
| | | GCAGCCCAAGGAGGAACCCAAGGAGGCACCAGCTCCAGAGCC |
| | | CCAGCCCGGCTCCCAGGCCCAGACCTCCTCCCTGCCACCAACC |
| | | AGGGACCCTGCCAGGCTGGTGGCATGGGTCCTGCACAGGCTGG |
| | | AGATGGCCTTGCCGCAGCCAGTGCTACATGGGAAAATAGGGGA |
| | | ACAGGAGCCTGACTCCCCTGGGATATGTGATGTGCAGACCATC |
| | | AGCATCCTTCCTGGAGGACAAGTGGAGCCTGACCTTGTCCTAG |
| | | AGGAGGTTGAACCGCCCTGGGAGGATGCCCACCAGGATGTCAG |
| | | TACCAGCCCACAGGGTACAGAGGTGGTTCCAGCTTATGAAGAA |
| | | GAGAACAAAGCTGTGGAGAAGATGCCCAGAGAGCTGTCCCGG |
| | | ATTGAAGAGGAGAAAGAAGATGAGGAGGAGGAAGAGGAAGA |
| | | GGAGGAGGAGGAGGAAGAGGAGGAGGTGACTGAGGTGCTGCT |
| | | GGATAGCTGTGTGGTGTCGCAGGTGGGCGTGGGCCAGAGTGAA |
| | | GAAGACGGGACCCGGCCCCAGAGCACTTCAGATCAGAAGCTGT |
| | | GGGAGGAAGTTGGGGAGGAGGCCAAGAAGGAGGCTGAAGAGA |
| | | AGGCCAAGGAGGAGGCCGAGGAGGTGGCTGAAGAGGAGGCTG |
| | | AAAAGGAGCCCCAGGACTGGGCGGAGACCAAGGAGGAGCCTG |
| | | AGGCTGAGGCCGAGGCTGCCAGTTCAGGAGTGCCTGCCACGAA |
| | | ACAGCACCCAGAAGTGCAGGTGGAAGATACTGATGCTGATAGC |
| | | TGCCCCCTCATGGCAGAAGAGAATCCACCCTCAACCGTGTTGC |
| | | CGCCACCATCTCCTGCCAAATCAGACACCCTTATAGTCCCAAGC |
| | | TCAGCCTCGGGGACACACAGGAAGAAGCTGCCCTCTGAGGATG |
| | | ATGAGGCTGAAGAGCTCAAGGCGTTGTCACCAGCAGAGTCCCC |
| | | AGTGGTTGCCTGGTCTGACCCCACCACCCCGAAGGACACTGAT |
| | | GGCCAGGACCGTGCGGCCTCCACGGCCAGCACAAATAGCGCCA |
| | | TCATCAACGACCGGCTCCAGGAGCTGGTGAAGCTCTTCAAGGA |
| | | GCGGACAGAGAAAGTGAAGGAGAAACTCATTGACCCTGACGTC |
| | | ACCTCTGATGAGGAGAGCCCCAAGCCCTCCCCAGCCAAGAAAG |
| | | CCCCAGAGCCAGCTCCAGACACAAAGCCCGCTGAAGCCGAGCC |
| | | AGTGGAAGAGGAGCACTATTGCGACATGCTCTGCTGCAAGTTC |
| | | AAACACCGCCCCTGGAAGAAGTACCAGTTTCCCCAGAGCATTG |
| | | ACCCGCTGACCAACCTGATGTATGTCCTATGGCTGTTCTTCGTG |
| | | GTGATGGCCTGGAATTGGAACTGTTGGCTGATTCCCGTGCGCTG |
| | | GGCCTTCCCCTACCAGACCCCGGACAACATCCACCACTGGCTG |
| | | CTGATGGATTACCTATGCGACCTCATCTACTTCCTGGACATCAC |
| | | CGTGTTCCAGACACGCCTGCAGTTTGTCAGAGGCGGGGACATC |
| | | ATTACGGACAAAAAGGACATGCGAAATAACTACCTGAAGTCTC |
| | | GCCGCTTCAAGATGGACCTGCTCAGCCTCCTGCCCTTGGATTTT |
| | | CTCTATTTGAAAGTCGGTGTGAACCCCCTCCTCCGCCTGCCCCG |
| | | CTGTTTAAAGTACATGGCCTTCTTCGAGTTTAACAGCCGCCTGG |
| | | AATCCATCCTCAGCAAAGCCTACGTGTACAGGGTCATCAGGAC |
| | | CACAGCCTACCTTCTCTACAGCCTGCATTTGAATTCCTGTCTTTA |
| | | TTACTGGGCATCGGCCTATCAGGGCCTCGGCTCCACTCACTGGG |
| | | TTTACGATGGCGTGGGAAACAGTTATATTCGCTGTTACTACTTT |
| | | GCTGTGAAGACCCTCATCACCATCGGGGGGCTGCCTGACCCCA |
| | | AGACACTCTTTGAAATTGTCTTCCAGCTGCTGAATTATTTCACG |
| | | GGCGTCTTTGCTTTCTCTGTGATGATCGGACAGATGAGAGATGT |
| | | GGTAGGGGCCGCCACCGCGGGACAGACCTACTACCGCAGCTGC |
| | | ATGGACAGCACGGTGAAGTACATGAATTTCTACAAGATCCCCA |
| | | AGTCCGTGCAGAACCGCGTCAAGACCTGGTACGAGTACACCTG |
| | | GCACTCGCAAGGCATGCTGGATGAGTCAGAGCTGATGGTGCAG |
| | | CTTCCAGACAAGATGCGGCTGGACCTCGCCATCGACGTGAACT |
| | | ACAACATCGTTAGCAAAGTCGCACTCTTTCAGGGCTGTGACCG |
| | | GCAGATGATCTTTGACATGCTGAAGAGGCTTCGCTCTGTTGTCT |
| | | ACCTGCCCAACGACTATGTGTGCAAGAAGGGGGAGATCGGCCG |
| | | TGAGATGTACATCATCCAGGCAGGGCAAGTGCAGGTCTTGGGC |
| | | GGCCCTGATGGGAAATCTGTGCTGGTGACGCTGAAAGCTGGAT |
| | | CTGTGTTTGGAGAAATAAGCTTGCTGGCTGTTGGGGGCGGGAA |
| | | CCGGCGCACGGCCAACGTGGTGGCGCACGGGTTTACCAACCTC |
| | | TTCATCCTGGATAAGAAGGACCTGAATGAGATTTTGGTGCATTA |
| | | TCCTGAGTCTCAGAAGTTACTCCGGAAGAAAGCCAGGCGCATG |
| | | CTGAGAAGCAACAATAAGCCCAAGGAGGAGAAGAGCGTGCTG |
| | | ATCCTTCCACCCCGGGCGGGCACCCCAAAGCTCTTCAACGCTGC |
| | | CCTCGCTATGACAGGAAAGATGGGTGGCAAGGGGGCAAAAGG |
| | | CGGCAAACTTGCTCACCTCCGGGCCCGGCTCAAAGAACTGGCC |
| | | GCGCTGGAGGCGGCTGCAAAGCAGCAAGAGTTGGTGGAACAG |
| | | GCCAAGAGCTCGCAAGACGTCAAGGGAGAGGAAGGCTCCGCC |
| | | GCCCCAGACCAGCACACGCACCCAAAGGAGGCCGCCACCGACC |
| | | CACCCGCGCCCCGGACGCCCCCCGAGCCCCCGGGGTCTCCACC |
| | | GAGCTCTCCACCGCCTGCCTCCCTTGGGAGGCCGGAGGGAGAG |
| | | GAGGAGGGGCCGGCCGAGCCCGAAGAGCACTCGGTGAGGATC |
| | | TGCATGAGCCCGGGCCCGGAGCCGGGAGAGCAGATCCTGTCGG |
| | | TGAAGATGCCGGAGGAAAGGGAGGAGAAGGCGGAGTAA |
| 9 | 194 | TCTCCTCCCTGACCTCAGGCTTCCTCCTAGTGTCACCTTGGCCCC |
| | nucleotide | TCTTAGAAGCCAATTAGGCCCTCAGTTTCTGCAGCGGGGATTAA |
| | long | TATGATTATGAACACCCCCAATCTCCCAGATGCTGATTCAGCCA |
| | fragment of | GGAGCTTAGGAGGGGGAGGTCACTTTATAAGGGTCTGGGGGGG |
| | human RHO | TCAGAACCCAGAGTCATC |
| | promoter | |
| 40 | Sequence of | MLGWVQRVLPQPPGTPRKTKMQEEEEVEPEPEMEAEVEPEPNPEE |
| | the human | AETESESMPPEESFKEEEVAVADPSPQETKEAALTSTISLRAQGAEI |
| | CNGB1 | SEMNSPSHRVLTWLMKGVEKVIPQPVHSITEDPAQILGHGSTGDT |
| | protein | GCTDEPNEALEAQDTRPGLRLLLWLEQNLERVLPQPPKSSEVWRD |
| | translated | EPAVATGAASDPAPPGRPQEMGPKLQARETPSLPTPIPLQPKEEPK |
| | from next | EAPAPEPQPGSQAQTSSLPPTRDPARLVAWVLHRLEMALPQPVLH |
| | generation | GKIGEQEPDSPGICDVQTISILPGGQVEPDLVLEEVEPPWEDAHQD |
| | sequencing | VSTSPQGTEVVPAYEEENKAVEKMPRELSRIEEEKEDEEEEEEEEE |
| | (NGS) | EEEEEEVTEVLLDSCVVSQVGVGQSEEDGTRPQSTSDQLWEEVGE |
| | results | EAKKEAEEKAKEEAEEVAEEEAEKEPQDWAETKEEPEAEAEAASS |
| | | GVPATKQHPEVQVEDTDADSCPLMAEENPPSTVLPPPSPAKSDTLI |
| | | VPSSASGTHRKKLPSEDDEAEELKALSPAESPVVAWSDPTTPKDTD |
| | | GQDRAASTASTNSAIINDRLQELVKLFKERTEKVKEKLIDPDVTSD |
| | | EESPKPSPAKKAPEPAPDTKPAEAEPVEEEHYCDMLCCKFKHRPW |
| | | KKYQFPQSIDPLTNLMYVLWLFFVVMAWNWNCWLIPVRWAFPY |
| | | QTPDNIHHWLLMDYLCDLIYFLDITVFQTRLQFVRGGDIITDKKDM |
| | | RNNYLKSRRFKMDLLSLLPLDFLYLKVGVNPLLRLPRCLKYMAFF |
| | | EFNSRLESILSKAYVYRVIRTTAYLLYSLHLNSCLYYWASAYQGL |
| | | GSTHWVYDGVGNSYIRCYYFAVKTLITIGGLPDPKTLFEIVFQLLN |
| | | YFTGVFAFSVMIGQMRDVVGAATAGQTYYRSCMDSTVKYMNFY |
| | | KIPKSVQNRVKTWYEYTWHSQGMLDESELMVQLPDKMRLDLAID |
| | | VNYNIVSKVALFQGCDRQMIFDMLKRLRSVVYLPNDYVCKKGEI |
| | | GREMYIIQAGQVQVLGGPDGKSVLVTLKAGSVFGEISLLAVGGGN |
| | | RRTANVVAHGFTNLFILDKKDLNEILVHYPESQKLLRKKARRMLR |
| | | SNNKPQEEKSVLILPPRAGTPKLFNAALAMTGKMGGKGAKGGKL |
| | | AHLRARLKELAALEAAAKQQELVEQAKSSQDVKGEEGSAAPDQH |
| | | THPKEAATDPPAPRTPPEPPGSPPSSPPPASLGRPEGEEEGPAEPEEH |
| | | SVRICMSPGPEPGEQILSVKMPEEREEKAE |
| 41 | Sequence of | MLGWVQRVLPQPPGTPRKTKMQEEEEVEPEPEMEAEVEPEPNPEE |
| | the CNGB1 | AETESESMPPEESFKEEEVAVADPSPQETKEAALTSTISLRAQGAEI |
| | protein | SEMNSPSRRVLTWLMKGVEKVIPQPVHSITEDPAQILGHGSTGDTG |
| | (GenBank | CTDEPNEALEAQDTRPGLRLLLWLEQNLERVLPQPPKSSEVWRDE |
| | NG_016351) | PAVATGAASDPAPPGRPQEMGPKLQARETPSLPTPIPLQPKEEPKE |
| | | APAPEPQPGSQAQTSSLPPTRDPARLVAWVLHRLEMALPQPVLHG |
| | | KIGEQEPDSPGICDVQTISILPGGQVEPDLVLEEVEPPWEDAHQDVS |
| | | TSPQGTEVVPAYEEENKAVEKMPRELSRIEEEKEDEEEEEEEEEEE |
| | | EEEEVTEVLLDSCVVSQVGVGQSEEDGTRPQSTSDQKLWEEVGEE |
| | | AKKEAEEKAKEEAEEVAEEEAEKEPQDWAETKEEPEAEAEAASS |
| | | GVPATKQHPEVQVEDTDADSCPLMAEENPPSTVLPPPSPAKSDTLI |
| | | VPSSASGTHRKKLPSEDDEAEELKALSPAESPVVAWSDPTTPKDTD |
| | | GQDRAASTASTNSAIINDRLQELVKLFKERTEKVKEKLIDPDVTSD |
| | | EESPKPSPAKKAPEPAPDTKPAEAEPVEEEHYCDMLCCKFKHRPW |
| | | KKYQFPQSIDPLTNLMYVLWLFFVVMAWNWNCWLIPVRWAFPY |
| | | QTPDNIHHWLLMDYLCDLIYFLDITVFQTRLQFVRGGDIITDKKDM |
| | | RNNYLKSRRFKMDLLSLLPLDFLYLKVGVNPLLRLPRCLKYMAFF |
| | | EFNSRLESILSKAYVYRVIRTTAYLLYSLHLNSCLYYWASAYQGL |
| | | GSTHWVYDGVGNSYIRCYYFAVKTLITIGGLPDPKTLFEIVFQLLN |
| | | YFTGVFAFSVMIGQMRDVVGAATAGQTYYRSCMDSTVKYMNFY |
| | | KIPKSVQNRVKTWYEYTWHSQGMLDESELMVQLPDKMRLDLAID |
| | | VNYNIVSKVALFQGCDRQMIFDMLKRLRSVVYLPNDYVCKKGEI |
| | | GREMYIIQAGQVQVLGGPDGKSVLVTLKAGSVFGEISLLAVGGGN |
| | | RRTANVVAHGFTNLFILDKKDLNEILVHYPESQKLLRKKARRMLR |
| | | SNNKPKEEKSVLILPPRAGTPKLFNAALAMTGKMGGKGAKGGKL |
| | | AHLRARLKELAALEAAAKQQELVEQAKSSQDVKGEEGSAAPDQH |
| | | THPKEAATDPPAPRTPPEPPGSPPSSPPPASLGRPEGEEEGPAEPEEH |
| | | SVRICMSPGPEPGEQILSVKMPEEREEKAE |
| 42 | Sequence of | CTGCGCGCTCGCTCGCTCACTGAGGCCGCCCGGGCAAAGCCCG |
| | 5′ITR- | GGCGTCGGGCGACCTTTGGTCGCCCGGCCTCAGTGAGCGAGCG |
| | hRHO | AGCGCGCAGAGAGGGAGTGGCCAACTCCATCACTAGGGGTTCC |
| | promoter- | TTGTAGTTAATGATTAACCCGCCATGCTACTTATCTACGTAGCC |
| | CNGB1a- | ATGCTCTAGGAAGATCGGAATTCGCCCTTAAGCCTCTCCTCCCT |
| | SV40polyA- | GACCTCAGGCTTCCTCCTAGTGTCACCTTGGCCCCTCTTAGAAG |
| | 3′ITR | CCAATTAGGCCCTCAGTTTCTGCAGCGGGGATTAATATGATTAT |
| | | GAACACCCCCAATCTCCCAGATGCTGATTCAGCCAGGAGCTTA |
| | | GGAGGGGGAGGTCACTTTATAAGGGTCTGGGGGGGTCAGAACC |
| | | CAGAGTCATCACTAGTAACGGCCGCCAGTGTGCTGGAATTCGC |
| | | CCTTCTCCACCGCCATGTTGGGCTGGGTCCAGAGGGTGCTGCCT |
| | | CAGCCCCCAGGGACCCCTCGGAAGACCAAGATGCAGGAGGAA |
| | | GAGGAAGTGGAACCAGAGCCAGAGATGGAGGCGGAGGTGGAA |
| | | CCAGAACCGAATCCTGAGGAGGCCGAGACAGAGTCCGAGTCCA |
| | | TGCCCCCCGAAGAGTCATTCAAGGAGGAGGAAGTGGCTGTGGC |
| | | AGACCCAAGCCCTCAGGAGACCAAGGAGGCTGCCCTTACTTCC |
| | | ACCATATCCCTCCGGGCCCAGGGCGCTGAGATTTCTGAAATGA |
| | | ATAGTCCCAGCCACAGGGTACTGACCTGGCTCATGAAGGGTGT |
| | | AGAGAAGGTGATCCCGCAGCCTGTTCACAGCATCACGGAGGAC |
| | | CCGGCTCAGATCCTGGGGCATGGCAGCACTGGGGACACAGGGT |
| | | GCACAGATGAACCCAATGAGGCCCTTGAGGCCCAAGACACTAG |
| | | GCCTGGGCTGCGGCTGCTTCTGTGGCTGGAGCAGAATCTGGAA |
| | | AGAGTGCTTCCTCAGCCCCCCAAATCCTCTGAGGTCTGGAGAG |
| | | ATGAGCCTGCAGTTGCTACAGCGCCTCCAGGACGCCCCCAGGA |
| | | AATGGGGCCCAAGCTGCAGGCCCGGGAGACCCCCTCCCTGCCC |
| | | ACACCCATCCCCCTGCAGCCCAAGGAGGAACCCAAGGAGGCAC |
| | | CAGCTCCAGAGCCCCAGCCCGGCTCCCAGGCCCAGACCTCCTC |
| | | CCTGCCACCAACCAGGGACCCTGCCAGGCTGGTGGCATGGGTC |
| | | CTGCACAGGCTGGAGATGGCCTTGCCGCAGCCAGTGCTACATG |
| | | GGAAAATAGGGGAACAGGAGCCTGACTCCCCTGGGATATGTGA |
| | | TGTGCAGACCATCAGCATCCTTCCTGGAGGACAAGTGGAGCCT |
| | | GACCTTGTCCTAGAGGAGGTTGAACCGCCCTGGGAGGATGCCC |
| | | ACCAGGATGTCAGTACCAGCCCACAGGGTACAGAGGTGGTTCC |
| | | AGCTTATGAAGAAGAGAACAAAGCTGTGGAGAAGATGCCCAG |
| | | AGAGCTGTCCCGGATTGAAGAGGAGAAAGAAGATGAGGAGGA |
| | | GGAAGAGGAAGAGGAGGAGGAGGAGGAAGAGGAGGAGGTGA |
| | | CTGAGGTGCTGCTGGATAGCTGTGTGGTGTCGCAGGTGGGCGT |
| | | GGGCCAGAGTGAAGAAGACGGGACCCGGCCCCAGAGCACTTC |
| | | AGATCAGAAGCTGTGGGAGGAAGTTGGGGAGGAGGCCAAGAA |
| | | GGAGGCTGAAGAGAAGGCCAAGGAGGAGGCCGAGGAGGTGGC |
| | | TGAAGAGGAGGCTGAAAAGGAGCCCCAGGACTGGGCGGAGAC |
| | | CAAGGAGGAGCCTGAGGCTGAGGCCGAGGCTGCCAGTTCAGG |
| | | AGTGCCTGCCACGAAACAGCACCCAGAAGTGCAGGTGGAAGAT |
| | | ACTGATGCTGATAGCTGCCCCCTCATGGCAGAAGAGAATCCAC |
| | | CCTCAACCGTGTTGCCGCCACCATCTCCTGCCAAATCAGACACC |
| | | CTTATAGTCCCAAGCTCAGCCTCGGGGACACACAGGAAGAAGC |
| | | TGCCCTCTGAGGATGATGAGGCTGAAGAGCTCAAGGCGTTGTC |
| | | ACCAGCAGAGTCCCCAGTGGTTGCCTGGTCTGACCCCACCACC |
| | | CCGAAGGACACTGATGGCCAGGACCGTGCGGCCTCCACGGCCA |
| | | GCACAAATAGCGCCATCATCAACGACCGGCTCCAGGAGCTGGT |
| | | GAAGCTCTTCAAGGAGCGGACAGAGAAAGTGAAGGAGAAACT |
| | | CATTGACCCTGACGTCACCTCTGATGAGGAGAGCCCCAAGCCC |
| | | TCCCCAGCCAAGAAAGCCCCAGAGCCAGCTCCAGACACAAAGC |
| | | CCGCTGAAGCCGAGCCAGTGGAAGAGGAGCACTATTGCGACAT |
| | | GCTCTGCTGCAAGTTCAAACACCGCCCCTGGAAGAAGTACCAG |
| | | TTTCCCCAGAGCATTGACCCGCTGACCAACCTGATGTATGTCCT |
| | | ATGGCTGTTCTTCGTGGTGATGGCCTGGAATTGGAACTGTTGGC |
| | | TGATTCCCGTGCGCTGGGCCTTCCCCTACCAGACCCCGGACAAC |
| | | ATCCACCACTGGCTGCTGATGGATTACCTATGCGACCTCATCTA |
| | | CTTCCTGGACATCACCGTGTTCCAGACACGCCTGCAGTTTGTCA |
| | | GAGGCGGGGACATCATTACGGACAAAAAGGACATGCGAAATA |
| | | ACTACCTGAAGTCTCGCCGCTTCAAGATGGACCTGCTCAGCCTC |
| | | CTGCCCTTGGATTTTCTCTATTTGAAAGTCGGTGTGAACCCCCT |
| | | CCTCCGCCTGCCCCGCTGTTTAAAGTACATGGCCTTCTTCGAGT |
| | | TTAACAGCCGCCTGGAATCCATCCTCAGCAAAGCCTACGTGTA |
| | | CAGGGTCATCAGGACCACAGCCTACCTTCTCTACAGCCTGCATT |
| | | TGAATTCCTGTCTTTATTACTGGGCATCGGCCTATCAGGGCCTC |
| | | GGCTCCACTCACTGGGTTTACGATGGCGTGGGAAACAGTTATA |
| | | TTCGCTGTTACTACTTTGCTGTGAAGACCCTCATCACCATCGGG |
| | | GGGCTGCCTGACCCCAAGACACTCTTTGAAATTGTCTTCCAGCT |
| | | GCTGAATTATTTCACGGGCGTCTTTGCTTTCTCTGTGATGATCG |
| | | GACAGATGAGAGATGTGGTAGGGGCCGCCACCGCGGGACAGA |
| | | CCTACTACCGCAGCTGCATGGACAGCACGGTGAAGTACATGAA |
| | | TTTCTACAAGATCCCCAAGTCCGTGCAGAACCGCGTCAAGACC |
| | | TGGTACGAGTACACCTGGCACTCGCAAGGCATGCTGGATGAGT |
| | | CAGAGCTGATGGTGCAGCTTCCAGACAAGATGCGGCTGGACCT |
| | | CGCCATCGACGTGAACTACAACATCGTTAGCAAAGTCGCACTC |
| | | TTTCAGGGCTGTGACCGGCAGATGATCTTTGACATGCTGAAGA |
| | | GGCTTCGCTCTGTTGTCTACCTGCCCAACGACTATGTGTGCAAG |
| | | AAGGGGGAGATCGGCCGTGAGATGTACATCATCCAGGCAGGGC |
| | | AAGTGCAGGTCTTGGGCGGCCCTGATGGGAAATCTGTGCTGGT |
| | | GACGCTGAAAGCTGGATCTGTGTTTGGAGAAATAAGCTTGCTG |
| | | GCTGTTGGGGGCGGGAACCGGCGCACGGCCAACGTGGTGGCGC |
| | | ACGGGTTTACCAACCTCTTCATCCTGGATAAGAAGGACCTGAA |
| | | TGAGATTTTGGTGCATTATCCTGAGTCTCAGAAGTTACTCCGGA |
| | | AGAAAGCCAGGCGCATGCTGAGAAGCAACAATAAGCCCAAGG |
| | | AGGAGAAGAGCGTGCTGATCCTTCCACCCCGGGCGGGCACCCC |
| | | AAAGCTCTTCAACGCTGCCCTCGCTATGACAGGAAAGATGGGT |
| | | GGCAAGGGGGCAAAAGGCGGCAAACTTGCTCACCTCCGGGCCC |
| | | GGCTCAAAGAACTGGCCGCGCTGGAGGCGGCTGCAAAGCAGC |
| | | AAGAGTTGGTGGAACAGGCCAAGAGCTCGCAAGACGTCAAGG |
| | | GAGAGGAAGGCTCCGCCGCCCCAGACCAGCACACGCACCCAA |
| | | AGGAGGCCGCCACCGACCCACCCGCGCCCCGGACGCCCCCCGA |
| | | GCCCCCGGGGTCTCCACCGAGCTCTCCACCGCCTGCCTCCCTTG |
| | | GGAGGCCGGAGGGAGAGGAGGAGGGGCCGGCCGAGCCCGAAG |
| | | AGCACTCGGTGAGGATCTGCATGAGCCCGGGCCCGGAGCCGGG |
| | | AGAGCAGATCCTGTCGGTGAAGATGCCGGAGGAAAGGGAGGA |
| | | GAAGGCGGAGTAAGGTGGGGTGAGGCGGATCCATGGCCGCAG |
| | | ACATGATAAGATACATTGATGAGTTTGGACAAACCACAACTAG |
| | | AATGCAGTGAAAAAAATGCTTTATTTGTGAAATTTGTGATGCTA |
| | | TTGCTTTATTTGTAACCATTATAAGCTGCAATAAACAAGTTAAC |
| | | AACAACAATTGCATTCATTTTATGTTTCAGGTTCAGGGGGAGGT |
| | | GTGGGAGGTTTTTTAAAGCAAGTAAAACCTCTACAAATGTGGT |
| | | ACTCGAGTTAAGGGCGAATTCCCGATAAGGATCTTCCTAGAGC |
| | | ATGGCTACGTAGATAAGTAGCATGGCGGGTTAATCATTAACTA |
| | | CAAGGAACCCCTAGTGATGGAGTTGGCCACTCCCTCTCTGCGC |
| | | GCTCGCTCGCTCACTGAGGCCGGGCGACCAAAGGTCGCCCGAC |
| | | GCCCGGGCTTTGCCCGGGCGGCCTCAGTGAGCGAGCGAGCGCG |
| | | CAG |
| 43 | Sequence of | CTGCGCGCTCGCTCGCTCACTGAGGCCGCCCGGGCAAAGCCCG |
| | 5′ITR- | GGCGTCGGGCGACCTTTGGTCGCCCGGCCTCAGTGAGCGAGCG |
| | hRHO | AGCGCGCAGAGAGGGAGTGGCCAACTCCATCACTAGGGGTTCC |
| | promoter- | TTGTAGTTAATGATTAACCCGCCATGCTACTTATCTACGTAGCC |
| | CNGB1a- | ATGCTCTAGGAAGATCGGAATTCGCCCTTAAGCCTCTCCTCCCT |
| | SV40polyA- | GACCTCAGGCTTCCTCCTAGTGTCACCTTGGCCCCTCTTAGAAG |
| | 3′ITR | CCAATTAGGCCCTCAGTTTCTGCAGCGGGGATTAATATGATTAT |
| | (NGS) | GAACACCCCCAATCTCCCAGATGCTGATTCAGCCAGGAGCTTA |
| | | GGAGGGGGAGGTCACTTTATAAGGGTCTGGGGGGGTCAGAACC |
| | | CAGAGTCATCACTAGTAACGGCCGCCAGTGTGCTGGAATTCGC |
| | | CCTTCTCCACCGCCATGTTGGGCTGGGTCCAGAGGGTGCTGCCT |
| | | CAGCCCCCAGGGACCCCTCGGAAGACCAAGATGCAGGAGGAA |
| | | GAGGAAGTGGAACCAGAGCCAGAGATGGAGGCGGAGGTGGAA |
| | | CCAGAACCGAATCCTGAGGAGGCCGAGACAGAGTCCGAGTCCA |
| | | TGCCCCCCGAAGAGTCATTCAAGGAGGAGGAAGTGGCTGTGGC |
| | | AGACCCAAGCCCTCAGGAGACCAAGGAGGCTGCCCTTACTTCC |
| | | ACCATATCCCTCCGGGCCCAGGGCGCTGAGATTTCTGAAATGA |
| | | ATAGTCCCAGCCACAGGGTACTGACCTGGCTCATGAAGGGTGT |
| | | AGAGAAGGTGATCCCGCAGCCTGTTCACAGCATCACGGAGGAC |
| | | CCGGCTCAGATCCTGGGGCATGGCAGCACTGGGGACACAGGGT |
| | | GCACAGATGAACCCAATGAGGCCCTTGAGGCCCAAGACACTAG |
| | | GCCTGGGCTGCGGCTGCTTCTGTGGCTGGAGCAGAATCTGGAA |
| | | AGAGTGCTTCCTCAGCCCCCCAAATCCTCTGAGGTCTGGAGAG |
| | | ATGAGCCTGCAGTTGCTACAGGTGCTGCCTCAGACCCAGCGCC |
| | | TCCAGGACGCCCCCAGGAAATGGGGCCCAAGCTGCAGGCCCGG |
| | | GAGACCCCCTCCCTGCCCACACCCATCCCCCTGCAGCCCAAGG |
| | | AGGAACCCAAGGAGGCACCAGCTCCAGAGCCCCAGCCCGGCTC |
| | | CCAGGCCCAGACCTCCTCCCTGCCACCAACCAGGGACCCTGCC |
| | | AGGCTGGTGGCATGGGTCCTGCACAGGCTGGAGATGGCCTTGC |
| | | CGCAGCCAGTGCTACATGGGAAAATAGGGGAACAGGAGCCTG |
| | | ACTCCCCTGGGATATGTGATGTGCAGACCATCAGCATCCTTCCT |
| | | GGAGGACAAGTGGAGCCTGACCTTGTCCTAGAGGAGGTTGAAC |
| | | CGCCCTGGGAGGATGCCCACCAGGATGTCAGTACCAGCCCACA |
| | | GGGTACAGAGGTGGTTCCAGCTTATGAAGAAGAGAACAAAGCT |
| | | GTGGAGAAGATGCCCAGAGAGCTGTCCCGGATTGAAGAGGAG |
| | | AAAGAAGATGAGGAGGAGGAAGAGGAAGAGGAGGAGGAGGA |
| | | GGAAGAGGAGGAGGTGACTGAGGTGCTGCTGGATAGCTGTGTG |
| | | GTGTCGCAGGTGGGCGTGGGCCAGAGTGAAGAAGACGGGACC |
| | | CGGCCCCAGAGCACTTCAGATCAGCTGTGGGAGGAAGTTGGGG |
| | | AGGAGGCCAAGAAGGAGGCTGAAGAGAAGGCCAAGGAGGAG |
| | | GCCGAGGAGGTGGCTGAAGAGGAGGCTGAAAAGGAGCCCCAG |
| | | GACTGGGCGGAGACCAAGGAGGAGCCTGAGGCTGAGGCCGAG |
| | | GCTGCCAGTTCAGGAGTGCCTGCCACGAAACAGCACCCAGAAG |
| | | TGCAGGTGGAAGATACTGATGCTGATAGCTGCCCCCTCATGGC |
| | | AGAAGAGAATCCACCCTCAACCGTGTTGCCGCCACCGTCTCCT |
| | | GCCAAATCAGACACCCTTATAGTCCCAAGCTCAGCCTCGGGGA |
| | | CACACAGGAAGAAGCTGCCCTCTGAGGATGATGAGGCTGAAGA |
| | | GCTCAAGGCGTTGTCACCAGCAGAGTCCCCAGTGGTTGCCTGG |
| | | TCTGACCCCACCACCCCGAAGGACACTGATGGCCAGGACCGTG |
| | | CGGCCTCCACGGCCAGCACAAATAGCGCCATCATCAACGACCG |
| | | GCTCCAGGAGCTGGTGAAGCTCTTCAAGGAGCGGACAGAGAAA |
| | | GTGAAGGAGAAACTCATTGACCCTGACGTCACCTCTGATGAGG |
| | | AGAGCCCCAAGCCCTCCCCAGCCAAGAAAGCCCCAGAGCCAGC |
| | | TCCAGACACAAAGCCCGCTGAAGCCGAGCCAGTGGAAGAGGA |
| | | GCACTATTGCGACATGCTCTGCTGCAAGTTCAAACACCGCCCCT |
| | | GGAAGAAGTACCAGTTTCCCCAGAGCATTGACCCGCTGACCAA |
| | | CCTGATGTATGTCCTATGGCTGTTCTTCGTGGTGATGGCCTGGA |
| | | ATTGGAACTGTTGGCTGATTCCCGTGCGCTGGGCCTTCCCCTAC |
| | | CAGACCCCGGACAACATCCACCACTGGCTGCTGATGGATTACC |
| | | TATGCGACCTCATCTACTTCCTGGACATCACCGTGTTCCAGACA |
| | | CGCCTGCAGTTTGTCAGAGGCGGGGACATCATTACGGACAAAA |
| | | AGGACATGCGAAATAATTACCTGAAGTCTCGCCGCTTCAAGAT |
| | | GGACCTGCTCAGCCTCCTGCCCTTGGATTTTCTCTATTTGAAAG |
| | | TCGGTGTGAACCCCCTCCTCCGCCTGCCCCGCTGTTTAAAGTAC |
| | | ATGGCCTTCTTCGAGTTTAACAGCCGCCTGGAATCCATCCTCAG |
| | | CAAAGCCTACGTGTACAGGGTCATCAGGACCACAGCCTACCTT |
| | | CTCTACAGCCTGCATTTGAATTCCTGTCTTTATTACTGGGCATC |
| | | GGCCTATCAGGGCCTCGGCTCCACTCACTGGGTTTACGATGGCG |
| | | TGGGAAACAGTTATATTCGCTGTTACTACTTTGCTGTGAAGACC |
| | | CTCATCACCATCGGGGGGCTGCCTGACCCCAAGACACTCTTTGA |
| | | AATTGTCTTCCAGCTGCTGAATTATTTCACGGGCGTCTTTGCTTT |
| | | CTCTGTGATGATCGGACAGATGAGAGATGTGGTAGGGGCCGCC |
| | | ACCGCGGGACAGACCTACTACCGCAGCTGCATGGACAGCACGG |
| | | TGAAGTACATGAATTTCTACAAGATCCCCAAGTCCGTGCAGAA |
| | | CCGCGTCAAGACCTGGTACGAGTACACCTGGCACTCGCAAGGC |
| | | ATGCTGGATGAGTCAGAGCTGATGGTGCAGCTTCCAGACAAGA |
| | | TGCGGCTGGACCTCGCCATCGACGTGAACTACAACATCGTTAG |
| | | CAAAGTCGCACTCTTTCAGGGCTGTGACCGGCAGATGATCTTTG |
| | | ACATGCTGAAGAGGCTTCGCTCTGTTGTCTACCTGCCCAACGAC |
| | | TATGTGTGCAAGAAGGGGGAGATCGGCCGTGAGATGTACATCA |
| | | TCCAGGCAGGGCAAGTGCAGGTCTTGGGCGGCCCTGATGGGAA |
| | | ATCTGTGCTGGTGACGCTGAAAGCTGGATCTGTGTTTGGAGAA |
| | | ATAAGCTTGCTGGCTGTTGGGGGCGGGAACCGGCGCACGGCCA |
| | | ACGTGGTGGCGCACGGGTTTACCAACCTCTTCATCCTGGATAAG |
| | | AAGGACCTGAATGAGATTTTGGTGCATTATCCTGAGTCTCAGA |
| | | AGTTACTCCGGAAGAAAGCCAGGCGCATGCTGAGAAGCAACA |
| | | ATAAGCCCCAGGAGGAGAAGAGCGTGCTGATCCTTCCACCCCG |
| | | GGCGGGCACCCCAAAGCTCTTCAACGCTGCCCTCGCTATGACA |
| | | GGAAAGATGGGTGGCAAGGGGGCAAAAGGCGGCAAACTTGCT |
| | | CACCTCCGGGCCCGGCTCAAAGAACTGGCCGCGCTGGAGGCGG |
| | | CTGCAAAGCAGCAAGAGTTGGTGGAACAGGCCAAGAGCTCGC |
| | | AAGACGTCAAGGGAGAGGAAGGCTCCGCCGCCCCAGACCAGC |
| | | ACACGCACCCAAAGGAGGCCGCCACCGACCCACCCGCGCCCCG |
| | | GACGCCCCCCGAGCCCCCGGGGTCTCCACCGAGCTCTCCACCG |
| | | CCTGCCTCCCTTGGGAGGCCGGAGGGAGAGGAGGAGGGGCCG |
| | | GCCGAGCCCGAAGAGCACTCGGTGAGGATCTGCATGAGCCCGG |
| | | GCCCGGAGCCGGGAGAGCAGATCCTGTCGGTGAAGATGCCGGA |
| | | GGAAAGGGAGGAGAAGGCGGAGTAAGGTGGGGTGAGGCGGAT |
| | | CCATGGCCGCAGACATGATAAGATACATTGATGAGTTTGGACA |
| | | AACCACAACTAGAATGCAGTGAAAAAAATGCTTTATTTGTGAA |
| | | ATTTGTGATGCTATTGCTTTATTTGTAACCATTATAAGCTGCAA |
| | | TAAACAAGTTAACAACAACAATTGCATTCATTTTATGTTTCAGG |
| | | TTCAGGGGGAGGTGTGGGAGGTTTTTTAAAGCAAGTAAAACCT |
| | | CTACAAATGTGGTCTCGAGTTAAGGGCGAATTCCCGATAAGGA |
| | | TCTTCCTAGAGCATGGCTACGTAGATAAGTAGCATGGCGGGTT |
| | | AATCATTAACTACAAGGAACCCCTAGTGATGGAGTTGGCCACT |
| | | CCCTCTCTGCGCGCTCGCTCGCTCACTGAGGCCGGGCGACCAA |
| | | AGGTCGCCCGACGCCCGGGCTTTGCCCGGGCGGCCTCAGTGAG |
| | | CGAGCGAGCGCGCAG |
| 44 | Sequence of | GGCCACTCCCTCTCTGCGCGCTCGCTCGCTCACTGAGGCCGCCC |
| | 5′ITR- | GGGCAAAGCCCGGGCGTCGGGCGACCTTTGGTCGCCCGGCCTC |
| | hRHO | AGTGAGCGAGCGAGCGCGCAGAGAGGGAGTGGCCAACTCCAT |
| | promoter- | CACTAGGGGTTCCTCAGATCGTAGCCATGCTCTAGGAAGATCG |
| | CNGB1a- | GAATTCGCCCTTAAGCCTCTCCTCCCTGACCTCAGGCTTCCTCC |
| | SV40polyA- | TAGTGTCACCTTGGCCCCTCTTAGAAGCCAATTAGGCCCTCAGT |
| | 3′ITR | TTCTGCAGCGGGGATTAATATGATTATGAACACCCCCAATCTCC |
| | (GenBank) | CAGATGCTGATTCAGCCAGGAGCTTAGGAGGGGGAGGTCACTT |
| | | TATAAGGGTCTGGGGGGGTCAGAACCCAGAGTCATCACTAGTA |
| | | ACGGCCGCCAGTGTGCTGGAATTCGCCCTTCTCCACCGCCATGT |
| | | TGGGCTGGGTCCAGAGGGTGCTGCCTCAGCCCCCAGGGACCCC |
| | | TCGGAAGACCAAGATGCAGGAGGAAGAGGAAGTGGAACCAGA |
| | | GCCAGAGATGGAGGCGGAGGTGGAACCAGAACCGAATCCTGA |
| | | GGAGGCCGAGACAGAGTCCGAGTCCATGCCCCCCGAAGAGTCA |
| | | TTCAAGGAGGAGGAAGTGGCTGTGGCAGACCCAAGCCCTCAGG |
| | | AGACCAAGGAGGCTGCCCTTACTTCCACCATATCCCTCCGGGCC |
| | | CAGGGCGCTGAGATTTCTGAAATGAATAGTCCCAGCCGCAGGG |
| | | TACTGACCTGGCTCATGAAGGGTGTAGAGAAGGTGATCCCGCA |
| | | GCCTGTTCACAGCATCACGGAGGACCCGGCTCAGATCCTGGGG |
| | | CATGGCAGCACTGGGGACACAGGGTGCACAGATGAACCCAATG |
| | | AGGCCCTTGAGGCCCAAGACACTAGGCCTGGGCTGCGGCTGCT |
| | | TCTGTGGCTGGAGCAGAATCTGGAAAGAGTGCTTCCTCAGCCC |
| | | CCCAAATCCTCTGAGGTCTGGAGAGATGAGCCTGCAGTTGCTA |
| | | CAGGTGCTGCCTCAGACCCAGCGCCTCCAGGACGCCCCCAGGA |
| | | AATGGGGCCCAAGCTGCAGGCCCGGGAGACCCCCTCCCTGCCC |
| | | ACACCCATCCCCCTGCAGCCCAAGGAGGAACCCAAGGAGGCAC |
| | | CAGCTCCAGAGCCCCAGCCCGGCTCCCAGGCCCAGACCTCCTC |
| | | CCTGCCACCAACCAGGGACCCTGCCAGGCTGGTGGCATGGGTC |
| | | CTGCACAGGCTGGAGATGGCCTTGCCGCAGCCAGTGCTACATG |
| | | GGAAAATAGGGGAACAGGAGCCTGACTCCCCTGGGATATGTGA |
| | | TGTGCAGACCATCAGCATCCTTCCTGGAGGACAAGTGGAGCCT |
| | | GACCTTGTCCTAGAGGAGGTTGAACCGCCCTGGGAGGATGCCC |
| | | ACCAGGATGTCAGTACCAGCCCACAGGGTACAGAGGTGGTTCC |
| | | AGCTTATGAAGAAGAGAACAAAGCTGTGGAGAAGATGCCCAG |
| | | AGAGCTGTCCCGGATTGAAGAGGAGAAAGAAGATGAGGAGGA |
| | | GGAAGAGGAAGAGGAGGAGGAGGAGGAAGAGGAGGAGGTGA |
| | | CTGAGGTGCTGCTGGATAGCTGTGTGGTGTCGCAGGTGGGCGT |
| | | GGGCCAGAGTGAAGAAGACGGGACCCGGCCCCAGAGCACTTC |
| | | AGATCAGAAGCTGTGGGAGGAAGTTGGGGAGGAGGCCAAGAA |
| | | GGAGGCTGAAGAGAAGGCCAAGGAGGAGGCCGAGGAGGTGGC |
| | | TGAAGAGGAGGCTGAAAAGGAGCCCCAGGACTGGGCGGAGAC |
| | | CAAGGAGGAGCCTGAGGCTGAGGCCGAGGCTGCCAGTTCAGG |
| | | AGTGCCTGCCACGAAACAGCACCCAGAAGTGCAGGTGGAAGAT |
| | | ACTGATGCTGATAGCTGCCCCCTCATGGCAGAAGAGAATCCAC |
| | | CCTCAACCGTGTTGCCGCCACCGTCTCCTGCCAAATCAGACACC |
| | | CTTATAGTCCCAAGCTCAGCCTCGGGGACACACAGGAAGAAGC |
| | | TGCCCTCTGAGGATGATGAGGCTGAAGAGCTCAAGGCGTTGTC |
| | | ACCAGCAGAGTCCCCAGTGGTTGCCTGGTCTGACCCCACCACC |
| | | CCGAAGGACACTGATGGCCAGGACCGTGCGGCCTCCACGGCCA |
| | | GCACAAATAGCGCCATCATCAACGACCGGCTCCAGGAGCTGGT |
| | | GAAGCTCTTCAAGGAGCGGACAGAGAAAGTGAAGGAGAAACT |
| | | CATTGACCCTGACGTCACCTCTGATGAGGAGAGCCCCAAGCCC |
| | | TCCCCAGCCAAGAAAGCCCCAGAGCCAGCTCCAGACACAAAGC |
| | | CCGCTGAAGCCGAGCCAGTGGAAGAGGAGCACTATTGCGACAT |
| | | GCTCTGCTGCAAGTTCAAACACCGCCCCTGGAAGAAGTACCAG |
| | | TTTCCCCAGAGCATTGACCCGCTGACCAACCTGATGTATGTCCT |
| | | ATGGCTGTTCTTCGTGGTGATGGCCTGGAATTGGAACTGTTGGC |
| | | TGATTCCCGTGCGCTGGGCCTTCCCCTACCAGACCCCGGACAAC |
| | | ATCCACCACTGGCTGCTGATGGATTACCTATGCGACCTCATCTA |
| | | CTTCCTGGACATCACCGTGTTCCAGACACGCCTGCAGTTTGTCA |
| | | GAGGCGGGGACATCATTACGGACAAAAAGGACATGCGAAATA |
| | | ACTACCTGAAGTCTCGCCGCTTCAAGATGGACCTGCTCAGCCTC |
| | | CTGCCCTTGGATTTTCTCTATTTGAAAGTCGGTGTGAACCCCCT |
| | | CCTCCGCCTGCCCCGCTGTTTAAAGTACATGGCCTTCTTCGAGT |
| | | TTAACAGCCGCCTGGAATCCATCCTCAGCAAAGCCTACGTGTA |
| | | CAGGGTCATCAGGACCACAGCCTACCTTCTCTACAGCCTGCATT |
| | | TGAATTCCTGTCTTTATTACTGGGCATCGGCCTATCAGGGCCTC |
| | | GGCTCCACTCACTGGGTTTACGATGGCGTGGGAAACAGTTATA |
| | | TTCGCTGTTACTACTTTGCTGTGAAGACCCTCATCACCATCGGG |
| | | GGGCTGCCTGACCCCAAGACACTCTTTGAAATTGTCTTCCAGCT |
| | | GCTGAATTATTTCACGGGCGTCTTTGCTTTCTCTGTGATGATCG |
| | | GACAGATGAGAGATGTGGTAGGGGCCGCCACCGCGGGACAGA |
| | | CCTACTACCGCAGCTGCATGGACAGCACGGTGAAGTACATGAA |
| | | TTTCTACAAGATCCCCAAGTCCGTGCAGAACCGCGTCAAGACC |
| | | TGGTACGAGTACACCTGGCACTCGCAAGGCATGCTGGATGAGT |
| | | CAGAGCTGATGGTGCAGCTTCCAGACAAGATGCGGCTGGACCT |
| | | CGCCATCGACGTGAACTACAACATCGTTAGCAAAGTCGCACTC |
| | | TTTCAGGGCTGTGACCGGCAGATGATCTTTGACATGCTGAAGA |
| | | GGCTTCGCTCTGTTGTCTACCTGCCCAACGACTATGTGTGCAAG |
| | | AAGGGGGAGATCGGCCGTGAGATGTACATCATCCAGGCAGGGC |
| | | AAGTGCAGGTCTTGGGCGGCCCTGATGGGAAATCTGTGCTGGT |
| | | GACGCTGAAAGCTGGATCTGTGTTTGGAGAAATAAGCTTGCTG |
| | | GCTGTTGGGGGCGGGAACCGGCGCACGGCCAACGTGGTGGCGC |
| | | ACGGGTTTACCAACCTCTTCATCCTGGATAAGAAGGACCTGAA |
| | | TGAGATTTTGGTGCATTATCCTGAGTCTCAGAAGTTACTCCGGA |
| | | AGAAAGCCAGGCGCATGCTGAGAAGCAACAATAAGCCCAAGG |
| | | AGGAGAAGAGCGTGCTGATCCTTCCACCCCGGGCGGGCACCCC |
| | | AAAGCTCTTCAACGCTGCCCTCGCTATGACAGGAAAGATGGGT |
| | | GGCAAGGGGGCAAAAGGCGGCAAACTTGCTCACCTCCGGGCCC |
| | | GGCTCAAAGAACTGGCCGCGCTGGAGGCGGCTGCAAAGCAGC |
| | | AAGAGTTGGTGGAACAGGCCAAGAGCTCGCAAGACGTCAAGG |
| | | GAGAGGAAGGCTCCGCCGCCCCAGACCAGCACACGCACCCAA |
| | | AGGAGGCCGCCACCGACCCACCCGCGCCCCGGACGCCCCCCGA |
| | | GCCCCCGGGGTCTCCACCGAGCTCTCCACCGCCTGCCTCCCTTG |
| | | GGAGGCCGGAGGGAGAGGAGGAGGGGCCGGCCGAGCCCGAAG |
| | | AGCACTCGGTGAGGATCTGCATGAGCCCGGGCCCGGAGCCGGG |
| | | AGAGCAGATCCTGTCGGTGAAGATGCCGGAGGAAAGGGAGGA |
| | | GAAGGCGGAGTAAGGTGGGGTGAGGCGGATCCATGGCCGCAG |
| | | ACATGATAAGATACATTGATGAGTTTGGACAAACCACAACTAG |
| | | AATGCAGTGAAAAAAATGCTTTATTTGTGAAATTTGTGATGCTA |
| | | TTGCTTTATTTGTAACCATTATAAGCTGCAATAAACAAGTTAAC |
| | | AACAACAATTGCATTCATTTTATGTTTCAGGTTCAGGGGGAGGT |
| | | GTGGGAGGTTTTTTAAAGCAAGTAAAACCTCTACAAATGTGGT |
| | | CTCGAGTTAAGGGCGAATTCCCGATAAGGATCTTCCTAGAGCA |
| | | TGGCTACGATCTGAGGAACCCCTAGTGATGGAGTTGGCCACTC |
| | | CCTCTCTGCGCGCTCGCTCGCTCACTGAGGCCGGGCGACCAAA |
| | | GGTCGCCCGACGCCCGGGCTTTGCCCGGGCGGCCTCAGTGAGC |
| | | GAGCGAGCGCGCAGAGAGGGAGTGGCCAA |
| 45 | Sequence of | MSIQVEHPAGGYKKLFETVEELSSPLTAHVTGRIPLWLTGSLLRCG |
| | human | PGLFEVGSEPFYHLFDGQALLHKFDFKEGHVTYHRRFIRTDAYVR |
| | RPE65 | AMTEKRIVITEFGTCAFPDPCKNIFSRFFSYFRGVEVTDNALVNVY |
| | protein | PVGEDYYACTETNFITKINPETLETIKQVDLCNYVSVNGATAHPHI |
| | | ENDGTVYNIGNCFGKNFSIAYNIVKIPPLQADKEDPISKSEIVVQFP |
| | | CSDRFKPSYVHSFGLTPNYIVFVETPVKINLFKFLSSWSLWGANYM |
| | | DCFESNETMGVWLHIADKKRKKYLNNKYRTSPFNLFHHINTYED |
| | | NGFLIVDLCCWKGFEFVYNYLYLANLRENWEEVKKNARKAPQPE |
| | | VRRYVLPLNIDKADTGKNLVTLPNTTATAILCSDETIWLEPEVLFS |
| | | GPRQAFEFPQINYQKYCGKPYTYAYGLGLNHFVPDRLCKLNVKT |
| | | KETWVWQEPDSYPSEPIFVSHPDALEEDDGVVLSVVVSPGAGQKP |
| | | AYLLILNAKDLSEVARAEVEINIPVTFHGLFKKS |

### EXAMPLES

### Example 1: Nucleic Acid Vector

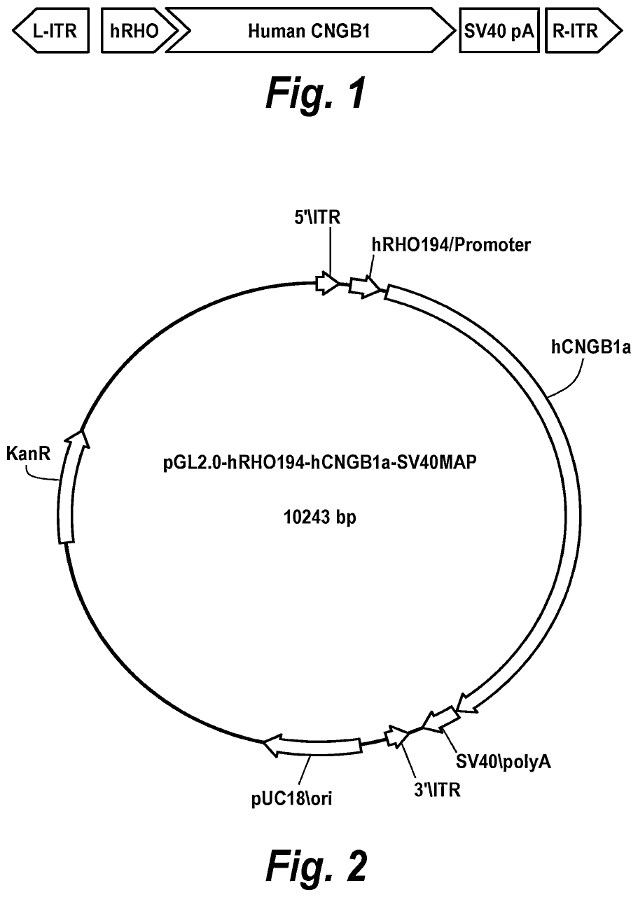

In this exemplary embodiment, the rAAV.hCNGB1 vector is a hybrid AAV-based vector carrying the cDNA of the human CNGB1 gene encoding the B subunit of the rod photoreceptor cyclic nucleotide-gated (CNG) channel. The hCNGB1 cDNA expression is under the control of the rod-specific Rhodopsin promoter (hRHO) and is enhanced using a SV40 pA sequence. The expression cassette is flanked by the AAV serotype 2 inverted terminal repeats (ITRs) and the recombinant genome is packaged in the AAV serotype 8 capsid. The expression cassette comprises the following elements:
Promoter of the human rhodopsin gene: 0.194 Kb.
cDNA of the human CNGB1a subunit of the rod photoreceptor cGMP phosphodiesterase: 3.74 Kb
Polyadenylation signal of the Simian-Virus 40 (SV40): 0.23 Kb
AAV serotype 2 inverted terminal repeats (ITRs): 0.13 KbThe structure of the rAAV.hRHO194.hCNGB1 vector genome is depicted in FIG. 1.

### Example 2: pGL2.0-hRHO194-hCNGB1a-SV40 Cis Vector Plasmid

In one exemplary embodiment, the pGL2.0-hRHO194-hCNGB1a-SV40 cis vector plasmid with the nucleotide sequence depicted in SEQ ID No. 7 is used which contains an expression cassette comprising a 194 bp rod photoreceptor-specific human rhodopsin (hRHO) promoter and the full-length (3738 bp) human CNGB1 cDNA. The expression cassette also contains a 227 bp Simian-Virus 40 polyadenylation signal (SV40 pA). The 5591 bp vector backbone containing a kanamycin resistance (KanR) positioned 1943 bp from the L-ITR and 2853 bp from the R-ITR and 2024 bp from a pUC18 ori. The rAAV.hCNGB1 vector is produced using transient co-transfection of the cis vector plasmid and trans helper plasmid(s) encoding rep and cap sequences and adenoviral genes in the human embryonic kidney 293 T cells (HEK293T). The rAAV.hRHO194.hCNGB1 is harvested from the culture medium and/or the cell lysate using standard purification methods, e.g. cesium chloride gradient ultracentrifugation, ion exchange chromatography and/or tangential flow filtration. The resulting rAAV.hRHO194.hCNGB1 vector suspension is then sterile-filtered, filled and stored as drug product.

### Example 3: Activity and Specificity of the hRHO194 Promoter

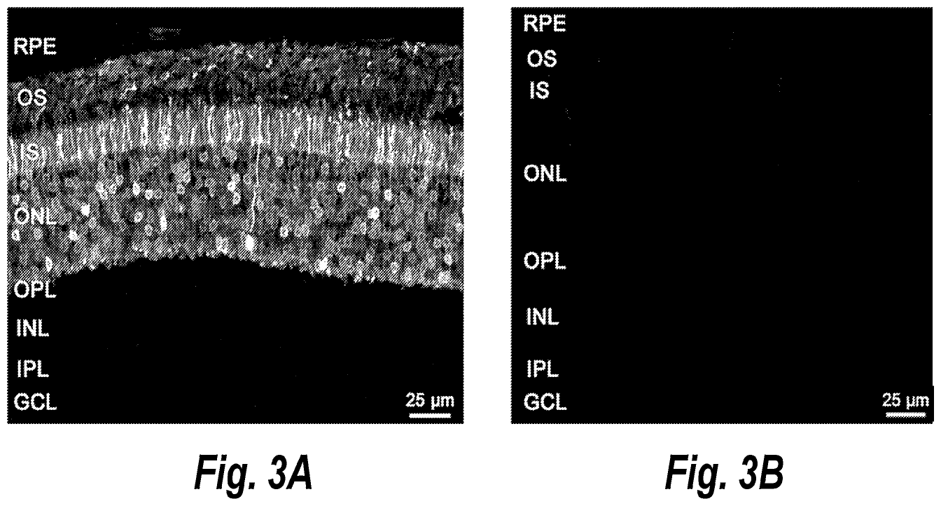

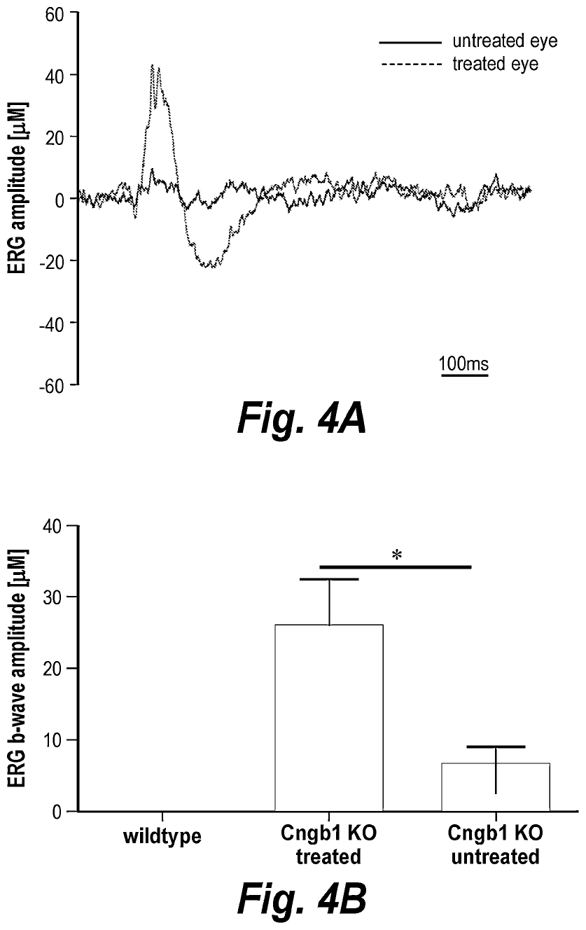

To verify the activity and specificity of the novel hRHO194 promoter the inventors constructed a version of the AAV cis vector which contains the eGFP cDNA instead of the hCNGB1 cDNA. The resulting pGL2.0-hRHO194-eGFP-SV40 cis vector plasmid map is shown in FIG. 2. Delivery of rAAV.hRHO194.eGFP vector into the subretinal space of 4-week-old wild type mice resulted in strong expression of eGFP protein 4 weeks after injection in rod photoreceptors only (FIG. 3A) thereby confirming the retinal cell type specificity of this promoter. For representative results see FIGS. 3A-3B. The rAAV.hRHO194.eGFP vector treatment resulted in strong eGFP protein expression in the treated eye reflected by native eGFP fluorescence in rod photoreceptors only.

### Example 4: Biological Activity and Transgene Expression Conferred by the rAAV.hRHO194.hCNGB1

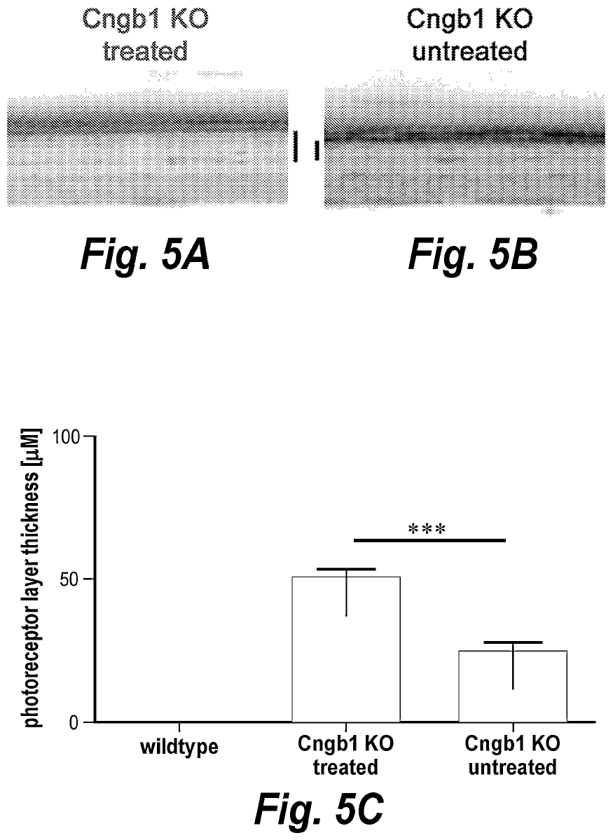

To verify biological activity and transgene expression the inventors delivered the AAV.hRHO194.hCNGB1 vector into the subretinal space of 4-week-old CNGB1 (−/−) mice. The delivery procedure was similar to the one described in Koch et al., Gene therapy restores vision and delays degeneration in the CNGB1 (−/−) mouse model of retinitis pigmentosa. Hum Mol Genet. 2012; 21 (20): 4486-96. PubMed PMID: 22802073. The mice received a subretinal injection in the treated eye (TE), whereas the other, untreated eye (UE) served as control. The vector efficacy was evaluated at 4 months following the injection by means of electroretinography (ERG), an objective functional in vivo assay (FIGS. 4A and 4B). CNGB1 (−/−) mice lack normal rod photoreceptor function. Secondary to rods, non-affected cone photoreceptors also degenerate resulting in loss of cone function at later stages of the disease. Therefore, ERG protocols specifically testing for rod and cone function are suitable as an indirect measure for CNGB1 function and for the assessment of biological activity (BAA) of the rAAV hRHO194.hCNGB1 vector.

### Example 5: In Vivo Optical Coherence Tomography (OTC) for the Determination of BAA

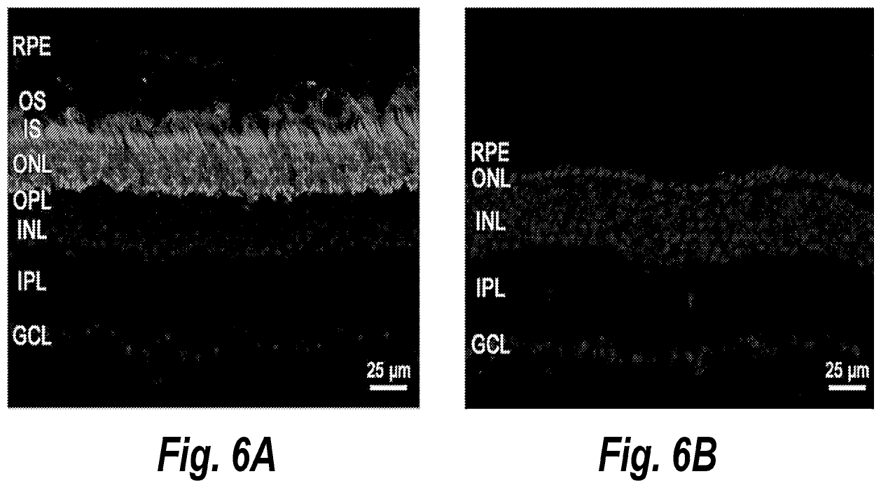

In another set of experiments BAA was determined by in vivo optical coherence tomography (OCT) imaging followed by quantification of the photoreceptor layer thickness. For this, the mice received a subretinal injection in the treated eye (TE), whereas the other, untreated eye (UE) served as control. Photoreceptor layer thickness measurement was performed at 4 months following the injection by means of OCT (FIGS. 5A-5C). Rod photoreceptors of CNGB1 (−/−) mice degenerate over time resulting in thinning of the photoreceptor cell layer (FIGS. 5B and 5C). Therefore, biological activity (BAA) of the rAAV.hRHO194.hCNGB1 vector can be indirectly measured by determining the photoreceptor layer thickness in treated CNGB1 (−/−) mice using OCT. The rAAV.hRHO194.hCNGB1 vector treatment resulted in a clear therapeutic effect in the treated eye reflected by preservation of the photoreceptor layer thickness. In particular, more than 45% increase in photoreceptor layer thickness was observed (FIG. 5C).

### Example 6: In Vivo CNGB1 Gene Augmentation in Cngb1^{-/-} Mice

Cngb1^{-/-} mice were treated with 1×10¹⁰ viral genomes (1 e 10 vgs) of AAV8-hRHO 194-hCNGB1-SV40 or AAV5-hRHO194-hCNGB1-SV40 subretinally at 4 weeks of age. Structural outcome measures included SD-OCT at 1 and 3 months post injection, histology, and immunohistochemistry.

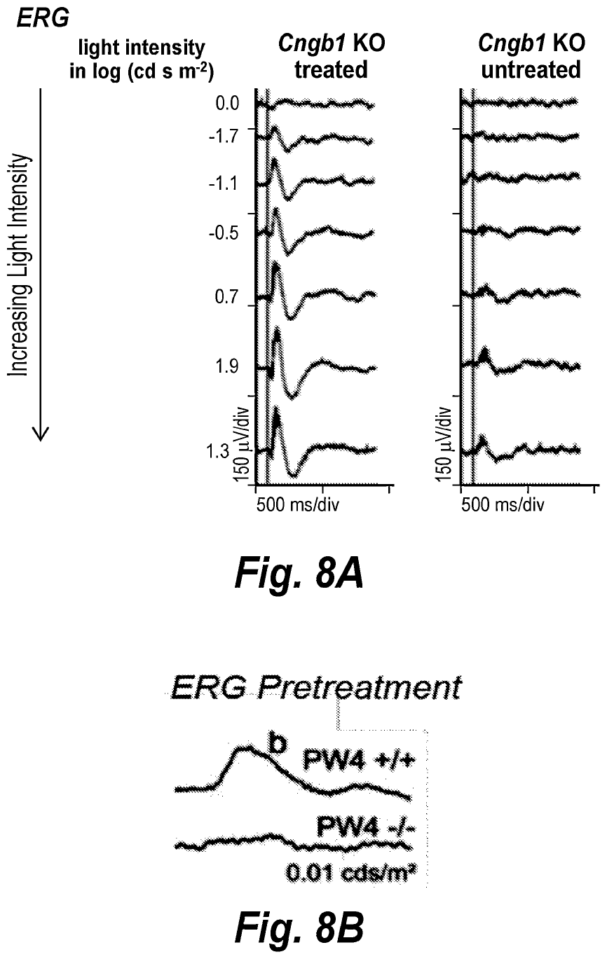

General vector design is shown in FIG. 7. 1 e 10 total viral genomes in 1 ul was injected subretinally in 4 week old Cngb1^{-/-} mice (postnatal week 4; PW4).

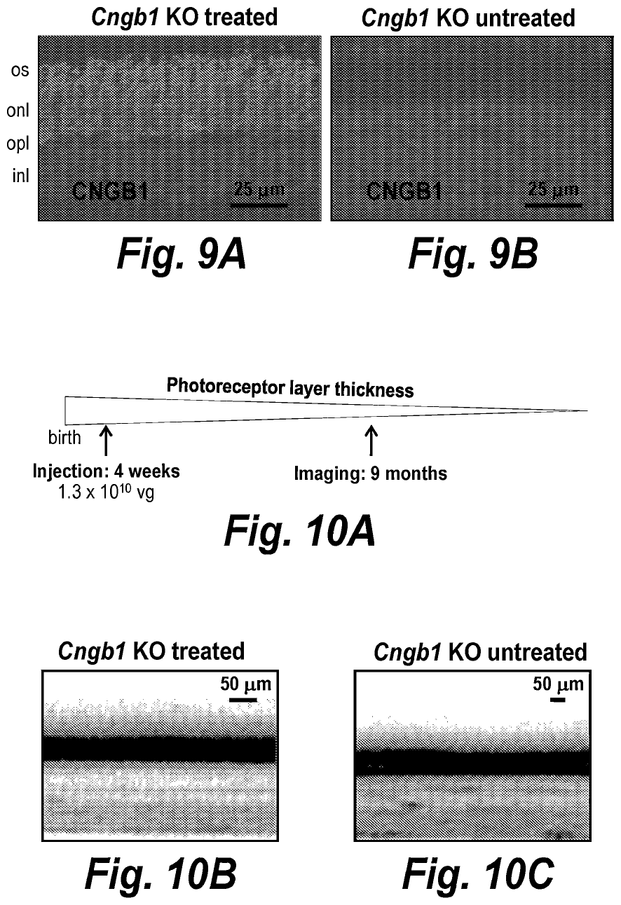

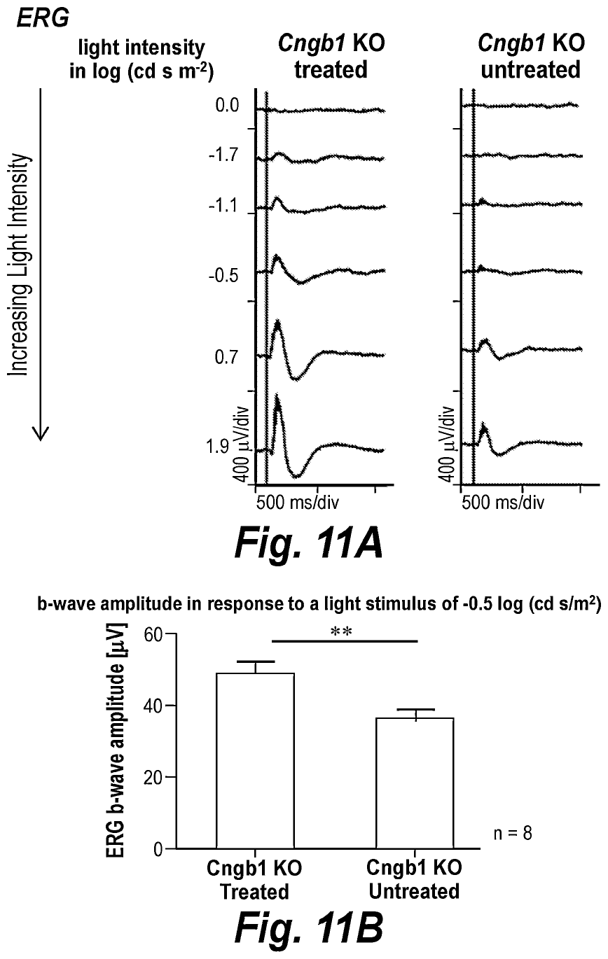

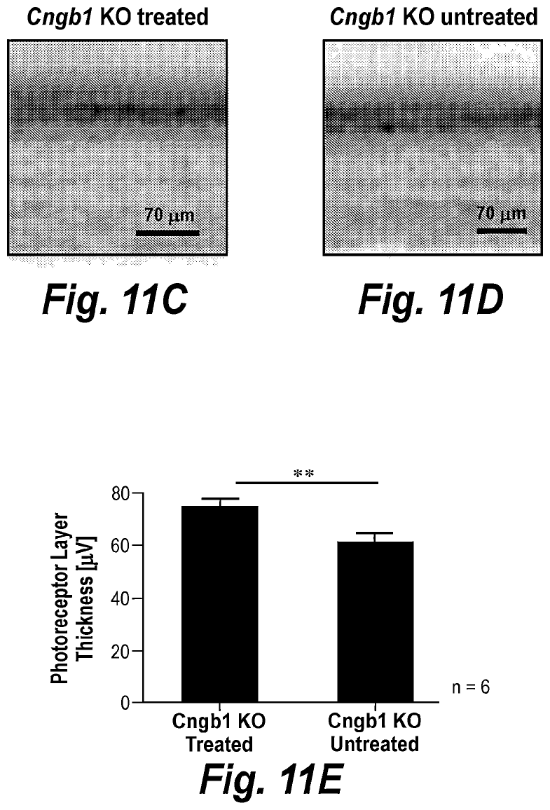

AAV8-hRHO194-hCNGB1-SV40 gene augmentation was found to result in restoration of rod function post subretinal injection in Cngb1^{-/-} mice. Efficacy was found to persist out to 8 months post-treatment. Dark adapted ERG B wave amplitudes were found to be significantly improved in the treated mice (FIGS. 8A-8B). Scotopic electroretinography (ERG) at rod-specific stimulus Cngb1^{-/-} at 9 months (8 months post treatment in treated mice) is shown in FIG. 8A. ERG of wild-type and Cngb1^{-/-} mice before treatment showed that ERG B wave was absent in Cngb1^{-/-} mice at time of injection (FIG. 8B). CNGB1 channel expression in rod outer segments was found to be restored (FIGS. 9A-9B). A rabbit polyclonal anti-CNGB1 antibody that recognizes aa 1078-1168 of human CNGB1a (Sigma-Aldrich) was used for transgene expression assays. Immunohistochemistry was performed at 9 months in Cngb1^{-/-} mice treated with AAV8-hRHO194-hCNGB1-SV40 (8 months post treatment; FIG. 9A) and in untreated Cngb1^{-/-} mice (FIG. 9B), and showed restoration of CNGB1 channel expression in treated Cngb1^{-/-} mice. OCT analysis revealed a significant delay in retinal degeneration (FIGS. 10A-10C). General injection schedule is shown in FIG. 10A. In vivo optical coherence tomography (OCT) images were collected at 9 months in Cngb1^{-/-} mice treated with AAV8-hRHO194-hCNGB1-SV40 (8 months post treatment; FIG. 10B) and in untreated Cngb1^{-/-} mice (FIG. 10C). As shown in FIG. 10, it was found that at 9 months, treated Cngb 1^{-/-} mice had a thicker photoreceptor layer compared to untreated Cngb1^{-/-} mice.

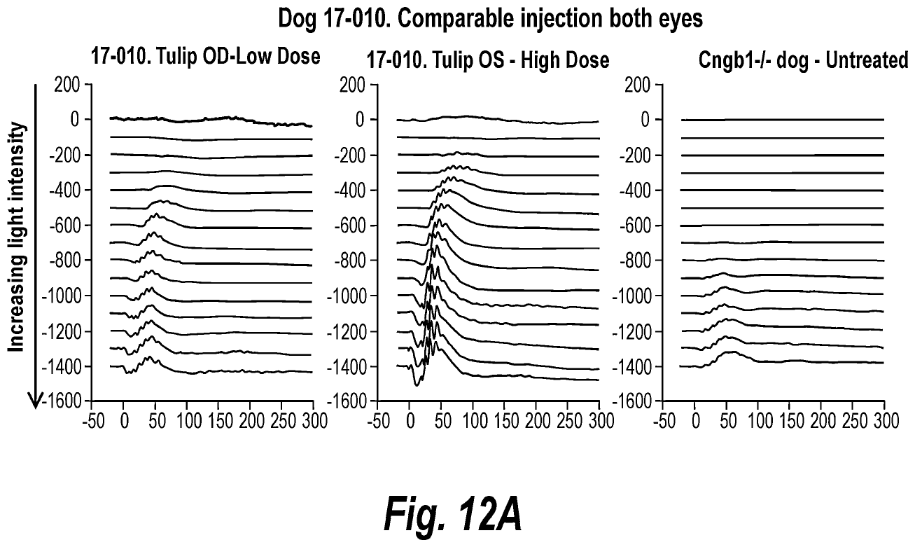

AAV5-hRHO194-hCNGB1-SV40 gene augmentation was found to result in restoration of rod function by two months post subretinal injection in Cngb1^{-/-} mice. Dark adapted ERG B wave amplitudes were found to be significantly improved in the treated mice (FIGS. 11A-11E). Scotopic ERG was measured in treated and untreated Cngb1^{-/-} mice at 3 months of age (2 months post subretinal treatment in treated mice) and results are shown in FIG. 11A. B-wave amplitude in response to a light stimulus of −0.5 log (cd s/m²) measured in treated and untreated Cngb1^{-/-} mice (n=8) at 3 months of age (2 months post subretinal treatment in treated mice) is shown in FIG. 11B. OCT analysis revealed a significant delay in retinal degeneration. In vivo optical coherence tomography (OCT) images were collected at 3 months in Cngb1^{-/-} mice treated with AAV5-hRHO194-hCNGB1-SV40 (2 months post treatment; FIG. 11C) and in untreated Cngb1^{-/-} mice (FIG. 11D). Measurement of the photoreceptor layer thickness showed a significant delay in retinal degeneration in treated Cngb1^{-/-} mice at 3 months (2 months post treatment) compared to wild-type Cngb1^{-/-} mice (n=6; FIG. 11E).

### Example 7: Mutant Dog Study Design

Cngb 1^{-/-} dogs have a mutation in exon 26 that leads to a truncated and non-functional protein, resulting in loss of rod function and retinal degeneration. Three Cngb1^{-/-} dogs were treated with AAV5-hRHO₁₉₄-hCNGB1a subretinally in both eyes at 3 months of age. For each animal, eye 1 was treated at a dose of 5 e 11 vgs (aiming for 2×100 ul blebs; “low dose”) and eye 2 was treated at a dose of 1 e 12 vgs (aiming for 2×100 ul blebs; “high dose”). Structural outcome measures included SC-OCT at 1 and 3 months post injection, histology and immunohistochemistry. Functional outcome measures included vision testing and ERG at 1 and 3 months post injection.

### Example 8: Results of Mutant Dog Studies

AAV5-hRHO194-hCNGB1a-SV40 gene augmentation was found to result in restoration of rod function by one month post subretinal injection in Cngb 1^{-/-} dogs.

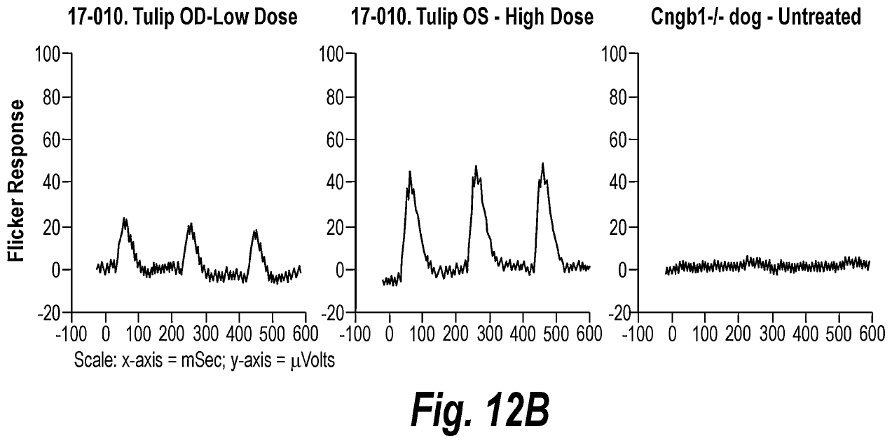

Dark adapted ERG waveforms were found to be significantly improved post treatment at both doses evaluated. Comparable injections were performed in both eyes. Obvious ERG rescue was observed in both eyes of treated dogs compared to untreated dogs, using both rod-specific stimulus (FIG. 12A) and flicker response (FIG. 12B). Larger ERG amplitudes were observed in eyes treated with the higher dose.

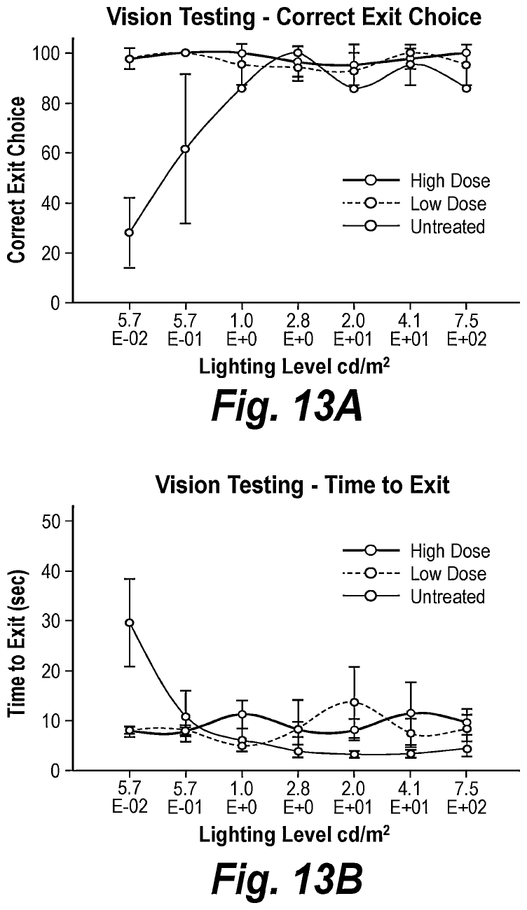

Vision testing showed that treated dogs had rod-mediated vision and improved performance in a four-choice vision testing device. FIGS. 13A-13B shows the results of vision testing of treated dogs at 1 month post injection and untreated dogs. A four-choice vision testing device was used. Untreated Cngb1^{-/-} dogs were found to have normal cone vision at this age, but lack rod-mediated vision. Untreated Cngb1^{-/-} dogs are blind at lower light levels (e.g., 5.7 e −2 cd/m²) and make fewer correct exit choices and take longer to exit from the testing device. Both treatment groups (high and low dose) were found to have restored rod vision as indicated by the significantly improved performance in correct exit choice (FIG. 13A) and time to exit (FIG. 13B), at the lowest lighting level.

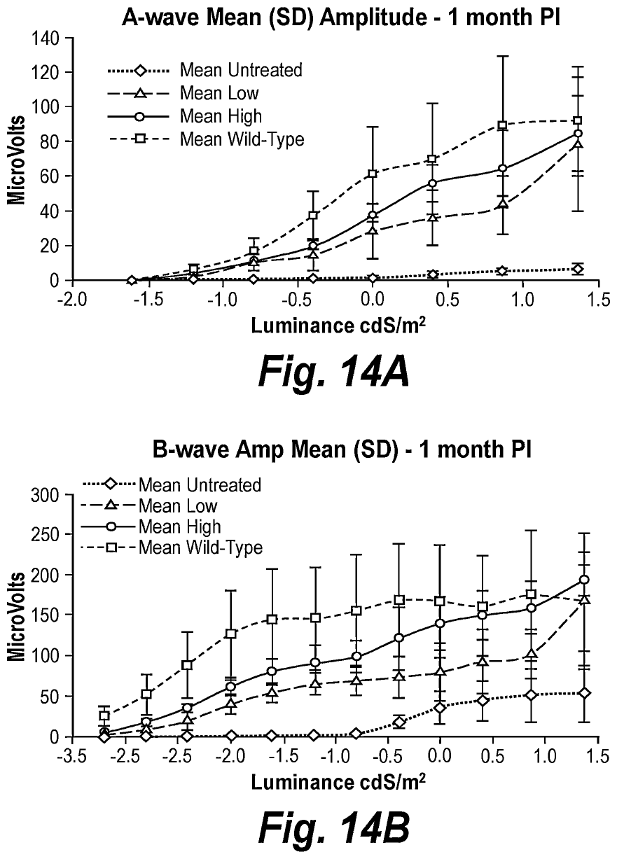

The mean ERG A- and B-wave amplitudes in the high dose group were found to be higher compared to the low dose group. A- and B-wave amplitudes in treated eyes were found to be about 80% of wild-type levels. FIGS. 14A-14B shows ERG amplitude measurements one month post injection in each treatment group and the untreated group. A highly significant increase in A-wave amplitude for both treatment groups was observed compared to untreated controls. Improvement in response threshold in treated eyes was found to be greater than 1.5 log units (FIG. 14A). A highly significant increase in B-wave amplitude for all stages in the high dose group and all but the second and third strongest stimuli for the low dose group was found, compared to untreated controls. Improvement in response threshold in treated eyes was found to be greater than 2 log units (FIG. 14B).

1. A polynucleotide comprising in this order:
a) a promoter comprising a human rod photoreceptor-specific promoter element (hRPSPE) comprising, consisting essentially of or consisting of the nucleic acid sequence according to SEQ ID NO: 1 or variants thereof and a core promoter (CP); and
b) at least one transgene (TG) operably linked to the promoter of a);
wherein the variant of SEQ ID NO: 1 comprises one or more nucleic acid substitutions outside nucleotide positions 6 to 13, 32 to 40, 70 to 83, and 87 to 94 of SEQ ID NO: 1.

2. The polynucleotide according to claim 1, wherein:
the 5′ end of the hRPSPE is at a nucleic acid position from 1 to 160 and the 3′ end at a nucleic acid position from 290 to 310 of SEQ ID NO: 2 or variants thereof;
the CP comprises a TATA-box and/or an initiator (Inr);
the 5′ end of the promoter is at a nucleic acid position from 1 to 160 and the 3′ end at a nucleic acid position from 340 to 350 of SEQ ID NO: 2 or variants thereof; and/or
the at least one transgene comprises a nucleic acid encoding a protein or RNA that maintains or improves the physiological function of rods.

3-5. (canceled)

6. The polynucleotide according to claims-1 to 5, wherein the at least one transgene:
(i) comprises a nucleic acid encoding the human rod cyclic nucleotide-gated channel beta subunit (hCNGB1), ABCA4, AIPL1, BEST1, CACNA1F, CLN3, CLRN1, CNGA1, CEP290, CRB1, CRB2, CRX, GPR98, GUCA1A, GUCA1B, MYO7A, NRL, PDE6A, PDE6B, PRPH2, PROM1, RHO, ROM1, RP1, RP2, RPE65, RPGR, SAG, USH1C, USH1G, USH2A or functional fragments or variants thereof, optionally wherein the hCNGB1 comprises an amino acid sequence according to SEQ ID NOs: 3, 40, or 41, or variants thereof;
(ii) comprises a nucleic acid encoding a miRNA or shRNA targeting a mRNA encoding a dominant negative mutant thereof;
(iii) comprises a nucleic acid encoding an antibody or antibody binding fragment that specifically binds to a dominant negative mutant thereof; or
(ivii) comprises a nucleic acid encoding a protein that inhibits proliferation of rod cells, optionally wherein the protein is a toxin; a prodrug converting enzyme, e.g. thymidine kinase; or a cell cycle inhibitor, e.g. retinoblastoma protein (pRB), p53, p21CIP1, p27KIP1 and p57KIP2;
(v) comprises a nucleic acid encoding a mRNA encoding a dominant negative mutant of a cell cycle inhibitor; and/or
(vi) comprises a nucleic acid encoding a dominant negative mutant of a cell cycle inhibitor.

7. (canceled)

8. The polynucleotide of claim 1, comprising one or more further nucleotide sequence elements selected from the group consisting of:
(i) a polyadenylation signal (PAS), optionally wherein the PAS comprises or consists of a Simian-Virus 40 (SV40) PAS and/or the PAS comprises or consists of a nucleic acid according to SEQ ID NO: 4 or functional variants thereof;
(ii) one or two inverted terminal repeat (ITR) sequences, optionally wherein:
the ITR sequence is an adeno-associated virus (AAV) ITR,
the ITR sequence is an AAV ITR wherein the AAV is AAV serotype 2, 5, 8, or 9, and/or
the polynucleotide is flanked at the 5′ end with an L-ITR and at the 3′ end with an R-ITR, optionally wherein the L-ITR comprises or consists of the sequence according to SEQ ID NO: 5 or variants thereof and/or the R-ITR comprises or consists of the sequence according to SEQ ID NO: 6 or variants thereof; and
(iii) viral nucleotide sequences necessary to form an infectious viral vector, preferably an adenovirus, a retrovirus, a lentivirus, a vaccinia/poxvirus, or a herpesvirus vector, in particular herpes simplex virus (HSV) vector.

9-14. (canceled)

15. The polynucleotide of claim 1, wherein the total length of the polynucleotide is 5200 bases or less, preferably 5100 bases or less, more preferably 5000 bases of less.

16. A plasmid comprising the polynucleotide of claim 1, optionally wherein the plasmid comprises a nucleic acid sequence according to SEQ ID NOs: 7, 42-44, or variants thereof.

17. (canceled)

18. A viral vector comprising the polynucleotide of claim 1, optionally packaged in a virus selected from the group consisting of AAV2, AAV5, AAV8, AAV9 or variants thereof.

19-20. (canceled)

21. A pharmaceutical composition comprising the polynucleotide according to claim 1, and a pharmaceutically acceptable carrier.

22. A method for treating a disease of the retina, comprising administering the polynucleotide according to claim 1 to a patient in need thereof, optionally wherein the disease of the retina is retinal degeneration.

23-26. (canceled)

27. A polynucleotide comprising from 5′ to 3′:
a) a human rhodopsin promoter comprising the nucleic acid sequence according to SEQ ID NO: 9 or variants thereof; and
b) at least one transgene (TG) operably linked to the promoter of a).

28. The polynucleotide according to claim 27, wherein:
(i) the transgene comprises a nucleic acid encoding a protein that maintains or improves a physiological function of rods;
(ii) the transgene comprises a nucleic acid encoding the human rod cyclic nucleotide-gated channel beta subunit (hCNGB1), ABCA4, AIPL1, BEST1, CACNA1F, CLN3, CLRN1, CNGA1, CEP290, CRB1, CRB2, CRX, GPR98, GUCA1A, GUCA1B, MYO7A, NRL, PDE6A, PDE6B, PRPH2, PROM1, RHO, ROM1, RP1, RP2, RPE65, RPGR, SAG, USH1C, USH1G, USH2A or functional fragments or variants thereof;
(iii) the transgene comprises a nucleic acid encoding a miRNA or shRNA targeting a mRNA encoding a dominant negative mutant thereof;
(iv) the transgene comprises a nucleic acid encoding an antibody or antibody binding fragment that specifically binds to a dominant negative mutant thereof;
(v) the transgene comprises a nucleic acid encoding a protein that inhibits proliferation of rod cells, optionally wherein the protein is a toxin; a prodrug converting enzyme, e.g. thymidine kinase; or a cell cycle inhibitor, e.g. retinoblastoma protein (pRB), p53, p21CIP1, p27KIP1 and p57KIP2;
(vi) the transgene comprises a nucleic acid encoding a mRNA encoding a dominant negative mutant of a cell cycle inhibitor; and/or
(vii) the transgene comprises a nucleic acid encoding a dominant negative mutant of a cell cycle inhibitor.

29. (canceled)

30. The polynucleotide of claim 27, comprising one or more further nucleotide sequence elements selected from the group consisting of:
(i) a polyadenylation signal (PAS), optionally wherein the PAS comprises or consists of a Simian-Virus 40 (SV40) PAS;
(ii) one or two inverted terminal repeat (ITR) sequences, optionally wherein: the ITR sequence is an adeno-associated virus (AAV) ITR, and/or the ITR sequence is an AAV ITR wherein the AAV is AAV serotype 2, 5, 8, or 9; and
(iii) viral nucleotide sequences necessary to form an infectious viral vector, preferably an adenovirus, a retrovirus, a lentivirus, a vaccinia/poxvirus, or a herpesvirus vector, in particular herpes simplex virus (HSV) vector.

31-33. (canceled)

34. A viral vector comprising the polynucleotide of claim 27, optionally packaged in a virus selected from the group consisting of AAV2, AAV5, AAV8, AAV9 or variants thereof.

35. (canceled)

36. A method for treating retinal degeneration or retinitis pigmentosa in a subject in need thereof, comprising administering to the subject a therapeutically effective amount of a polynucleotide according to claim 27, optionally wherein the polynucleotide comprises the nucleic acid sequence set forth in SEQ ID NO: 43.

37-38. (canceled)

39. A method for treating retinal degeneration in a subject in need thereof, wherein the retinal degeneration is characterized by a defect or absence of CNGB1 in the retinal cells of the subject, the method comprising administering to the subject a therapeutically effective amount of a viral vector comprising the nucleic acid sequence set forth in SEQ ID NO: 43, optionally wherein the retinal degeneration is CNGB1-linked retinitis pigmentosa or retinitis pigmentosa type 45 (RP45).

40. (canceled)

41. A method for treating CNGB1-linked retinitis pigmentosa or retinitis pigmentosa type 45 (RP45) in a subject in need thereof, comprising subretinal administration to the subject a therapeutically effective amount of a viral vector comprising the nucleic acid sequence set forth in SEQ ID NO: 43.

42. A polynucleotide comprising from 5′ to 3′:
a) a promoter comprising a human rod photoreceptor-specific promoter element (hRPSPE) comprising the nucleic acid sequence according to SEQ ID NO: 1 or variants thereof and a core promoter (CP); and
b) a transgene encoding the human rod cyclic nucleotide-gated channel beta subunit (hCNGB1) operably linked to the promoter of a),
wherein the variant of SEQ ID NO: 1 comprises one or more nucleic acid substitutions outside nucleotide positions 6 to 13, 32 to 40, 70 to 83, and 87 to 94 of SEQ ID NO: 1.

43. A pharmaceutical composition comprising the polynucleotide of claim 42, and a pharmaceutically acceptable carrier.

44. A pharmaceutical composition comprising a viral vector comprising the nucleic acid sequence set forth in SEQ ID NO: 43, and a pharmaceutically acceptable carrier.
